(19) United States
(12) Patent Application Publication
(10) Pub. No.: US 2002/0037290 A1
(43) Pub. Date: Mar. 28, 2002

(54) Compositions comprising heat shock proteins or alpha(2) macroglobulin, antigenic molecules and saponins, and methods of use thereof

(76) Inventor: Garo H. Armen, Manhasset, NY (US)

Correspondence Address: Pennie & Edmonds LLP, 1155 Avenue of the Americas, New York, NY, 10036-2711, US

(21) Appl. No.: 09/909,778

(22) Filed: Jul. 20, 2001

Related U.S. Application Data
(60) Provisional application No. 60/223,133, filed on Aug. 7, 2000.

(51) Int. Cl. A61K 39/395; A61K 38/17

(52) U.S. Cl. 424/178.1; 514/12; 514/26

(57) ABSTRACT

The present invention relates to pharmaceutical compositions and methods for the prevention and treatment of autoimmune diseases, infectious diseases, neurodegenerative diseases, and primary and metastatic neoplastic diseases. In the practice of the invention, the compositions are employed comprising: (a) a heat shock protein (hsp) or an alpha(2)macroglobulin (α2M); (b) a saponin; and, optionally, (c) an antigenic molecule. The antigenic molecule displays the antigenicity of an antigen of: (a) a cell that elicits an autoimmune response; (b) an agent of an infectious disease; (c) a cancerous cell; or (d) a cell or structure associated with a neurodegenerative or amyloid disease. The hsps that can be used in the practice of the invention include but are not limited to hsp70, hsp90, gp96, calreticulin, hsp 110, grp 170, and PDI, alone or in combination with each other. The antigenic molecule can be covalently or noncovalently bound to the hsp or α2M, free in solution, and/or covalently bound to the saponin. The compositions of the invention can be administered alone or in combination with the administration of antigen presenting cells sensitized with an hsp- or α2M-antigenic molecule complex.

This application claims the benefit under 35 U.S.C. §119(e) of U.S. Provisional Application No. 60/223,133 filed Aug. 7, 2000, which is incorporated by reference herein in its entirety.

### 1. FIELD OF THE INVENTION

The present invention relates to pharmaceutical compositions that are useful for the prevention and treatment of infectious diseases, primary and metastatic neoplastic diseases (i.e., cancer), neurodegenerative or amyloid diseases, and autoimmune diseases, and methods of formulating the compositions. The compositions comprise a heat shock protein (hsp) or alpha(2)macroglobulin (α2M) and a saponin when used for the treatment and prevention of an autoimmune disease. The compositions further comprise an antigenic molecule when used for the treatment or prevention of cancer, infectious disease or neurodegenerative or amyloid disease. Compositions comprising antigenic molecules may also be used for the treatment or prevention of autoimmune disorders.

### 2. BACKGROUND OF THE INVENTION

2.1. Vaccines

Vaccination has eradicated certain diseases such as polio, tetanus, chicken pox, and measles in many countries. This approach has exploited the ability of the immune system to resist and prevent infectious diseases.

Traditional ways of preparing vaccines include the use of inactivated or attenuated pathogens. A suitable inactivation of the pathogenic microorganism renders it harmless as a biological agent but does not destroy its immunogenicity. Injection of these “killed” particles into a host will then elicit an immune response capable of preventing a future infection with a live microorganism. However, a major concern in the use of inactivated pathogens as vaccines is the failure to inactivate all the microorganisms. Even when complete microorganism inactivation is accomplished, the immunity achieved is still often incomplete and/or short-lived, requiring multiple immunizations to sustain an immune response (presumably since killed pathogens do not multiply in their host or for other unknown reasons). Finally, the inactivation process may alter the microorganism's antigens, rendering them less effective as immunogens.

Attenuation refers to the production of strains of pathogenic microorganisms which have essentially lost their disease-producing ability. One way to accomplish this is to subject the microorganism to unusual growth conditions and/or frequent passage in cell culture. Mutants are then selected which have lost virulence but yet are capable of eliciting an immune response. Attenuated pathogens often make good immunogens as they actually replicate in the host cell and elicit long-lasting immunity. However, several problems are encountered with the use of live vaccines, the most worrisome being insufficient attenuation and the risk of reversion to virulence.

An alternative to the above methods is the use of subunit vaccines. Subunit vaccines involve immunization only with a microorganism's components which contain the relevant immunological material. Another promising alternative is the use of DNA or RNA as vaccines. Such genetic vaccines have progressed from idea to reality and are being investigated in clinical trials (see, Weiner and Kennedy, July 1999, Scientific American, pp. 50-57).

Vaccines are often formulated to contain various adjuvants in addition to antigen. Adjuvants aid in attaining a more durable and higher level of immunity using smaller amounts of antigen or fewer doses than if the antigen were administered alone. However, the mechanism(s) of adjuvants is not completely understood and is still unpredictable (see Suzue et al., 1996, Basel: Birkhauser Verlag, 454-55).

2.2. Immune Responses

An organism's immune system responds to pathogens or other harmful agents by two basic mechanisms—a humoral response and a cell-mediated response (see Alberts et al., 1994, Molecular Biology of the Cell, pp. 1195-96). When resting B cells are activated by antigen to proliferate and mature into antibody-secreting cells, they produce and secrete antibodies with a unique antigen-binding site. This antibody-secreting reaction is known as the humoral response. On the other hand, the diverse responses of T cells are collectively called cell-mediated immune reactions. There are two main classes of T cells—cytotoxic T cells and helper T cells. Cytotoxic T cells directly kill cells that are infected with a virus or some other intracellular microorganism. Helper T cells, by contrast, help stimulate the responses of other cells: they help activate macrophages, dendritic cells and B cells, for example (Alberts et al., 1994, Molecular Biology of the Cell, p. 1228). Both cytotoxic T cells and helper T cells recognize antigen in the form of peptide fragments that are generated by the degradation of foreign protein antigens inside the target cell, and both, therefore, depend on major histocompatibility complex (MHC) molecules, which bind these peptide fragments, carry them to the cell surface, and present them there to the T cells (Alberts et al., Id.). MHC molecules are typically found in abundance on antigen-presenting cells (APCs).

Srivastava et al. demonstrated immune response to methylcholanthrene-induced sarcomas of inbred mice (1988, Immunol. Today 9:78-83). In these studies, it was found that the molecules responsible for the individually distinct immunogenicity of these tumors were glycoproteins of 96 kDa (gp96) and intracellular proteins of 84 to 86 kDa (Srivastava et al., 1986, Proc. Natl. Acad. Sci. USA 83:3407-3411; Ullrich et al., 1986, Proc. Natl. Acad. Sci. USA 83:3121-3125). Immunization of mice with gp96 or p84/86 isolated from a particular tumor rendered the mice immune to that particular tumor, but not to antigenically distinct tumors. Isolation and characterization of genes encoding gp96 and p84/86 revealed significant homology between them, and showed that gp96 and p84/86 were, respectively, the endoplasmic reticular and cytosolic counterparts of the same heat shock proteins (Srivastava et al., 1988, Immunogenetics 28:205-207; Srivastava et al., 1991, Curr. Top. Microbiol. Immunol. 167:109-123). Further, hsp70 was shown to elicit immunity to the tumor from which it was isolated but not to antigenically distinct tumors. However, hsp70 depleted of peptides was found to lose its immunogenic activity (Udono and Srivastava, 1993, J. Exp. Med. 178:1391-1396). These observations suggested that the heat shock proteins are not immunogenic per se, but instead form noncovalent complexes with antigenic peptides, and the complexes elicit specific immunity to the antigenic peptides (Srivastava, 1993, Adv. Cancer Res. 62:153-177; Udono et al., 1994, J. Immunol., 152:5398-5403; Suto et al., 1995, Science, 269:1585-1588).

Noncovalent complexes of hsps and peptide, purified from cancer cells, can be used for the treatment and prevention of cancer and have been described in PCT publications WO 96/10411, dated Apr. 11, 1996, and WO 97/10001, dated Mar. 20, 1997 (U.S. Pat. No. 5,750,119 issued Apr. 12, 1998, and U.S. Pat. No. 5,837,251 issued Nov. 17, 1998, respectively, each of which is incorporated by reference herein in its entirety). The isolation and purification of stress protein-peptide complexes has been described, for example, from pathogen-infected cells, and can be used for the treatment and prevention of infection caused by pathogens such as viruses and other intracellular pathogens, including bacteria, protozoa, fungi and parasites (see e.g., PCT Publication WO 95/24923, dated Sep. 21, 1995). Immunogenic stress protein-peptide complexes can also be prepared by in vitro complexing of stress protein and antigenic peptides, and the uses of such complexes for the treatment and prevention of cancer and infectious diseases has been described in PCT publication WO 97/10000, dated Mar. 20, 1997 and U.S. Pat. No. 6,030,618 issued Feb. 29, 2000. The use of stress protein-peptide complexes for sensitizing antigen presenting cells in vitro for use in adoptive immunotherapy is described in PCT publication WO 97/10002, dated Mar. 20, 1997 (see also, U.S. Pat. No. 5,985,270 issued Nov. 16, 1999).

2.3. Heat Shock Proteins and Their Roles in Antigen Presentation

2.3.1. Heat Shock Proteins

Heat shock proteins (hsps), also referred to as stress proteins, were first identified as proteins synthesized by cells in response to heat shock. hsps have been classified into five families based on molecular weight, i.e. hsp100, hsp90, hsp70, hsp60, and smhsp. Many members of these families were found subsequently to be induced in response to other stressful stimuli, including nutrient deprivation, metabolic disruption, oxygen radicals, and infection with intracellular pathogens (see Welch, May 1993, Scientific American 56-64; Young, 1990, Annu. Rev. Immunol. 8:401-420; Craig, 1993, Science 260:1902-1903; Gething et al., 1992, Nature 355:33-45; and Lindquist et al., 1988, Annu. Rev. Genetics 22:631-677).

Heat shock proteins are among the most highly conserved proteins in existence. For example, DnaK, the hsp70 from E. coli, has about 50% amino acid sequence identity with hsp70 proteins from excoriates (Bardwell et al., 1984, Proc. Natl. Acad. Sci. 81:848-852). The hsp60 and hsp90 families also show similarly high levels of intra-family conservation (Hickey et al., 1989, Mol. Cell. Biol. 9:2615-2626; Jindal, 1989, Mol. Cell. Biol. 9:2279-2283). In addition, it has been discovered that the hsp60, hsp70 and hsp90 families are composed of proteins that are related to the stress proteins in sequence, for example, having greater than 35% amino acid identity, but whose expression levels are not altered by stress.

Studies of the cellular response to heat shock and other physiological stresses revealed that the hsps are involved not only in cellular protection against these adverse conditions, but also in essential biochemical and immunological processes in unstressed cells. The hsps accomplish different kinds of chaperoning functions. For example, there has been a suggestion that members of the hsp70 family, located in the cell cytoplasm, nucleus, mitochondria, or endoplasmic reticulum, are involved in the presentation of antigens to the cells of the immune system. Moreover, members of the hsp70 family are known to be involved in the transfer, folding and assembly of proteins in normal cells (Lindquist et al., 1988, Ann. Rev. Genetics 22:631-677). The hsps are capable of binding proteins or peptides, and of releasing the bound proteins or peptides in the presence of adenosine triphosphate (ATP) or low pH.

2.3.2. Antigen Presentation

Major histocompatibility complex (MHC) molecules present antigens on the cell surface of antigen-presenting cells. Cytotoxic T lymphocytes (CTLs) then recognize MHC molecules and their associated peptides and kill the target cell. Antigens are processed by two distinct antigen processing routes depending upon whether their origin is intracellular or extracellular. Intracellular or endogenous protein antigens, i.e., antigens synthesized within the antigen-presenting cell, are presented by MHC class I (MHC I) molecules to CD8+ cytotoxic T lymphocytes. On the other hand, extracellular or exogenously synthesized antigenic determinants are presented on the cell surface of “specialized” or “professional” APCs (macrophages, for example) by MHC class II molecules to CD4+ T cells (see generally, Fundamental Immunology, W. E. Paul (ed.), New York: Raven Press, 1984). This compartmental segregation of antigen processing routes is important to prevent tissue destruction that could otherwise occur during an immune response as a result of shedding of neighboring cell MHC I antigens.

The heat shock protein gp96 chaperones a wide array of peptides, depending upon the source from which gp96 is isolated (for review, see Srivastava et al., 1998, Immunity 8: 657-665). Tumor-derived gp96 carries tumor-antigenic peptides (Ishii et al., 1999, J. Immunology 162:1303-1309), gp96 preparations from virus-infected cells carry viral epitopes (Suto and Srivastava, 1995, Science 269:1585-1588; Nieland et al., 1998, Proc. Natl. Acad. Sci. USA 95:1800-1805), and gp96 preparations from cells transfected with model antigens such as ovalbumin or β-galactosidase are associated with the corresponding epitopes (Arnold et al., 1995, J. Exp. Med.182:885-889; Breloer et al., 1998, Eur. J. Immunol. 28:1016-1021). The association of gp96 with peptides occurs in vivo (Menoret and Srivastava, 1999, Biochem. Biophys. Research Commun. 262:813-818). Gp96-peptide complexes, whether isolated from cells (Tamura et al., 1997, Science 278:117-120), or reconstituted in vitro (Blachere et al., 1997, J. Exp. Med. 186:1183-1406) are excellent immunogens and have been used extensively to elicit CD8+ T cell responses specific for the gp96-chaperoned antigenic peptides.

The capacity of gp96-peptide complexes to elicit an immune response is dependent upon the transfer of the peptide to MHC class I molecules of antigen-presenting cells (Suto and Srivastava, 1995, supra). Endogenously synthesized antigens chaperoned by gp96 in the endoplasmic reticulum (ER) can prime antigen-specific CD8+ T cells (or MHC I-restricted CTLs) in vivo; this priming of CD8+ T cells requires macrophages. However, the process whereby exogenously-introduced gp96-peptide complexes elicit the antigen-specific CD8+ T cell response is not completely understood since there is no established pathway for the translocation of extracellular antigens into the class I presentation machinery. Yet antigenic peptides of extracellular origin associated with hsps are somehow salvaged by macrophages, channeled into the endogenous pathway, and presented by MHC I molecules to be recognized by CD8+ lymphocytes (Suto and Srivastava, 1995, supra; Blachere et al., 1997, J. Exp. Med. 186:1315-22).

Several models have been proposed to explain the delivery of extracellular peptides for antigen presentation. One proposal, known as the “direct transfer” model, suggests that hsp-chaperoned peptides are transferred to MHC I molecules on the cell surface of macrophages for presentation to CD8+ T lymphocytes. Another suggestion is that soluble extracellular proteins can be trafficked to the cytosol via constitutive macropinocytosis in bone marrow-derived macrophages and dendritic cells (Norbury et al., 1997, Eur. J. Immunol. 27:280-288). Yet another proposed mechanism is that hsps are taken up by the MHC class I molecules of the macrophage, which stimulate the appropriate T cells (Srivastava et al., 1994, Immunogenetics 39:93-98). Others have suggested that a novel intracellular trafficking pathway may be involved for the transport of peptides from the extracellular medium into the lumen of the ER (Day et al., 1997, Proc. Natl. Acad. Sci. 94:8064-8069; Nicchitta, 1998, Curr. Opin. in Immunol. 10:103-109). Further suggestions include the involvement of phagocytes which: (a) possess an ill-defined pathway to shunt protein from the phagosome into the cytosol where it would enter the normal class I pathway; and/or (b) digest ingested material in lysosomes and regurgitate peptides for loading on the surface to class I molecules (Bevan, 1995, J. Exp. Med. 182:639-41).

Still others have proposed a receptor-mediated pathway for the delivery of extracellular peptides to the cell surface of APCs for antigen presentation. In view of the extremely small quantity of gp96-chaperoned antigenic peptides required for immunization (Blachere et al., 1997, supra), and the strict dependence of immunogenicity of gp96-peptide complexes on functional antigen presenting cells (APCs) (Udono et al., 1994, Proc. Natl. Acad. Sci. U.S.A. 91:3077-3081), APCs had been proposed to possess receptors for gp96 (Srivastava et al., 1994, Immunogenetics 39:93-98). The mannose receptor is thought to be used in the uptake of gp96 (Ciupitu et al., 1998, J. Exp. Med., 187:685-691). The alpha(2)macroglobulin receptor, also known as CD91, has proven to be a more universal receptor for hsps, with binding to gp96, hsp90, hsp70, and calreticulin having already been demonstrated, as further discussed in Section 2.5 below.

Antigen-presenting cells (APCs), such as macrophages and dendritic cells, are key components of innate and adaptive immune responses. Antigens are generally ‘presented’ to T cells or B cells on the surfaces of other cells, the APCs. APCs can trap lymph- and blood-borne antigens and, after internalization and degradation, present antigenic peptide fragments, bound to cell-surface molecules of the major histocompatibility complex (MHC), to T cells. APCs may then activate T cells (cell-mediated response) to clonal expansion, and these daughter cells may either develop into cytotoxic T cells or helper T cells, which in turn activate B (humoral response) cells with the same MHC-bound antigen to clonal expansion and specific antibody production (see Alberts et al., 1994, Molecular Biology of the Cell, pp. 1238-45).

Stimulation of T cells involves a number of accessory molecules expressed by both T cells and APCs. Co-stimulatory molecules are those accessory molecules that promote the growth and activation of the T cell, e.g., B7-1, B7-2, CD40, ICAM-1 and MHC II on the APC surface, and CD28, CD40L, T-cell antigen surface receptors (TCRs) and CD4 on the T cell surface (see e.g., Banchereau and Steinman, 1998, Nature 392:245-252). Upon stimulation, co-stimulatory molecules induce release of cytokines, such as interleukin 1 (IL-1) or interleukin 2 (IL-2), interferon, etc., which promote T cell growth and expression of surface receptors (See e.g., Paul, 1989, Fundamental Immunology, pp. 109-10).

2.4. Adoptive Immunotherapy

Adoptive immunotherapy of cancer refers to a therapeutic approach in which immune cells with an antitumor reactivity are administered to a tumor-bearing host, with the aim that the cells mediate, either directly or indirectly, the regression of an established tumor. Transfusion of lymphocytes, particularly T lymphocytes, falls into this category and investigators at the National Cancer Institute (NCI) have used autologous reinfusion of peripheral blood lymphocytes or tumor-infiltrating lymphocytes (TIL) T cell cultures from biopsies of subcutaneous lymph nodules, to treat several human cancers (Rosenberg, S. A., U.S. Pat. No. 4,690,914, issued Sep. 1, 1987; Rosenberg, S. A. et al., 1988, N. England J. Med. 319:1676-1680). For example, TIL expanded in vitro in the presence of interleukin (IL)-2 have been adoptively transferred to cancer patients, resulting in tumor regression in select patients with metastatic melanoma. Melanoma TIL grown in IL-2 have been identified as activated T-lymphocytes CD3+ HLA-DR+, which are predominantly CD8+ cells with unique in vitro antitumor properties. Many long-term melanoma TIL cultures lyse autologous tumors in a specific MHC class I- and T cell antigen receptor dependent manner (Topalian et al., 1989, J. Immunol. 142:3714). However, studies of TIL derived from other types of tumors have revealed only scant evidence for cytolytic or proliferative antitumor immune specificity (Topalian et al., 1990, in Important Advances in Oncology, V. T. DeVita, S. A. Hellman and S. A. Rosenberg, eds. J. B. Lippincott, Philadelphia, pp. 19-41). In addition, the toxicity of the high-dose IL-2+ activated lymphocyte treatment advocated by the NCI group has been considerable, including high fevers, severe rigors, hypotension, damage to the endothelial wall due to capillary leak syndrome, and various adverse cardiac events such as arrhythmias and myocardial infarction (Rosenberg et al., 1988, N. England J. Med. 319:1676-1680).

2.5. α2-Macroglobulin

The α-macroglobulins are members of a protein superfamily of structurally related proteins which also comprises complement components C3, C4 and C5. The human plasma protein alpha(2)macroglobulin (α2M) is a 720 kDa homotetrameric protein primarily known as proteinase inhibitor and plasma and inflammatory fluid proteinase scavenger molecule (for review see Chu and Pizzo, 1994, Lab. Invest. 71:792). Alpha (2) macroglobulin is synthesized as a 1474 amino acid precursor, the first 23 of which function as a signal sequence that is cleaved to yield a 1451 amino acid mature protein (Kan et al., 1985, Proc. Natl. Acad. Sci. U.S.A. 82:2282-2286).

Alpha(2)macroglobulin promiscuously binds to proteins and peptides with nucleophilic amino acid side chains in a covalent manner (Chu et al., 1994, Ann. N.Y. Acad. Sci. 737:291-307) and targets them to cells which express the α2M receptor (α2MR) (Chu and Pizzo, 1993, J. Immunol. 150:48). Binding of α2M to the α2MR is mediated by the C-terminal portion of α2M (Holtet et al., 1994, FEBS Lett. 344:242-246) and key residues have been identified (Nielsen et al., 1996, J. Biol. Chem. 271:12909-12912).

Generally known for inhibiting protease activity, α2M binds to a variety of proteases thorough multiple binding sites (see, e.g., Hall et al., 1981, Biochem. Biophys. Res. Commun. 100(1):8-16). Protease interaction with α2M results in a complex structural rearrangement called transformation, which is the result of a cleavage within the “bait” region of α2M after the proteinase becomes “trapped” by thioesters. The conformational change exposes residues required for receptor binding, allowing the α2M-proteinase complex to bind to the α2MR. Methylamine can induce similar conformational changes and cleavage as that induced by proteinases. The uncleaved form of α2M, which is not recognized by the receptor, is often referred to as the “slow” form (s-α2M). The cleaved form is referred to as the “fast” form (f-α2M) (reviewed by Chu et al., 1994, Ann. N.Y. Acad. Sci. 737:291-307).

Studies have shown that, in addition to its proteinase-inhibitory functions, α2M, when complexed to antigens, can enhance the antigens' ability to be taken up by antigen presenting cells such as macrophages and presented to T cell hybridomas in vitro by up to two orders of magnitude (Chu and Pizzo, 1994, Lab. Invest. 71:792), and induce T cell proliferation (Osada et al., 1987, Biochem. Biophys. Res. Commun.146:26-31). Further evidence suggests that complexing antigen with α2M enhances antibody production by crude spleen cells in vitro (Osada et al., 1988, Biochem. Biophys. Res. Commun. 150:883) and elicits an in vivo antibody responses in experimental rabbits (Chu et al., 1994, J. Immunol. 152:1538-1545) and mice (Mitsuda et al., 1993, Biochem. Biophys. Res. Commun. 101:1326-1331). However, none of these studies have shown whether α2M-antigen complexes are capable of eliciting cytotoxic T cell responses in vivo.

α2M can form complexes with antigens, which are taken up by antigen presenting cells (“APCs”) via the α2MR, also known as LDL (low-density lipoprotein) Receptor-Related Protein (“LRP”) or CD91 (see provisional patent application No. 60/209,266 filed Jun. 2, 2000, which is incorporated by reference herein in its entirety). α2M directly competes for the binding of heat shock protein gp96 to the α2MR, indicating that α2M and hsps may bind to a common recognition site on the α2MR (Binder et al., 2000, Nature Immunology 1(2), 151-154). Additionally, α2M-antigenic peptide complexes prepared in vitro can be administered to animals to generate a cytotoxic T cell response specific to the antigenic molecules (Binder et al., 2001, J. Immunol. 166:4968-72). Thus, because hsps and α2M have a number of common functional attributes, such as the ability to bind peptide, the recognition and uptake by the α2MR, and the stimulation of a cytotoxic T cell response, α2M can be used for immunotherapy against cancer, infectious disease and neurodegenerative or disease.

2.6. Saponins

Quillaja saponins are a mixture of triterpene glycosides extracted from the bark of the tree Quillaja saponaria. They have long been recognized as immune stimulators that can be used as vaccine adjuvants, (Campbell and Peerbaye, 1992, Res. Immunol. 143(5):526-530), and a number of commercially available complex saponin extracts have been utilized as adjuvants. Crude saponins have been extensively employed as adjuvants in veterinary vaccines against foot and mouth disease, and in amplifying the protective immunity conferred by experimental vaccines against protozoal parasites such as Trypanosoma cruzi plasmodium and also the humoral response to sheep red blood cells (SRBC) (Bomford, 1982, Int. Arch. Allerg. Appl. Immun. 67:127).

The first commercially available Quillaja saponin adjuvants were crude extracts which, because of their variability, were not desirable for use in veterinary practice or in pharmaceutical compositions for man. An early attempt to purify Quillaja saponin adjuvants was made by Dalsgaard (1974, Archiv fuer die gesamte Virusforschung 44:243). Dalsgaard partially purified an aqueous extract of the saponin adjuvant material from Quillaja saponaria Molina. However, while Dalsgaard's preparation, “Quil-A,” was a definite improvement over the previously available commercial saponins, it still exhibited considerable heterogeneity.

Subsequent analysis via high-pressure liquid chromatography showed that Quil A was in fact a heterogeneous mixture of structurally related triterpene glycosides (U.S. Pat. No. 5,057,540; Kersten et al., 1988, Infect. Immun. 56:432-438; Kensil et al., 1991, J. Immunol. 146:431-437; Kensil et al., 1991, J. Am. Vet. Med. Assoc. 199:1423-1427). However, not all of these saponins were active as adjuvants.

The four most predominant purified Quillaja saponins are QS-7, QS-17, QS-18, and QS-21 (alternatively identified as QA-7, QA-17, QA-18, and QA-21). These saponins have been purified by HPLC and low pressure silica chromatography and were found to be adjuvant active, although differing in biological activities such as hemolysis and toxicity in mice. In particular, QS-21 and QS-7 were found to be least toxic in mice (Kensil et al., 1991, J. Immunol. 146:431-437).

Due to its potent adjuvant activity and low toxicity, QS-21 (commercially available as the “Stimulon®” adjuvant) has been identified as a useful immunological adjuvant (Kensil et al., 1995, “Structural and Immunological Characterization of the Vaccine Adjuvant QS-21,” in Vaccine Design: The Subunit and Adjuvant Approach, Powell and Newman eds., Plenum Press, New York). QS-21 is a complex triterpene glycoside of quillaic acid. QS-21 is glycosylated at triterpene carbon 3, triterpene carbon 28, and carbon 5 of the second fatty acyl unit in a fatty acid domain.

More recently, QS-21 was further purified using hydrophilic interaction chromatography (HILIC) and resolved into two peaks, QS-21-V1 and QS-21-V2, which have been shown to be chemically different compounds. In C57BL/6 mice immunized with vaccines consisting of ovalbumin and either QS-21, QS-1-V1, or QS-21-V2, both of the individual components QS-21-V1 and QS-21-V2 are comparable in adjuvant effect to the original QS-21 peak (containing a mixture of 3:2 QS-21-V1 and QS-21-V2) for boosting the IgG subclasses IgG1, IgG2b, and IgG2 as well as the total IgG titer (U.S. Pat. No. 5,583,112, the entire contents of which are hereby incorporated by reference).

Quillaja saponins are structurally distinct from the saponins derived from other plant species. Two structural features that distinguish Quillaja saponaria saponins from those of other plant species are a fatty acid domain and a triterpene aldehyde at carbon 4 of the triterpene. (Kensil et al., 1995, “Structural and Immunological Characterization of the Vaccine Adjuvant QS-21,” in Vaccine Design: The Subunit and Adjuvant Approach, Powell and Newman eds., Plenum Press, New York). Modifications to the aldehyde on the triterpene indicate that this functional group may be involved in the adjuvant mechanism (Soltysik et al., 1995, Vaccine 13(15):1403-1410).

Quillaja saponins, particularly QS-7, QS-17, QS-18, and QS-21, have been found to be excellent stimulators of antibody response to soluble T-dependent protein antigens, “subunit antigens”, which are poorly immunogenic and require a potent adjuvant for maximization of immune responses. Examples of purified subunit antigens for which saponin adjuvants that augment the IgG response in mice include keyhole limpet hemocyanin (KLH), HIV-1 gp120 (Bomford et al., 1992, AIDS Res. Hum. Retroviruses 8:1765), and influenza nucleoprotein (Brett et al., 1993, Immunology 80:306). QS-7, QS-17, QS-18 and QS-21 have also been shown to stimulate potent antibody responses in mice to the antigens bovine serum albumin and cytochrome b₅ (Kensil et al., 1991, J. Immunol. 146:431). The level of antibody response induced by these purified saponins was comparable to other commonly used adjuvants, e.g., complete Freund's adjuvant, and superior to aluminum hydroxide.

QS-21 has also been shown to enhance antibody responses to T-independent antigens, including unconjugated bacterial polysaccharides (White et al., 1991, “A purified saponin acts as an adjuvant for a T-independent antigen,” in: Immunobiology of Proteins and Peptides, Vol. VI (Atassi ed.), Plenum Press, New York, pp. 207-210). The immunogenicity of the vaccine was further increased by conjugating diphtheria toxoid to the polysaccharide. QS21 enhanced the antibody response to the polysaccharide as well as the carrier, including IgG2a, IgG2b, and IgG3 responses (Coughlin et al.,1995, Vaccine 13(1):17-21).

The ability of adjuvants to modulate the isotype distribution and IgG subclass distribution of antibody response to an antigen through the promotion of Ig subclass switching has important implications for immunity to many bacterial and viral vaccines. QS-7, QS-17, QS-18, and QS-21 stimulate IgG2a response to cytochrome b5 after administration with saponin doses of 20 μg (Kensil et al., 1991, J. Immunol 146:431). In this regard, QS-21 shifts predominant IgG1 responses to a profile that includes significant IgG2b and IgG2a responses. For example, QS-21 has been shown to stimulate antigen-specific IgG2a to a number of antigens, including Borrelia burgdorferi outer surface proteins OspA and OspB (Ma et al., 1994, Vaccine 12(10):925), feline leukemia virus (FeLV) envelope gp70 (Kensil et al., 1991, J. Am. Vet. Med. Assoc.10:1423), human cytomegalovirus (HCMV) envelope protein gB (Britt et al., 1995, J. Infect. Dis. 171:18), respiratory synctial virus (RSV) purified fusion protein (Hancock et al., 1995, Vaccine 13(4):391), and tetanus toxoid (Coughlin et al., 1995, Vaccine 13(1):17). QS-21 has also been shown to induce boostable antibody responses (Britt et al., 1995, J. Infect. Dis. 171:18-25; Helling et al., 1995, Cancer Res. 55:2783-2788).

The ability of the QS-21 adjuvant to induce class I major histocompatibility complex (MHC) antigen-restricted cytotoxic T-lymphocyte responses (CTL) after immunization with soluble proteins is a characteristic of saponin adjuvants. A number of studies have shown the ability of QS-21 to induce potent cytotoxic T-lymphocyte (CTL) responses to various antigens, including ovalbumin (Wu et al., 1994, Cell. Immunol. 154:394-406; Newman et al., 1992, J. Immunol. 148(8):2357-2362), recombinant HIV-1 gp160 protein (Wu et al., 1992, J. Immunol. 148:1519), respiratory syncytial virus (“RSV”) purified fusion protein (Hancock et al., 1995, Vaccine 13(4):391), and subunit SIV_{mac251} gag and env (Newman et al., 1994, AIDS Res. Hum. Retroviruses 10(7):853).

Most of the saponin adjuvant studies have been carried out in mice. However, the adjuvant activity of saponins is not limited to mice; it has also been demonstrated in guinea pigs, rabbits, pigs, sheep, cattle, and nonhuman primates. An adjuvant effect from QS-21 has been observed in cats, guinea pigs, dogs, nonhuman primates, and humans (Kensil et al., 1995, “Structural and Immunological Characterization of the Vaccine Adjuvant QS-21,” in Vaccine Design: The Subunit and Adjuvant Approach, Powell, M. F. and Newman, M. J. eds., Plenuim Press, New York).

Phase 1 human trials of QS-21 with GM2 ganglioside-keyhole limpet haemocyanin conjugate vaccine have been conducted in patients with malignant melanoma (Livingston et al., 1994, Vaccine 12:1275-1280. Increased immunogenicity after administration with QS-21 adjuvant was observed (Helling et al., 1995, Cancer Res. 55:2783-2788). In another set of clinical trials, QS-21 was found to be a potent immunological adjuvant that significantly increased the serological response of melanoma patients to the murine antiidiotype antibody MELIMMUNE-1 (Livingston et al., 1995, Vaccine Res. 4(2):87).

The immune adjuvant effect of saponins is dependent upon dose. Depending upon the antigen and the species, a minimum dose level of QS-21 is required for optimum response (Kensil et al., 1991, J. Immunol.; Kensil et al., 1993, Vaccine Res.; Newman et al., 1992, J. Immunol.; Livingston et al., 1994, Vaccine. Below this minimum dose, the immune adjuvant effect is suboptimal (either low level or absent). QS-7 also has a dose response curve (Kensil et al., 1991, J. Immunol.).

### 3. SUMMARY OF THE INVENTION

The present invention provides pharmaceutical compositions comprising an hsp or α2M and a saponin, which compositions produce enhanced inhibition of autoimmune responses when administered in lower doses relative to compositions comprising an hsp or α2M in the absence of a saponin. The compositions are therefore useful for treating autoimmune diseases. Accordingly, the present invention provides pharmaceutical compositions comprising a purified hsp and a saponin or a purified α2M and a saponin. The pharmaceutical compositions optionally further comprise an antigenic molecule. The pharmaceutical compositions can be used to treat or prevent an autoimmune disease in an individual when administered to the individual in an amount effective to treat or prevent the autoimmune disease.

The present invention further provides compositions comprising an hsp or α2M, an antigenic molecule, and a saponin, which have enhanced antigenicity or immunogenicity relative to compositions comprising hsps or α2M and antigenic molecules in the absence of a saponin. Such compositions are useful for treating or preventing cancer or an infectious disease or a neurodegenerative or amyloid disease. Accordingly, the present invention provides pharmaceutical compositions comprising a purified hsp or α2M, a first antigenic molecule, and a saponin.

In one embodiment, the first antigenic molecule is not bound to the saponin or to the hsp or α2M. In another embodiment, the pharmaceutical compositions further comprise a second antigenic molecule. The second antigenic molecule can be covalently or non-covalently bound to the hsp or α2M. Optionally, the pharmaceutical compositions further comprise a third antigenic molecule, which can be covalently bound to the saponin.

In other embodiments, the first antigenic molecule is covalently or non-covalently bound to the hsp or α2M. Optionally, the saponin is covalently bound to a second antigenic molecule.

The hsp present in a pharmaceutical composition of the invention can be hsp70, hsp90, gp96, calreticulin, hsp 110, grp170, PDI, or a mixture of two or more of the foregoing. In certain specific embodiment, the hsp or α2M is recombinantly produced as a fusion protein with the second antigenic molecule. In a preferred embodiment, the saponin is QS-7, QS-21, QS-21-V1, or QS-21-V2.

The compositions of the invention comprising hsps or α2M, antigenic molecules and saponins can be used to elicit an immune response against cancer or an agent of infectious disease, by administering to an individual an amount effective to elicit an immune response in the individual. Where eliciting an immune response against a type of cancer is desired, an antigenic molecule is used which displays antigenicity of an antigen of the type of cancer. Where eliciting an immune response against an agent of an infectious disease is desired, an antigenic molecule is used which displays antigenicity of an antigen of the agent of infectious disease. In other embodiments, the compositions of the invention that comprise an antigenic molecule that displays antigenicity of an antigen of a type of cancer are used to treat or prevent the type of cancer, the compositions of the invention that comprise an antigenic molecule that displays antigenicity of an antigen of an agent of an infectious disease are used to treat or prevent said infectious disease; and the compositions of the invention that comprise an antigenic molecule associated with a neurodegenerative disease or an amyloid disease are used to treat or prevent said neurodegenerative or amyloid disease.

Prophylactic and therapeutic dosage regimens and kits are also provided by the invention.

### 4. DETAILED DESCRIPTION OF THE INVENTION

Methods and pharmaceutical compositions for the prevention and treatment of primary and metastatic neoplastic diseases (also herein referred to as “cancers”), autoimmune diseases, infectious diseases and neurodegenerative and amyloid disease and for eliciting an immune response in an individual, are described. Particular compositions of the invention and their properties and methods of use are described in the sections and subsections which follow.

The invention is based, in part, on the identification of saponins as useful adjuvants for enhancing the immune response to administration of low doses of hsps or α2M alone, or to administration of lower doses of hsps or α2M complexed covalently or non-covalently to antigenic molecules. The use of saponins, and in particular QS-7, QS-17, QS-18, QS-21, QS-21-V1, and/or QS-21-V2, permits the use of reduced amounts of hsps or α2M and is particularly useful when the quantities of hsps or α2M or antigen would otherwise be limiting. The saponins can be used alone or covalently complexed to antigenic molecules.

The compositions of the invention minimally comprise one or more hsps or α2M and a saponin adjuvant. Such compositions, which substantially lack antigenic molecules, are particularly useful in treating an autoimmune disorder. “Antigenic molecule” as used herein refers to a peptide or other molecule with which hsps are endogenously associated in vivo (e.g., in precancerous or cancerous tissue), as well as exogenous antigens/immunogens (i.e., with which the hsps are not complexed in vivo) or antigenic/immunogenic fragments and derivatives thereof. Such exogenous antigens and fragments and derivatives (both peptide and non-peptide) thereof for use in complexing with hsps or α2M, can be selected from among those known in the art, as well as those readily identified by standard immunoassays known in the art by detecting the ability to bind antibody or MHC molecules (antigenicity) or generate immune response (immunogenicity). Compositions comprising an hsp or α2M and a saponin adjuvant which further comprise an antigenic molecule are useful in eliciting an immune response against the antigenic molecule or a cell in which the antigenic molecule is present, such as a cancer cell, a cell infected with an infectious organism or a cell or structure, e.g., extracellular deposits or plaques comprising peptide and/or protein fibrils, that displays the hallmarks of a neurodegenerative or amyloid disease. In certain embodiments, the outcome of eliciting an immune response is prophylaxis or therapy.

The hsps or α2M, and/or antigenic molecules are preferably autologous to the individual, although they may also be allogeneic. The hsps or α2M and antigenic molecules can be isolated as naturally-occurring complexes, e.g. from cancer cells or cells infected with an infectious agent. Alternatively, the hsps or α2M and/or antigenic molecules can be chemically synthesized or recombinantly produced.

Thus, the compositions can be utilized for the prevention of a variety of cancers, e.g., in individuals who are predisposed as a result of familial history or in individuals with an enhanced risk to cancer due to environmental factors, for the prevention of infectious diseases, e.g., in individuals with enhanced risks of exposure to agents of infectious disease, and for the prevention of neurodegenerative or amyloid diseases, for example in individuals with genetic predispositions to neurodegenerative or amyloid diseases.

In certain embodiment, the compositions of the invention are administered in conjunction with administering one or more biological response modifiers, e.g., IFN-α, IFN-γ, IL-2, IL-4, IL-6, TNF, or other cytokine growth factors affecting the immune cells.

Optionally, adoptive immunotherapy can be used in conjunction with administration of the hsps or α2M, antigenic molecule, and saponin compositions of the invention in treating or preventing cancer, an infectious disease or neurodegenerative or amyloid disease.

The methods of the invention further comprise methods of making the claimed compositions. The saponin, the antigenic molecule and the hsp or α2M are combined under conditions that produce a pharmaceutically effective composition. The saponin can be combined with the antigenic molecule and then antigen-free hsp or α2M is added. Alternatively, the saponin is covalently linked with the antigenic molecule and then combined with antigen-free hsp or α2M. In another embodiment, the antigen-free hsp or α2M is combined with the antigenic molecule and then the saponin added. In another embodiment, antigen-free hsp or α2M, the saponin, and the antigenic molecule are combined simultaneously. In another embodiment, purified hsp or α2M is stripped of bound peptide and antigenic molecule, or antigenic molecule previously covalently linked to saponin, is bound to said hsp or α2M in vitro. In yet another embodiment, aqueous solutions of the saponin, the antigenic molecule and antigen-free hsp or α2M are combined to produce a composition under conditions that permit antigenic molecule binding to the antigen-free hsp or α2M. In yet another embodiment, the saponin is mixed with hsp- or α2M-antigenic molecule complexes that were formed endogenously in cells and purified from the cells. In yet another embodiment, the saponin is mixed with hsp- or α2M-antigenic molecule complexes that are produced in vitro using purified hsp and antigenic molecule. In certain specific embodiments, the hsp or α2M is recombinantly expressed as a fusion protein with an antigenic molecule. In the above methods, multiple hsps (e.g. hsp70, hsp90 and gp96), α2M, antigenic molecules, and saponins may be combined in a single composition of the invention.

4.1. Compositions of the Invention

The hsp or α2M/antigenic molecule/saponin compositions of the invention are administered to elicit an effective specific immune response to the antigenic molecules (and not to the hsp or α2M). In accordance with the methods described herein, hsp or α2M-antigenic molecule complexes are preferably purified in the range of 60 to 100 percent of the total mg protein, or at least 70%, 80% or 90% of the total mg protein. In another embodiment, hsp or α2M -antigenic molecule complexes are purified to apparent homogeneity, as assayed by sodium dodecyl sulfate-polyacrylamide gel electrophoresis.

In preferred embodiments, non-covalent complexes of hsp70, hsp90, gp96 and/or α2M with antigenic molecules are prepared and purified postoperatively from tumor cells obtained from a cancer patient.

In accordance with the methods described herein, immunogenic or antigenic peptides that are endogenously complexed to hsps or α2M can be used as specific antigenic molecules. For example, such peptides may be prepared that stimulate cytotoxic T cell responses against different tumor antigens (e.g., tyrosinase, gp100, melan-A, gp75, mucins, etc.); viral proteins including, but not limited to, proteins of immunodeficiency virus type I (HIV-I), human immunodeficiency virus type II (HIV-II), hepatitis type A, hepatitis type B, hepatitis type C, influenza, Varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus and polio virus; antigenic molecules of neurodegenerative or amyloid diseases, such as β-amyloid or a fragment thereof, an oligomeric Aβ complex or a fragment thereof, an ApoE4-Aβ complex or a fragment thereof, tau protein or a fragment thereof, a mutant amyloid precurser protein or a fragment thereof, a mutant of presenillin or a fragment thereof, α-synuclein or a fragment thereof, or a prion protein, and their antigenic derivatives. In the embodiment wherein the antigenic molecules are peptides noncovalently complexed to hsps or α2M in vivo, the complexes can be isolated from cells, or alternatively, produced in vitro from purified preparations each of hsps or α2M and antigenic molecules.

In another specific embodiment, specific antigens of cancers (e.g., tumors) or infectious agents (e.g., viral antigens, bacterial antigens, etc.) can be obtained by purification from natural sources, by chemical synthesis, or recombinantly, and, through in vitro procedures such as that described below, noncovalently complexed to hsps or α2M.

In another embodiment, the hsp or α2M-specific antigen complexes or antigens obtained by purification from natural sources, by chemical synthesis, or recombinantly, and, through in vitro procedures, are related to an autoimmune disease. These autoimmune diseases include, but are not limited to, insulin-dependent diabetes mellitus (i.e., IDDM, or autoimmune diabetes), multiple sclerosis, systemic lupus erythematosus, Sjogren's syndrome, scleroderma, polymyositis, chronic active hepatitis, mixed connective tissue disease, primary biliary cirrhosis, pernicious anemia, autoimmune thyroiditis, idiopathic Addison's disease, vitiligo, gluten-sensitive enteropathy, Graves' disease, myasthenia gravis, autoimmune neutropenia, idiopathic thrombocytopenia purpura, rheumatoid arthritis, cirrhosis, pemphigus vulgaris, autoimmune infertility, Goodpasture's disease, bullous pemphigoid, discoid lupus, ulcerative colitis, and dense deposit disease. The diseases set forth above, as referred to herein, include those exhibited by animal models for such diseases, such as, for example non-obese diabetic (NOD) mice for IDDM and experimental autoimmune encephalomyelitis (EAE) mice for multiple sclerosis.

In an embodiment wherein an α2M or hsp-specific antigenic molecule complex to be used is a complex that is produced in vivo in cells, exemplary purification procedures such as described in Sections 4.2.1 and 4.2.2 below can be employed. Alternatively, in an embodiment wherein one wishes to use specific antigenic molecules by complexing to hsps in vitro, hsps can be purified for such use from the endogenous hsp-peptide complexes in the presence of ATP or low pH (or chemically synthesized or recombinantly produced). The protocols described herein may be used to isolate hsp-peptide complexes, or the hsps alone, from any eukaryotic cells for example, tissues, isolated cells, or immortalized eukaryote cell lines infected with a preselected intracellular pathogen, tumor cells or tumor cell lines.

In another embodiment, part or all of the first antigen is covalently bound to an hsp to form an hsp-first antigen complex by any means known in the art. In a preferred embodiment, the amount of hsp-first antigen complex is about 0.1 μg or greater. In an alternative first embodiment, α2M is used in place of hsp. In a preferred alternative embodiment, the hsp or α2M and specific antigen are expressed as a recombinant fusion protein. To produce such a recombinant fusion protein, an expression vector is constructed using nucleic acid sequences encoding α2M fused to sequences encoding an antigenic molecule, using recombinant methods known in the art (see Suzue et al., 1997, Proc. Natl. Acad. Sci. U.S.A. 94: 13146-51). α2M-antigenic peptide fusions are then expressed and isolated. By specifically designing the antigenic peptide portion of the molecule, such fusion proteins can be used to elicit an immune response and in immunotherapy against target cancer and infectious diseases.

In another embodiment, part or all of the first antigen is noncovalently bound to an hsp to form an hsp-first antigen complex. In a preferred embodiment, the amount of hsp-first antigen complex is about 0.1 μg or greater. In an alternative second embodiment, α2M is used in place of hsp.

In another embodiment, the part or all of the first antigen is covalently bound to a saponin.

In another embodiment, the pharmaceutical compositions of the invention for the prevention or treatment of an autoimmune disorder comprise an hsp or α2M and may further comprise an antigen that may not elicit a specific immune response to an autoimmune disease.

In another embodiment, the pharmaceutical compositions of the previous embodiments may include unbound antigen (i.e. antigen not complexed with an hsp or α2M).

In one embodiment, the amount of saponin in a pharmaceutical composition of the invention is about 1 micrograms or more. In a preferred embodiment, the amount of saponin in the pharmaceutical composition is from about 10 to about 1000 micrograms. In a particularly preferred embodiment, the amount of saponin is about 100 micrograms.

In other embodiments, the saponin is QS-7, QS-21, QS-21-V1, or QS-21-V2. In a preferred embodiment, the saponin is QS-21. The amount of QS-21 in the pharmaceutical compositions is preferably about 1 microgram or more. In a particularly preferred embodiment, the amount of QS-21 is from about 10 to about 1000 micrograms. In a particularly preferred embodiment, the amount of QS-21 is about 100 micrograms.

In another embodiment, the pharmaceutical composition comprises an hsp-first antigen complex wherein the first antigen is either covalently or noncovalently bound to the hsp, an hsp-second antigen complex wherein the second antigen is either covalently or noncovalently bound to the hsp, and a saponin selected from the group consisting of QS-7, QS-21, QS-21-V1, and QS-21-V2, wherein the amount of specific hsp-first antigen complex is about 0.1 μg or greater and the amount of QS-21 is about 1 microgram or greater.

In another embodiment, the pharmaceutical composition comprises an α2M-first antigen complex wherein the first antigen is either covalently or noncovalently bound to the α2M and QS-21, wherein the amount of QS-21 is about 1 microgram or greater.

In a particularly preferred form of the above hsp-containing embodiments, the hsp is selected from the group consisting of hsp70, hsp90, gp96, calreticulin, hsp 110, grp170, PDI, or a mixture of two or more of the foregoing hsps.

In another particularly preferred form of the above embodiments, an hsp or α2M is purified from cancerous or infected tissues.

In another particularly preferred form of the above embodiments, an hsp or α2M is purified from a cell line. In a specific embodiment, the cell line is transfected with a nucleic acid encoding the hsp or α2M. In other specific embodiments, the cell line is transfected with a nucleic acid encoding the antigenic molecule and may further be transfected with a nucleic acid encoding the hsp or α2M.

In another particularly preferred form of the above embodiments, an hsp or α2M is a recombinant protein.

In another particularly preferred form of the above embodiments, the first and/or second antigen, when present in the composition, is a synthetic or recombinantly generated peptide.

In another particularly preferred form of the above embodiments, the first antigen and/or second antigen, when present in the composition, is bound to an hsp or α2M in vitro.

The present invention also encompasses methods for making pharmaceutical compositions comprising (a) an hsp or α2M, (b) a first antigen that will elicit a specific immune response to a cancer, infectious disease, neurodegenerative or amyloid disease, or autoimmune disease, and (c) a saponin adjuvant, said methods comprising combining the saponin, the first antigen and the hsp or α2M under conditions that produce a pharmaceutically effective composition.

In one embodiment, the saponin is combined with a specific first antigen and then with antigen-free hsp or α2M.

In another embodiment, the saponin is covalently linked with a specific first antigen and then combined with antigen-free hsp or α2M (i.e. stripped of endogenous antigenic molecule). In a preferred embodiment, the antigen covalently linked to the saponin is combined with the antigen-free hsp or α2M under conditions that allow binding of antigen to hsp or α2M.

In another embodiment, antigen-free hsp or α2M is combined with a specific first antigen and then with the saponin. In a preferred embodiment, the specific first antigen is combined with the antigen-free hsp or α2M under conditions that allow binding of antigen to hsp.

In another embodiment, antigen-free hsp or α2M, a saponin, and a specific first antigen are combined simultaneously.

Another embodiment comprises combining aqueous solutions of saponin, antigen, and antigen-free hsp or α2M, under conditions that permit antigen binding to the antigen-free hsp or α2M.

In the compositions of the invention, the presence of saponins does not adversely affect the ability of hsp or α2M to bind or retain noncovalently-bound antigenic molecules.

4.2. Purification of α2M and hsp Complexes

The following α2M or hsp polypeptides stripped of antigenic molecules, and α2M-antigenic molecule complexes or hsp-antigenic molecule complexes, can be used toghether with one or more saponins in the compositions of the invention.

4.2.1. α2M Antigenic Molecule Complexes

Described below are methods for purifying α2M polypeptides or α2M polypeptide-antigenic molecule complexes for use in the invention from recombinant cells, and, with minor modifications known in the art, the α2M polypeptide or α2M-antigenic molecule complexes from cell culture. Recombinant cells include, for example, cells expressing antigenic molecules and recombinantly expressing an α2M polypeptide. Such cells may be derived from a variety of sources, including, but not limited to, cells infected with an infectious agent and cancer cells.

Methods are disclosed for purification of recombinant α2M polypeptide-antigenic molecule complexes by affinity purification, based on the properties of the affinity label present on the α2M polypeptide. One approach is based on specific molecular interactions between a tag and its binding partner. The other approach relies on the immunospecific binding of an antibody to an epitope present on the tag. The principle of affinity chromatography well known in the art is generally applicable to both of these approaches.

To produce α2M polypeptide-antigenic molecule complexes, a nucleotide sequence encoding an α2M polypeptide can be introduced into a cell. When an antigenic molecule is present in the cell, the α2M polypeptide can associate intracellularly with the antigenic molecule, forming a covalent or a non-covalent complex of α2M polypeptide and the antigenic molecule. Cells into which an α2M polypeptide-encoding nucleotide sequence can be introduced, include, but are not limited to, epithelial cells, endothelial cells, eratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc. The choice of cell type depends on the type of tumor, infectious disease or neurodegenerative or amyloid disease being treated or prevented, and can be determined by one of skill in the art.

For example, an expression construct comprising a nucleic acid sequence encoding the α2M polypeptide is introduced into an antigenic cell. As used herein, antigenic cells may include cells that are infected with an infectious agent or pathogen, cells infected with non-infectious or non-pathogenic forms of an infectious agent or pathogen (e.g., by use of a helper infectious agent), tissue or cell culture model systems for neurodegenerative or amyloid diseases, cells infected by or engineered to express an attenuated form of an infectious agent or a non-pathogenic or replication-deficient variant of a pathogen, pre-neoplastic cells that are infected with a cancer-causing infectious agent, such as a virus, but which are not yet neoplastic; or antigenic cells that have been exposed to a mutagen or cancer-causing agent, such as, for example DNA-damaging agents, radiation, etc. Other cells that can be used are pre-neoplastic cells which are in transition from a normal to a neoplastic form as characterized by morphology, physiological or biochemical functions. Preferably, the cancer cells and pre-neoplastic cells used in the methods of the invention are of mammalian origin. Mammals contemplated by this aspect of the invention include humans, companion animals (e.g., dogs and cats), livestock animals (e.g., sheep, cattle, goats, pigs and horses), laboratory animals (e.g., mice, rats and rabbits), and captive or free wild animals.

Virtually any cancer cell, preferably a human cancer cell, can be used in the present methods for producing α2M polypeptide-antigenic molecule complexes. The cancer cells provide the antigenic peptides which become associated covalently or noncovalently with the expressed α2M polypeptide. α2M polypeptide-antigenic molecule complexes are then purified from the cells and used to treat such cancers. Cancers which can be treated or prevented with immunogenic compositions prepared by methods of the invention include, but are not limited to, tumors such as sarcomas and carcinomas. Examples of cancers that are amenable to the methods of the invention are listed in the Target Cancers Section below. Accordingly, any tissues or cells isolated from a pre-neoplastic lesion, a cancer, including cancer that has metastasized to multiple remote sites, can be used in the present method. For example, cells found in abnormally growing tissue, circulating leukemic cells, metastatic lesions as well as solid tumor tissue can be used.

Cell lines derived from a pre-neoplastic lesion, cancer tissues or cancer cells can also be used, provided that the cells of the cell line have at least one or more antigenic determinants in common with antigens on the target cancer cells. Cancer tissues, cancer cells, cells infected with a cancer-causing agent, other pre-neoplastic cells, and cell lines of human origin are preferred.

Cancer and pre-neoplastic cells can be identified by any method known in the art. For example, cancer cells can be identified by morphology, enzyme assays, proliferation assays, cytogenetic characterization, DNA mapping, DNA sequencing, the presence of cancer-causing virus, or a history of exposure to mutagen or cancer-causing agent, imaging, etc. Cancer cells may also be obtained by surgery, endoscopy, or other biopsy techniques. If some distinctive characteristics of the cancer cells are known, they can also be obtained or purified by any biochemical or immunological methods known in the art, such as but not limited to affinity chromatography, and fluorescence activated cell sorting (e.g., with fluorescently tagged antibody against an antigen expressed by the cancer cells).

Cancer tissues, cancer cells or cell lines may be obtained from a single individual or pooled from several individuals. It is not essential that clonal, homogeneous, or purified population of cancer cells be used. It is also not necessary to use cells of the ultimate target in vivo (e.g., cells from the tumor of the intended recipient), so long as at least one or more antigenic determinants on the target cancer cells is present on the cells used for expression of the α2M polypeptide. In addition, cells derived from distant metastases may be used to prepare an immunogenic composition against the primary cancer. A mixture of cells can be used provided that a substantial number of cells in the mixture are cancer cells and share at least one antigenic determinant with the target cancer cell. In a specific embodiment, the cancer cells to be used in expressing an α2M polypeptide are purified.

4.2.1.1 In vitro Complexing

Complexes of α2M polypeptides and antigenic molecules may be produced in vitro. Immunogenic α2M polypeptide-antigenic molecule complexes can be generated in vitro by coupling of an α2M polypeptide with an antigenic peptide. Procedures for forming such α2M-antigenic molecule complexes and methods for isolating antigenic peptides are described below.

In general, when an α2M is mixed with a protease, cleavage of the “bait” region of α2M takes place, the proteinase becomes “trapped” by thioesters, and a conformational change takes place that allows binding of the α2M complex to the α2M receptor. During proteolytic activation of α2M, non-proteolytic ligands can become covalently bound to the activated thioesters. Non-proteolytic ligands can also be incorporated into the activated α2M molecule by ammonia or methylamine during reversal of the nucleophilic activation, employing heat (Grøn and Pizzo, 1998, Biochemistry, 37: 6009-6014). Such conditions that allow fortuitous trapping of peptides by α2M are employed to prepare the α2M -antigenic complexes for use in the invention. Methods for such covalent coupling have been described previously (Osada et al., 1987, Biochem. Biophys. Res. Commun.146:26-31; Osada et al., 1988, Biochem. Biophys. Res. Commun. 150:883; Chu and Pizzo, 1993, J. Immunol. 150:48; Chu et al., 1994, Ann. N.Y. Acad. Sci. 737:291-307; Mitsuda et al., 1993, Biochem. Biophys. Res. Commun. 101:1326-1331). Thus in one embodiment, an α2M antigenic molecule complex can be prepared as described by Grøn and Pizzo, 1998, Biochemistry, 37: 6009-6014. The method of Grøn and Pizzo yields complexes of α2M that are covalently bound to antigenic molecules.

For example, α2M polypeptide is mixed with an antigenic molecule in the presence of a protease, ammonia or other small amine nucleophiles such as methylamine and ethylamine. Non-limiting examples of proteases which may be used include trypsin, porcine pancreatic elastase (PEP), human neutrophil elastase, cathepsin G, S. aureus V-8 proteinase trypsin, α-chymotrypsin, V8 protease, papain, and proteinase K (see Ausubel et al., eds., in “Current Protocols in Molecular Biology”, Greene Publishing Associates and Wiley Interscience, New York, 17.4.6-17.4.8). A preferred, exemplary protocol for complexing an α2M polypeptide and an antigenic molecule in vitro follows. The antigenic molecules (1 μg-20 mg) and the α2M polypeptide (1 μg-20 mg) are mixed together in phosphate-buffered saline (PBS) (100 μl-5 ml) in the presence of a protease, such as trypsin (0.92 mg trypsin in approximately 500 μl PBS, to give an approximately 5:1 antigenic molecule:α2M polypeptide molar ratio. The mixture is then incubated for 5-15 minutes at 37° C. 500 μl 4 mg/ml p-Aphenyl methyl sulfonyl fluoride (p-APMSF) is added to the solution to inhibit trypsin activity and incubated for 2 hrs at 25° C. The preparations can be centrifuged through a Centricon 10 assembly (Millipore) to remove any unbound peptide. Alternatively, free antigenic molecule may be removed by passage over a gel permeation column. The association of the peptides with the α2M polypeptide can be assayed by SDS-PAGE. This is the preferred method for in vitro complexing of antigenic molecules isolated from MHC-antigenic molecule complexes, or peptides disassociated from endogenous α2M-antigenic molecule complexes.

In a more preferred method, an α2M-antigenic molecule complex is prepared according to the method described by Blachere et al., J. Exp. Med. 186(8):1315-22, which incorporated by reference herein in its entirety for in vitro complexing of hsps to antigenic molecule, with the hsp component substituted by α2M. The Blachere method yields complexes of α2M bound to antigenic molecules.

Antigenic molecules may be isolated from various sources, chemically synthesized, or produced recombinantly. Such methods can be readily adapted for medium or large scale production of the immunotherapeutic or prophylactic vaccines.

Following complexing, the immunogenic α2M-antigenic molecule complexes can optionally be assayed in vitro using, for example, the mixed lymphocyte target cell assay (MLTC) described below. Once immunogenic complexes have been isolated they can be optionally characterized further in animal models using the preferred administration protocols and excipients discussed below.

4.2.2. Preparation and Purification of hsp70-peptide Complexes

The purification of hsp70-peptide complexes has been described previously, see, for example, Udono et al., 1993, J. Exp. Med. 178:1391-1396. A procedure that may be used, presented by way of example but not limitation, is as follows:

Initially, tumor cells are suspended in 3 volumes of 1×Lysis buffer consisting of 5 mM sodium phosphate buffer, pH 7, 150 mM NaCl, 2 mM CaCl₂, 2 mM MgCl₂ and 1 mM phenyl methyl sulfonyl fluoride (PMSF). Then, the pellet is sonicated, on ice, until >99% cells are lysed as determined by microscopic examination. As an alternative to sonication, the cells may be lysed by mechanical shearing and in this approach the cells typically are resuspended in 30 mM sodium bicarbonate pH 7.5, 1 mM PMSF, incubated on ice for 20 minutes and then homogenized in a Dounce homogenizer until >95% cells are lysed.

Then the lysate is centrifuged at 1,000 g for 10 minutes to remove unbroken cells, nuclei and other cellular debris. The resulting supernatant is recentrifuged at 100,000 g for 90 minutes, the supernatant harvested and then mixed with Con A Sepharose equilibrated with phosphate buffered saline (PBS) containing 2 mM Ca²⁺ and 2 mM Mg²⁺. When the cells are lysed by mechanical shearing the supernatant is diluted with an equal volume of 2×lysis buffer prior to mixing with Con A Sepharose. The supernatant is then allowed to bind to the Con A Sepharose for 2-3 hours at 4° C. The material that fails to bind is harvested and dialyzed for 36 hours (three times, 100 volumes each time) against 10 mM Tris-Acetate pH 7.5, 0.1 mM EDTA, 10 mM NaCl, 1 mM PMSF. Then the dialyzate is centrifuged at 17,000 rpm (e.g. Sorvall SS34 rotor) for 20 minutes. Then the resulting supernatant is harvested and applied to a Mono Q FPLC column equilibrated in 20 mM Tris-Acetate pH 7.5, 20 mM NaCl, 0.1 mM EDTA and 15 mM 2-mercaptoethanol. The column is then developed with a 20 mM to 500 mM NaCl gradient and then eluted fractions fractionated by sodium dodecyl sulfate-polyacrylamide gel electrophoresis (SDS-PAGE) and characterized by immunoblotting using an appropriate anti-hsp70 antibody (such as from clone N27F3-4, from StressGen).

Fractions strongly immunoreactive with the anti-hsp70 antibody are pooled and the hsp70-peptide complexes precipitated with ammonium sulfate; specifically with a 50%-70% ammonium sulfate cut. The resulting precipitate is then harvested by centrifugation at 17,000 rpm (SS34 Sorvall rotor) and washed with 70% ammonium sulfate. The washed precipitate is then solubilized and any residual ammonium sulfate removed by gel filtration on a Sephadex^{R} G25 column (Pharmacia). If necessary the hsp70 preparation thus obtained can be repurified through the Mono Q FPLC Column as described above.

The hsp70-peptide complex can be purified to apparent homogeneity using this method. Typically 1 mg of hsp70-peptide complex can be purified from 1 g of cells/tissue.

An improved method for purification of hsp70-peptide complexes comprises contacting cellular proteins with ADP or a nonhydrolyzable analog of ATP affixed to a solid substrate, such that hsp70 in the lysate can bind to the ADP or nonhydrolyzable ATP analog, and eluting the bound hsp70. A preferred method uses column chromatography with ADP affixed to a solid substratum (e.g., ADP-agarose). The resulting hsp70 preparations are higher in purity and devoid of contaminating peptides. The hsp70 yields are also increased significantly by about more than 10 fold. Alternatively, chromatography with nonhydrolyzable analogs of ATP, instead of ADP, can be used for purification of hsp70-peptide complexes. By way of example but not limitation, purification of hsp70-peptide complexes by ADP-agarose chromatography can be carried out as follows:

Meth A sarcoma cells (500 million cells) are homogenized in hypotonic buffer and the lysate is centrifuged at 100,000 g for 90 minutes at 4° C. The supernatant is applied to an ADP-agarose column. The column is washed in buffer and is eluted with 5 column volumes of 3 mM ADP. The hsp70-peptide complexes elute in fractions 2 through 10 of the total 15 fractions which elute. The eluted fractions are analyzed by SDS-PAGE. The hsp70-peptide complexes can be purified to apparent homogeneity using this procedure.

4.2.3. Preparation and Purification of hsp90-peptide Complexes

A procedure that can be used, presented by way of example and not limitation, is as follows:

Initially, tumor cells are suspended in 3 volumes of 1×Lysis buffer consisting of 5 mM sodium phosphate buffer (pH7), 150 mM NaCl, 2 mM CaCl₂, 2 mM MgCl₂ and 1 mM phenyl methyl sulfonyl fluoride (PMSF). Then, the pellet is sonicated, on ice, until >99% cells are lysed as determined by microscopic examination. As an alternative to sonication, the cells may be lysed by mechanical shearing and in this approach the cells typically are resuspended in 30 mM sodium bicarbonate pH 7.5, 1 mM PMSF, incubated on ice for 20 minutes and then homogenized in a Dounce homogenizer until >95% cells are lysed.

Then the lysate is centrifuged at 1,000 g for 10 minutes to remove unbroken cells, nuclei and other cellular debris. The resulting supernatant is recentrifuged at 100,000 g for 90 minutes, the supernatant harvested and then mixed with Con A Sepharose equilibrated with PBS containing 2 mM Ca²⁺ and 2 mM Mg²⁺. When the cells are lysed by mechanical shearing the supernatant is diluted with an equal volume of 2×Lysis buffer prior to mixing with Con A Sepharose. The supernatant is then allowed to bind to the Con A Sepharose for 2-3 hours at 4° C. The material that fails to bind is harvested and dialyzed for 36 hours (three times, 100 volumes each time) against 10 mM Tris-Acetate pH 7.5, 0.1 mM EDTA, 10 mM NaCl, 1 mM PMSF. Then the dialyzate is centrifuged at 17,000 rpm (Sorvall SS34 rotor) for 20 minutes. Then the resulting supernatant is harvested and applied to a Mono Q FPLC column equilibrated with lysis buffer. The proteins are then eluted with a salt gradient of 200 mM to 600 mM NaCl.

The eluted fractions are fractionated by SDS-PAGE and fractions containing the hsp90-peptide complexes identified by immunoblotting using an anti-hsp90 antibody such as 3G3 (Affinity Bioreagents). hsp90-peptide complexes can be purified to apparent homogeneity using this procedure. Typically, 150-200 μg of hsp90-peptide complex can be purified from 1 g of cells/tissue.

4.2.4. Preparation and Purification of gp96-peptide Complexes

A procedure that can be used, presented by way of example and not limitation, is as follows:

A pellet of tumors is resuspended in 3 volumes of buffer consisting of 30 mM sodium bicarbonate buffer (pH 7.5) and 1 mM PMSF and the cells allowed to swell on ice 20 minutes. The cell pellet is then homogenized in a Dounce homogenizer (the appropriate clearance of the homogenizer will vary according to each cell type) on ice until >95% cells are lysed.

The lysate is centrifuged at 1,000 g for 10 minutes to remove unbroken cells, nuclei and other debris. The supernatant from this centrifugation step is then recentrifuged at 100,000 g for 90 minutes. The gp96-peptide complex can be purified either from the 100,000 pellet or from the supernatant.

When purified from the supernatant, the supernatant is diluted with equal volume of 2×lysis buffer and the supernatant mixed for 2-3 hours at 4° C. with Con A Sepharose equilibrated with PBS containing 2 mM Ca²⁺ and 2 mM Mg²⁺. Then, the slurry is packed into a column and washed with 1×lysis buffer until the OD₂₈₀ drops to baseline. Then, the column is washed with ⅓ column bed volume of 10% α-methyl mannoside (α-MM) dissolved in PBS containing 2 mM Ca²⁺ and 2 mM Mg²⁺, the column sealed with a piece of parafilm, and incubated at 37° C. for 15 minutes. Then the column is cooled to room temperature and the parafilm removed from the bottom of the column. Five column volumes of the α-MM buffer are applied to the column and the eluate analyzed by SDS-PAGE. Typically the resulting material is about 60-95% pure, however this depends upon the cell type and the tissue-to-lysis buffer ratio used. Then the sample is applied to a Mono Q FPLC column (Pharmacia) equilibrated with a buffer containing 5 mM sodium phosphate, pH 7. The proteins are then eluted from the column with a 0-1M NaCl gradient and the gp96 fraction elutes between 400 mM and 550 mM NaCl.

The procedure, however, may be modified by two additional steps, used either alone or in combination, to consistently produce apparently homogeneous gp96-peptide complexes. One optional step involves an ammonium sulfate precipitation prior to the Con A purification step and the other optional step involves DEAE-Sepharose purification after the Con A purification step but before the Mono Q FPLC step.

In the first optional step, described by way of example as follows, the supernatant resulting from the 100,000 g centrifugation step is brought to a final concentration of 50% ammonium sulfate by the addition of ammonium sulfate. The ammonium sulfate is added slowly while gently stirring the solution in a beaker placed in a tray of ice water. The solution is stirred from about ½ to 12 hours at 4° C. and the resulting solution centrifuged at 6,000 rpm (Sorvall SS34 rotor). The supernatant resulting from this step is removed, brought to 70% ammonium sulfate saturation by the addition of ammonium sulfate solution, and centrifuged at 6,000 rpm (Sorvall SS34 rotor). The resulting pellet from this step is harvested and suspended in PBS containing 70% ammonium sulfate in order to rinse the pellet. This mixture is centrifuged at 6,000 rpm (Sorvall SS34 rotor) and the pellet dissolved in PBS containing 2 mM Ca²⁺ and Mg²⁺. Undissolved material is removed by a brief centrifugation at 15,000 rpm (Sorvall SS34 rotor). Then, the solution is mixed with Con A Sepharose and the procedure followed as before.

In the second optional step, described by way of example as follows, the gp96 containing fractions eluted from the Con A column are pooled and the buffer exchanged for 5 mM sodium phosphate buffer, pH 7, 300 mM NaCl by dialysis, or preferably by buffer exchange on a Sephadex G25 column. After buffer exchange, the solution is mixed with DEAE-Sepharose previously equilibrated with 5 mM sodium phosphate buffer, pH 7, 300 mM NaCl. The protein solution and the beads are mixed gently for 1 hour and poured into a column. Then, the column is washed with 5 mM sodium phosphate buffer, pH 7, 300 mM NaCl, until the absorbance at 280 nm drops to baseline. Then, the bound protein is eluted from the column with five volumes of 5 mM sodium phosphate buffer, pH 7, 700 mM NaCl. Protein containing fractions are pooled and diluted with 5 mM sodium phosphate buffer, pH 7 in order to lower the salt concentration to 175 mM. The resulting material then is applied to the Mono Q FPLC column (Pharmacia) equilibrated with 5 mM sodium phosphate buffer, pH 7 and the protein that binds to the Mono Q FPLC column (Pharmacia) is eluted as described before.

It is appreciated, however, that one skilled in the art may assess, by routine experimentation, the benefit of incorporating the second optional step into the purification protocol. In addition, it is appreciated also that the benefit of adding each of the optional steps will depend upon the source of the starting material.

When the gp96 fraction is isolated from the 100,000 g pellet, the pellet is suspended in 5 volumes of PBS containing either 1% sodium deoxycholate or 1% oxtyl glucopyranoside (but without the Mg²⁺ and Ca²⁺) and incubated on ice for 1 hour. The suspension is centrifuged at 20,000 g for 30 minutes and the resulting supernatant dialyzed against several changes of PBS (also without the Mg²⁺ and Ca²⁺) to remove the detergent. The dialysate is centrifuged at 100,000 g for 90 minutes, the supernatant harvested, and calcium and magnesium are added to the supernatant to give final concentrations of 2 mM, respectively. Then the sample is purified by either the unmodified or the modified method for isolating gp96-peptide complex from the 100,000 g supernatant, see above.

The gp96-peptide complexes can be purified to apparent homogeneity using this procedure. About 10-20 μg of gp96 can be isolated from 1 g cells/tissue.

4.2.5. Preparation and Purification of hsp110-peptide Complexes

A procedure, described by Wang et al., 2001, J. Immunol. 166(1):490-7, that can be used, presented by way of example and not limitation, is as follows:

A pellet (40-60 ml) of cell or tissue, e.g., tumor cell tissue, is homogenized in 5 vol of hypotonic buffer (30 mN sodium bicarbonate, pH7.2, and protease inhibitors) by Dounce homogenization. The lysate is centrifuged at 4,500×g and then 100,000×g for 2 hours. If the cells or tissues are of hepatic origin, the resulting supernatant is was first applied to a blue Sepharose column (Pharmacia) to remove albumin. Otherwise, the resulting supernatant is applied to a Con A-Sepharose column (Pharmacia Biotech, Piscataway, N.J.) previously equilibrated with binding buffer (20 mM Tris-HCI, pH 7.5; 100 mM NaCl; 1 mM MgCl₂; 1 mM CaCl₂; 1 mM MnCl₂; and 15 mM 2-ME). The bound proteins are eluted with binding buffer containing 15% α-D-o-methylmannoside (Sigma, St. Louis, Mo.).

Con A-Sepharose unbound material is first dialyzed against a solution of 20 mM Tris-HCl, pH 7.5; 100 mM NaCl; and 15 mM 2-ME, and then applied to a DEAE-Sepharose column and eluted by salt gradient from 100 to 500 mM NaCl. Fractions containing hsp110 are collected, dialyzed, and loaded onto a Mono Q (Pharmacia) 10/10 column equilibrated with 20 mM Tris-HCl, pH 7.5; 200 mM NaCl; and 15 mM 2-ME. The bound proteins are eluted with a 200-500 mM NaCl gradient. Fractions are analyzed by SDS-PAGE followed by immunoblotting with an Ab for hsp110, as described by Wang et al., 1999, J. Immunol. 162:3378. Pooled fractions containing hsp110 are concentrated by Centriplus (Amicon, Beverly, Mass.) and applied to a Superose 12 column (Pharmacia). Proteins are eluted by 40 mM Tris-HCl, pH 8.0; 150 mM NaCl; and 15 mM 2-ME with a flow rate of 0.2 ml/min.

4.2.6. Preparation and Purification of grp170-peptide Complexes

A procedure, described by Wang et al., 2001, J. Immunol. 166(1):490-7, that can be used, presented by way of example and not limitation, is as follows:

A pellet (40-60 ml) of cell or tissue, e.g., tumor cell tissue, is homogenized in 5 vol of hypotonic buffer (30 mN sodium bicarbonate, pH7.2, and protease inhibitors) by Dounce homogenization. The lysate is centrifuged at 4,500×g and then 100,000×g for 2 hours. If the cells or tissues are of hepatic origin, the resulting supernatant is was first applied to a blue Sepharose column (Pharmacia) to remove albumin. Otherwise, the resulting supernatant is applied to a Con A-Sepharose column (Pharmacia Biotech, Piscataway, N.J.) previously equilibrated with binding buffer (20 mM Tris-HCI, pH 7.5; 100 mM NaCl; 1 mM MgCl₂; 1 mM CaCl₂; 1 mM MnCl₂; and 15 mM 2-ME). The bound proteins are eluted with binding buffer containing 15% α-D-o-methylmannoside (Sigma, St. Louis, Mo.).

Con A-Sepharose-bound material is first dialyzed against 20 mM Tris-HCl, pH 7.5, and 150 mM NaCl and then applied to a Mono Q column and eluted by a 150 to 400 mM NaCl gradient. Pooled fractions are concentrated and applied on the Superose 12 column (Pharmacia). Fractions containing homogeneous grp170 are collected.

4.2.7. Infectious Disease

In an alternative embodiment wherein it is desired to treat a patient having an infectious disease, the above-described methods are used to isolate α2M or hsp -antigenic molecule complexes from cells infected with an infectious organism, e.g., of a cell line or from a patient. Such infectious organisms include but are not limited to, viruses, bacteria, protozoa, fungi, and parasites as described in detail in the Target Infectious Diseases Section below.

4.2.8. Autoimmune Disease

In an another alternative embodiment wherein it is desired to treat a patient having an autoimmune disease, the above-described methods are used to isolate α2M or hsp-antigenic molecule complexes from an autologous, allogeneic, or other cell line expressing an antigenic molecules of interest.

4.2.9. Neurodegenerative and Amyloid Diseases

In an another alternative embodiment wherein it is desired to treat a patient having a neurodegenerative or amyloid disease, the above-described methods are used to isolate α2M or hsp-antigenic molecule complexes from an autologous, allogeneic, or other cell line expressing an antigenic molecules of interest. Examples of suitable cells and tissues from which such hsp-antigenic molecule complexes can be isolated are described in U.S. patent application Ser. No. 09/489,215, which is incorporated by reference herein in its entirety.

4.2.10. Peptides from α2M or hsp-Peptide Complexes

Antigenic molecules (e.g. peptides) can be eluted from hsp-antigenic molecule complexes either in the presence of ATP or low pH. Antigenic molecules can be eluted from α2M-antigenic molecule complexes in the presence of low pH. These experimental conditions may be used to isolate peptides or non-peptide antigenic components from cells which may contain potentially useful antigenic determinants. Once isolated, the amino acid sequence of an antigenic peptide may be determined using conventional amino acid sequencing methodologies. Antigenic molecules can then be produced by chemical synthesis or recombinant methods, purified, and complexed to hsps or α2M in vitro.

Thus, potentially immunogenic or antigenic peptides may be isolated from either endogenous stress protein-peptide complexes or endogenous MHC-peptide complexes for use subsequently as antigenic molecules, by complexing in vitro to hsps.

Briefly the complex of interest is centrifuged through a Centricon 10 assembly (Millipore) to remove any low molecular weight material loosely associated with the complex. The large molecular weight fraction may be removed and analyzed by SDS-PAGE while the low molecular weight may be analyzed by HPLC as described below. In the ATP incubation protocol, the stress protein-peptide complex in the large molecular weight fraction is incubated with 10 mM ATP for 30 minutes at room temperature. In the low pH protocol, acetic acid or trifluoroacetic acid (TFA) is added to the stress protein peptide complex to give a final concentration of 10% (vol/vol) and the mixture incubated at room temperature or in a boiling water bath or any temperature in between, for 10 minutes (See, Udono et al., 1993, J. Exp. Med. 178:1391-1396; Van Bleek et al., 1990, Nature 348:213-216; and Li et al., 1993, EMBO Journal 12:3143-3151).

The resulting samples are centrifuged through a Centricon 10 assembly as mentioned previously. The high and low molecular weight fractions are recovered. The remaining large molecular weight stress protein-peptide complexes can be reincubated with ATP or low pH to remove any remaining peptides.

The resulting lower molecular weight fractions are pooled, concentrated by evaporation and dissolved in 0.1% TFA. The dissolved material is then fractionated by reverse phase high pressure liquid chromatography (HPLC) using for example a VYDAC C18 reverse phase column equilibrated with 0.1% TFA. The bound material is then eluted at a flow rate of about 0.8 ml/min by developing the column with a linear gradient of 0 to 80% acetonitrile in 0.1% TFA. The elution of the peptides can be monitored by OD₂₁₀ and the fractions containing the peptides collected.

4.2.11. Peptides from MHC-Peptide Complexes

The isolation of potentially immunogenic peptides from MHC molecules is well known in the art and so is not described in detail herein (See, Falk et al., 1990, Nature 348:248-251; Rotzsche et al., 1990, Nature 348:252-254; Elliott et al., 1990, Nature 348:191-197; Falk et al., 1991, Nature 351:290-296; Demotz et al., 1989, Nature 343:682-684; Rotzsche et al., 1990, Science 249:283-287, the disclosures of which are incorporated herein by reference).

Briefly, MHC-peptide complexes may be isolated by a conventional immunoaffinity procedure. The peptides then may be eluted from the MHC-peptide complex by incubating the complexes in the presence of about 0.1% TFA in acetonitrile. The eluted peptides may be fractionated and purified by reverse phase HPLC, as before.

The amino acid sequences of the eluted peptides may be determined either by manual or automated amino acid sequencing techniques well known in the art. Once the amino acid sequence of a potentially protective peptide has been determined the peptide may be synthesized in any desired amount using conventional peptide synthesis or other protocols well known in the art.

Peptides having the same amino acid sequence as those isolated above may be synthesized by solid-phase peptide synthesis using procedures similar to those described by Merrifield, 1963, J. Am. Chem. Soc. 85:2149. During synthesis, N-α-protected amino acids having protected side chains are added stepwise to a growing polypeptide chain linked by its C-terminal and to an insoluble polymeric support i.e., polystyrene beads. The peptides are synthesized by linking an amino group of an N-α-deprotected amino acid to an α-carboxy group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc which is acid labile and Fmoc which is base labile.

Briefly, the C-terminal N-α-protected amino acid is first attached to the polystyrene beads. The N-α-protecting group is then removed. The deprotected α-amino group is coupled to the activated α-carboxylate group of the next N-α-protected amino acid. The process is repeated until the desired peptide is synthesized. The resulting peptides are then cleaved from the insoluble polymer support and the amino acid side chains deprotected. Longer peptides can be derived by condensation of protected peptide fragments. Details of appropriate chemistries, resins, protecting groups, protected amino acids and reagents are well known in the art and so are not discussed in detail herein (See, Atherton et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting peptides is accomplished using conventional procedures, such as preparative HPLC using gel permeation, partition and/or ion exchange chromatography. The choice of appropriate matrices and buffers are well known in the art and so are not described in detail herein.

4.2.12. Exogenous Antigenic Molecules

Any antigen, or an antigenic portion thereof, can be selected for use as an antigenic molecule for complexing to hsps, MHCs, or α2M. For example, antigens may be selected from among those known in the art or determined by immunoassay to be able to bind to antibody or MHC molecules (antigenicity) or generate an immune response (immunogenicity). To determine immunogenicity or antigenicity by detecting binding to antibody, various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in vivo immunoassays (using colloidal gold, enzyme or radioisotope labels, for example), western blots, immunoprecipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labelled. Many means are known in the art for detecting binding in an immunoassay and are envisioned for use. In one embodiment for detecting immunogenicity, T cell-mediated responses can be assayed by standard methods, e.g., in vitro cytoxicity assays or in vivo delayed-type hypersensitivity assays.

Potentially useful antigens or derivatives thereof for use as antigenic molecules can also be identified by various criteria, such as the antigen's involvement in neutralization of a pathogen's infectivity (wherein it is desired to treat or prevent infection by such a pathogen) (Norrby, 1985, Summary, in Vaccines 85, Lerner et al. (eds.), Cold Spring Harbor Laboratory, Cold Spring Harbor, N.Y., pp. 388-389), type or group specificity, recognition by patients' antisera or immune cells, and/or the demonstration of protective effects of antisera or immune cells specific for the antigen. In addition, where it is desired to treat or prevent a disease caused by pathogen, the antigen's encoded epitope should preferably display a small or no degree of antigenic variation in time or amongst different isolates of the same pathogen.

Preferably, where it is desired to treat or prevent cancer, known tumor-specific antigens or fragments or derivatives thereof are used. For example, such tumor specific or tumor-associated antigens include but are not limited to KS ¼ pan-carcinoma antigen (Perez and Walker, 1990, J. Immunol. 142:3662-3667; Bumal, 1988, Hybridoma 7(4):407-415); ovarian carcinoma antigen (CA125) (Yu et al., 1991, Cancer Res. 51(2):468-475); prostatic acid phosphate (Tailer et al., 1990, Nucl. Acids Res. 18(16):4928); prostate specific antigen (Henttu and Vihko, 1989, Biochem. Biophys. Res. Comm. 160(2):903-910; Israeli et al., 1993, Cancer Res. 53:227-230); melanoma-associated antigen p97 (Estin et al., 1989, J. Natl. Cancer Inst. 81(6):445-446); melanoma antigen gp75 (Vijayasardahl et al., 1990, J. Exp. Med. 171(4):1375-1380); high molecular weight melanoma antigen (Natali et al., 1987, Cancer 59:55-63) and prostate specific membrane antigen.

In a specific embodiment, an antigen or fragment or derivative thereof specific to a certain tumor is selected for complexing to hsp and subsequent administration to a patient having that tumor.

Preferably, where it is desired to treat or prevent viral diseases, molecules comprising epitopes of known viruses are used. For example, such antigenic epitopes may be prepared from viruses including, but not limited to, hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus, polio virus, human immunodeficiency virus type I (HIV-I), and human immunodeficiency virus type II (HIV-II).

Preferably, where it is desired to treat or prevent bacterial infections, molecules comprising epitopes of known bacteria are used. For example, such antigenic epitopes may be prepared from bacteria including, but not limited to, mycobacteria rickettsia, mycoplasma, neisseria and legionella.

Preferably, where it is desired to treat or prevent protozoal infections, molecules comprising epitopes of known protozoa are used. For example, such antigenic epitopes may be prepared from protozoa including, but not limited to, leishmania, kokzidioa, and trypanosoma.

Preferably, where it is desired to treat or prevent parasitic infections, molecules comprising epitopes of known parasites are used. For example, such antigenic epitopes may be from parasites including, but not limited to, chlamydia and rickettsia.

Preferably, where it is desired to treat or prevent neurodegenerative or amyloid diseases, molecules comprising epitopes of antigenic molecules associated with neurodegenerative diseases, or epitopes of antigenic molecules associated with amyloid diseases, including but not limited to fibril peptides or proteins, are used. For example, such neurodegenerative disease-associated antigenic molecules may be molecules associated with Alzheimer's Disease, age-related loss of cognitive function, senile dementia, Parkinson's disease, amyotrophic lateral sclerosis, Wilson's Disease, cerebral palsy, progressive supranuclear palsy, Guam disease, Lewy body dementia, prion diseases, spongiform encephalopathies, Creutzfeldt-Jakob disease, polyglutamine diseases, Huntington's disease, myotonic dystrophy, Freidrich's ataxia, ataxia, Gilles de la Tourette's syndrome, seizure disorders, epilepsy, chronic seizure disorder, stroke, brain trauma, spinal cord trauma, AIDS dementia, alcoholism, autism, retinal ischemia, glaucoma, autonomic function disorder, hypertension, neuropsychiatric disorder, schizophrenia, or schizoaffective disorder. Antigenic molecules that are suitable for in vitro complexing methods are disclosed in U.S. application Ser. No. 09/489,216, which is incorporated by reference herein in its entirety, and include, but are not limited to, β-amyloid, an oligomeric Aβ complex, an ApoE4-Aβ complex, tau protein, a mutant amyloid precursor, a mutant of presenillin, α-synuclein, a prion protein, or an antigenic fragment of any of the foregoing proteins. Amyloid disease associated antigenic molecules may be molecules associated with diseases characterized by the extracellular deposition of protein and/or peptide fibrils which form amyloid deposits or plaques, including but not limited to type II diabetes and amyloidoses associated with chronic inflammatory or infectious disease states and malignant neoplasms, e.g., myeloma. Certain amyloid disease such as Alzheimer's disease and prion diseases, e.g., Creutzfeldt Jacob disease, are neurodegenerative diseases.

4.2.13. In vitro Production of hsp-Antigenic Molecule Complexes

In an embodiment in which complexes of hsps and the peptides with which they are endogenously associated in vivo are not employed, complexes of hsps to antigenic molecules are produced in vitro. As will be appreciated by those skilled in the art, the peptides either isolated by the aforementioned procedures or chemically synthesized or recombinantly produced may be reconstituted with a variety of purified natural or recombinant stress proteins in vitro to generate immunogenic non-covalent stress protein-antigenic molecule complexes. Alternatively, exogenous antigens or antigenic or immunogenic fragments or derivatives thereof can be complexed to stress proteins for use in the immunotherapeutic or prophylactic vaccines of the invention. A preferred, exemplary protocol for complexing a stress protein and an antigenic molecule in vitro is discussed below.

Prior to complexing, the hsps are pretreated with ATP or low pH to remove any peptides that may be associated with the hsp of interest. When the ATP procedure is used, excess ATP is removed from the preparation by the addition of apyranase as described by Levy et al., 1991, Cell 67:265-274. When the low pH procedure is used, the buffer is readjusted to neutral pH by the addition of pH modifying reagents.

The antigenic molecules (1 μg) and the pretreated hsp (9 μg) are admixed to give an approximately 5 antigenic molecule: 1 stress protein molar ratio. Then, the mixture is incubated for 15 minutes to 3 hours at 4° to 45° C. in a suitable binding buffer such as one containing 20 mM sodium phosphate, pH 7.2, 350 mM NaCl, 3 mM MgCl₂ and 1 mM phenyl methyl sulfonyl fluoride (PMSF). The preparations are centrifuged through a Centricon 10 assembly (Millipore) to remove any unbound peptide. The association of the peptides with the stress proteins can be assayed by SDS-PAGE. This is the preferred method for in vitro complexing of peptides isolated from MHC-peptide complexes of peptides disassociated from endogenous hsp-peptide complexes.

In an alternative embodiment of the invention, preferred for producing complexes of hsp70 to exogenous antigenic molecules such as proteins, 5-10 micrograms of purified hsp is incubated with equimolar quantities of the antigenic molecule in 20 mM sodium phosphate buffer pH 7.5, 0.5M NaCl, 3 mM MgCl₂ and 1 mM ADP in a volume of 100 microliter at 37° C. for 1 hr. This incubation mixture is further diluted to 1 ml in phosphate-buffered saline.

In an alternative embodiment of the invention, preferred for producing complexes of gp96 or hsp90 to peptides, 5-10 micrograms of purified gp96 or hsp90 is incubated with equimolar or excess quantities of the antigenic peptide in a suitable buffer such as one containing 20 mM sodium phosphate buffer pH 7.5, 0.5M NaCl, 3 nM MgCl2 at 60-65° C. for 5-20 min. This incubation mixture is allowed to cool to room temperature and centrifuged one or more times if necessary, through a Centricon 10 assembly (Millipore) to remove any unbound peptide.

Following complexing, the immunogenic stress protein-antigenic molecule complexes can optionally be assayed in vitro using for example the mixed lymphocyte target cell assay (MLTC) described below. Once immunogenic complexes have been isolated they can be optionally characterized further in animal models using the preferred administration protocols and excipients discussed below.

4.3. Sources of Saponins

Any saponin or saponin preparation known in the art may be used in the compositions and methods of the invention. The term “saponin” as used herein includes glycosidic triterpenoid compounds which produce foam in aqueous solution and have hemolytic activity in most cases. The invention encompasses the use of saponins per se, as well as natural and pharmaceutically acceptable salts and pharmaceutically acceptable derivatives thereof. The term “saponin” also embodies biologically active fragments thereof. The term “saponin” also encompasses chemically modified saponins. In other embodiments of the invention, the term “saponin” covers mixtures of saponins. Preferably, the mixture of saponins comprises two or more substantially pure saponins. More preferably, the two or more substantially pure saponins are from Quillaja saponaria in doses that are otherwise suboptimal for the individual saponins. In a particularly preferred embodiment, the combination of saponins consists essentially of two or substantially pure saponins QS-7 and QS-21 or, in other particularly preferred embodiments, QS-7 and QS-21-V1 or QS-7 and QS-21-V2, as described in U.S. Pat. No. 6,231,859, which is herein incorporated by reference in its entirety. As used herein, “substantially pure” means substantially free from compounds normally associated with the saponin in its natural state and exhibiting constant and reproducible chromatographic response, elution profiles, and biologic activity. The term “substantially pure” is not meant to exclude artificial or synthetic mixtures of the saponin with other compounds. A number of non-limiting examples of saponins and their methods of preparation are provided below.

Several embodiments of the invention relate to pharmaceutical compositions comprising either α2M or an hsp, a first antigen that will elicit a specific immune response to a cancer, infectious disease or neurodegenerative or amyloid disease, and a saponin adjuvant. The compositions of the invention comprising saponins can be formulated in a variety of ways to produce pharmaceutically effective compositions capable of eliciting an immune response in an individual to whom the composition is administered.

Additional embodiments of the invention relate to pharmaceutical compositions comprising either α2M or an hsp, optionally a peptide (which need not be antigenic), and a saponin adjuvant, for the prevention or treatment of an autoimmune disorder. These compositions are formulated to be capable of enhancing the suppression of an immune response in an individual to whom the composition is administered.

Saponins suitable for use in an individual are soluble in aqueous solution and can be reconstituted from lyophilized or dried saponins. Specific saponins useful for the present invention include, but are not limited to, the “Quil-A” adjuvant preparation sold by Superfos of Norway, and the chromatographic fractions with adjuvant activity that are described in U.S. Pat. Nos. 5,057,540 and 5,583,112, particularly fractions QS-21 (also referred to in the patents as QA-21) and QS-7.

Also useful in the methods and compositions of the present invention are chemically modified saponins that retain adjuvant activity. According to Kensil et al., U.S. Pat. No. 5,583,112, the contents of which are fully incorporated by reference herein, the carboxyl group on the glucuronic acid of saponins from Quillaja saponaria Molina can be conjugated to a protein, a peptide, or a small molecule containing a primary amine. According to Higuchi et al., 1987, Phytochemistry 26:229, saponins from Quillaja saponaria may be deacylated by alkaline-catalyzed hydrolysis. According to Marciani et al., U.S. Pat. No. 5,977,081, the contents of which are fully incorporated by reference herein, the carboxyl group on the glucuronic acid of nonacylated or deacylated saponins from Quillaja saponaria may be conjugated to a lipid, fatty acid, polyethylene glycol, or terpene.

Alternatively, an active fragment or synthetically modified derivative of a fragment or a native saponin(s) may be utilized, such as those described in Soltysik et al., 1995, Vaccine 13(15):1403-1410; Marciani et al., 2000, Vaccine 18:3141-3151. Such modifications include but are not limited to removals or substitutions of saccharide residues, addition of saccharide residues, and removal, substitution and/or addition of acyl chains.

The methods and compositions of the present invention may also employ saponins isolated from plant species other than Quillaja, such as Gypsophila or Saponaria officinalis.

In certain embodiments of the invention, compositions of the invention comprises saponins in combination with excipients. Preferably, the saponin is QS-21 and the excipients are selected from nonionic surfactants, polyvinyl pyrolidone, human serum albumin, and various unmodified and derivatized cyclodextrins. More preferably, in these embodiments, the nonionic surfactants are selected from Polysorbate 20, Polysorbate-40, Polysorbate-60, and Polysorbate-80. The polyvinyl pyrolidone may preferably be Plasdone C15, a pharmaceutical grade of polyvinyl pyrolidone. Preferred cyclodextrins are hydroxypropyl-β-cyclodextrin, hydroxypropyl-γ-cyclodextrin, and methyl-β-cyclodextrin.

There are multiple acceptable techniques for extraction and isolation of saponins from Quillaja saponaria Molina bark. Acceptable procedures for purifying the saponins of the present invention from Quillaja saponaria Molina bark, measuring the saponins for immune adjuvant activity, and characterizing the substantially pure saponins are disclosed in U.S. Pat. Nos. 5,057,540 and 5,583,112.

Aqueous extracts of Quillaja saponaria bark are also available commercially. These are dark brown, foamy extracts that contain many compounds (tannins, polyphenolics, saponins) that can be analyzed by a method such as reversed phase HPLC.

An example of a reversed phase HPLC analysis of a typical bark extract that is suitable for purification of saponins is shown in FIG. 1 of U.S. Pat. No. 6,231,859, which is incorporated herein in its entirety.

Partial purification to enrich the saponin fraction and to remove the majority of tannins and polyphenolics can be accomplished by dialysis of the extract against water through a 10,000 molecular weight membrane or ultrafiltration. The saponin fraction is retained.

Alternatively, an aqueous saponin extract can be pretreated with polyvinylpolypyrrolidone to remove high molecular weight tannins and polyphenolics through absorption of these compounds.

Residual tannins and polyphenolics can then be removed from the saponin fraction by diafiltration against water. The saponin fraction, which forms micelles, is retained by ultrafiltration membranes of 10,000 to 30,000 molecular weight cutoff pore size. This yields a partially purified extract that consists predominantly of diverse saponins.

Separation of saponins can be accomplished by chromatography in organic solvents or organic solvent/water mixtures. A separation of saponins on silica was described in U.S. Pat. No. 5,057,540. This yields saponins of intermediate purity (enriched in an individual saponin, but less than substantially pure).

Alternatively, other solvent systems on silica gel or the use of reverse phase chromatography can be used to accomplish the initial separation of saponins. This initial purification step can then typically be followed by reversed phase chromatography or similar HPLC step to purify the saponins to near homogeneity.

For example, saponin extract may be recovered from plant cell material freshly extracted from Quillaja trees. Dialyzed extract is then purified on an ion exchange column, e.g., the DE-52 type, followed by Sephadex G50 gel filtration. Ultrafiltration may be used instead of gel filtration. The purified saponin composition is then subjected to RP-HPLC analysis on a VYDAC C4 column, eluted with 30-45% acetonitrile in a 0.15% aqueous TFA-solution.

The substantially pure saponins useful in the present invention may also be isolated from fresh plant material consisting of substantially living cells as disclosed in WO 95/09179, or the previously described procedures.

The same procedure may be performed on plant cell material obtained by means of tissue culture or suspension cell culture. See, e.g., U.S. Pat. No. 5,716,848, which is incorporated herein by reference in its entirety.

General guidance on the use of saponins, Quil-A, and QS-21 can be found in the referenced patents. The amount of saponin present in a pharmaceutically effective composition should contain about 0.1 to 5,000 micrograms or more of a saponin. The amount of saponin present in a pharmaceutically effective composition is more preferably from about 1 to about 1000 micrograms, more preferably from about 5 to 500 micrograms, and most preferably from about 10 to 100 micrograms. In certain specific embodiments, the amount of saponin present in a pharmaceutical composition of the invention is 1, 2, 3, 5, 10, 15, 20, 25, 30, 40, 50, 60, 70, 80, 90, 100, 110, 125 or 150 micrograms.

In certain embodiments of the present invention, a composition of the invention comprises a saponin covalently linked to an antigenic molecule. Covalent linkage of antigens to saponins is fully described in U.S. Pat. No. 5,583,112 at column 9, lines 4-67 and column 10, line 1-37. Additionally, one of skill in the art will be able to establish crosslinking conditions without undue experimentation such that covalent linkage of saponin to antigen and hsp to antigen may be carried out simultaneously.

Formulations of pharmaceutical compositions comprising saponins and procedures for their manufacture can be found in the literature and in the U.S. Patents incorporated by reference into this Description. Saponin formulations referred to are provided herein as nonlimiting examples.

In U.S. Pat. No. 5,583,112 at column 22, lines 11-17, a pharmaceutically effective composition for intradermal administration was made by reconstituting lyophilized “Quil A”, a crude saponin mixture, into a phosphate buffered saline (PBS) solution and mixed with a solution containing 10 micrograms of Bovine Serum Albumin (BSA) to achieve a final volume of 200 microliters for intradermal injection. The effective amount of “Quil A” was found to be approximately 30-77 micrograms of “Quil A” by dry weight.

In U.S. Pat. No. 5,583,112 at column 23, lines 35-38, a pharmaceutically effective composition for subcutaneous administration was made by mixing a stock saline solution of ovalbumin and 10 micrograms of QS-21.

Also in U.S. Pat. No. 5,583,112 at Column 23, lines 35-38, a pharmaceutically effective composition for subcutaneous administration was made by chemically crosslinking QS-21 to lysozyme as described in Example 18 of the patent and resuspending lyophilized QS-21/lysozyme conjugate into 200 microliters of PBS (pH 7) for a final concentration of 10 micrograms of lysozyme and 1.6 micrograms of QS-21.

In a paper by Wu et al. (1994, Cellular Immunology 154:393-406), a pharmaceutically effective composition for subcutaneous or intraperitoneal administration is disclosed containing 25 micrograms ovalbumin absorbed to 250 micrograms of Al(OH)₃ and 20 micrograms of QS-21 per immunization dose.

In another paper by Wu et al. (1994, J. Immun. 148(5):1519-1525), a pharmaceutically effective composition for immunization is disclosed containing 25 micrograms of a truncated recombinant HIV-1 envelope protein absorbed to 250 micrograms of Al(OH)₃ and 10 micrograms of QS-21 in a sterile saline per immunization dose.

As a last non-limiting example, a pharmaceutically effective vaccine has recently been tested in human patients containing 5-500 micrograms of a synthetic nonapeptide and 100 micrograms of QS-21 in 500 microliters PBS (pH 7.4) per intradermally-administered dose (Lewis et al., 2000, Int. J. Cancer 87(3):391-398).

The optimum amount of a specific saponin for use with a specific composition of the invention may vary. Optimization of the specific saponin amount for a given composition is, as demonstrated by the examples cited above, well within the purview of the skilled artisan.

4.4. Determination of Immunogenicity

The compositions of the invention can be assayed for immunogenicity using any method known in the art. In certain embodiments, the antigenicity of the individual components of a composition of the invention can be assayed alone or in combination, and compared to the antigenicity of the composition as whole, to determine the enhancement of the immunogenicity of the composition over the immunogenicity of its individual components. For example, the immunogenicity of an hsp-antigenic molecule complex can be assayed and compared to immunogenicity of a composition comprising the hsp-antigenic molecule and a saponin. Preferably, a composition of the invention is more immunogenic than the immunogenicity of its individual components, and, more preferably is synergistically more immunogenic than the immunogenicity of its individual components. In one specific embodiment, an immunogenic composition of the invention comprises sub-immunogenic amounts of its individual components, i.e., comprises, for example, an hsp-antigenic molecule in an amount that is in itself non-immunogenic, but is immunogenic when combined with a saponin according to the disclosed methods.

By way of example but not limitation, any one of the following procedures can be used. In a preferred embodiment, the ELISPOT assay is used (see, infra, Section 4.4.4).

4.4.1. The MLTC Assay

Briefly, mice are injected with an amount of an α2M or hsp-antigenic molecule complex, using any convenient route of administration. As a negative control, other mice are injected with α2M or hsp-control complexes (e.g. α2M or hsp stripped of antigenic molecules or complexed with non-antigenic molecules). Cells known to contain specific antigens, e.g. tumor cells or cells infected with an agent of an infectious disease, may act as a positive control for the assay. The mice are injected twice, 7-10 days apart. Ten days after the last immunization, the spleens are removed and the lymphocytes released. The released lymphocytes may be re-stimulated subsequently in vitro by the addition of dead cells that expressed the antigen of interest.

For example, 8×10⁶ immune spleen cells may be stimulated with 4×10⁴ mitomycin C treated or γ-irradiated (5-10,000 rads) cells containing the antigen of interest (or cells transfected with an appropriate gene, as the case may be) in 3 ml RPMI medium containing 10% fetal calf serum. In certain cases 33% secondary mixed lymphocyte culture supernatant may be included in the culture medium as a source of T cell growth factors (See, Glasebrook et al., 1980, J. Exp. Med. 151:876). To test the primary cytotoxic T cell response after immunization, spleen cells may be cultured without stimulation. In some experiments spleen cells of the immunized mice may also be re-stimulated with antigenically distinct cells, to determine the specificity of the cytotoxic T cell response.

Six days later the cultures are tested for cytotoxicity in a 4 hour ⁵¹Cr-release assay (See, Palladino et al., 1987, Cancer Res. 47:5074-5079 and Blachere et al., 1993, J. Immunotherapy 14:352-356). In this assay, the mixed lymphocyte culture is added to a target cell suspension to give different effector:target (E:T) ratios (usually 1:1 to 40:1). The target cells are prelabelled by incubating 1×10⁶ target cells in culture medium containing 20 mCi⁵¹Cr/ml for one hour at 37° C. The cells are washed three times following labeling. Each assay point (E:T ratio) is performed in triplicate and the appropriate controls incorporated to measure spontaneous ⁵¹Cr release (no lymphocytes added to assay) and 100% release (cells lysed with detergent). After incubating the cell mixtures for 4 hours, the cells are pelletted by centrifugation at 200 g for 5 minutes. The amount of ⁵¹Cr released into the supernatant is measured by a gamma counter. The percent cytotoxicity is measured as cpm in the test sample minus spontaneously released cpm divided by the total detergent released cpm minus spontaneously released cpm.

In order to block the MHC class I cascade a concentrated hybridoma supernatant derived from K-44 hybridoma cells (an anti-MHC class I hybridoma) is added to the test samples to a final concentration of 12.5%.

4.4.2. CD4+ T Cell Proliferation Assay

Primary T cells are obtained from spleen, fresh blood, or CSF and purified by centrifugation using FICOLL-PAQUE PLUS (Pharmacia, Uppsala, Sweden) essentially as described by Kruse and Sebald, 1992, EMBO J. 11: 3237-3244. The peripheral blood mononuclear cells are incubated for 7-10 days with a lysate of cells expressing an antigenic molecule. Antigen presenting cells may, optionally be added to the culture 24 to 48 hours prior to the assay, in order to process and present the antigen in the lysate. The cells are then harvested by centrifugation, and washed in RPMI 1640 media (GibcoBRL, Gaithersburg, Md.). 5×10⁴ activated T cells/well are in RPMI 1640 media containing 10% fetal bovine serum, 10 mM HEPES, pH 7.5, 2 mM L-glutamine, 100 units/ml penicillin G, and 100 μg/ml streptomycin sulphate in 96 well plates for 72 hrs at 37° C., pulsed with 1 μCi ³H-thymidine (DuPont NEN, Boston, Mass.)/well for 6 hrs, harvested, and radioactivity measured in a TOPCOUNT scintillation counter (Packard Instrument Co., Meridien, Conn.).

4.4.3. Antibody Response Assay

In a certain embodiment of the invention, the immunogenicity of an α2M or hsp-antigenic molecule complex is determined by measuring antibodies produced in response to the vaccination with the complex. In one mode of the embodiment, microtitre plates (96-well Immuno Plate II, Nunc) are coated with 50 μl/well of a 0.75 μg/ml solution of a purified, non-α2M or hsp-complexed form of the antigenic molecule used in the vaccine (e.g. Aβ42) in PBS at 4° C. for 16 hours and at 20° C. for 1 hour. The wells are emptied and blocked with 200 μl PBS-T-BSA (PBS containing 0.05% (v/v) TWEEN 20 and 1% (w/v) bovine serum albumin) per well at 20° C. for 1 hour, then washed 3 times with PBS-T. Fifty μl/well of plasma or CSF from a vaccinated animal (such as a model mouse or a human patient) is applied at 20° C. for 1 hour, and the plates are washed 3 times with PBS-T. The anti-peptide antibody activity is then measured calorimetrically after incubating at 20° C. for 1 hour with 50 μl/well of sheep anti-mouse or anti-human immunoglobulin, as appropriate, conjugated with horseradish peroxidase (Amersham) diluted 1:1,500 in PBS-T-BSA and (after 3 further PBS-T washes as above) with 50 μl of an o-phenylene diamine (OPD)-H₂O₂ substrate solution. The reaction is stopped with 150 μl of 2M H₂SO₄ after 5 minutes and absorbance is determined in a Kontron SLT-210 photometer (SLT Lab-instr., Zurich, Switzerland) at 492 nm (ref. 620 nm).

4.4.4. Cytokine Detection Assays

The CD4+ T cell proliferative response to the compositions of the invention may be measured by detection and quantitation of the levels of specific cytokines. In one embodiment, for example, intracellular cytokines may be measured using an IFN-γ detection assay to test for immunogenicity of a complex of the invention. In an example of this method, peripheral blood mononuclear cells from a subject treated with a composition of the invention are stimulated with peptide antigens of a given tumor or with peptide antigens of an agent of infectious disease. Cells are then stained with T cell-specific labeled antibodies detectable by flow cytometry, for example FITC-conjugated anti-CD8 and PerCP-labeled anti-CD4 antibodies. After washing, cells are fixed, permeabilized, and reacted with dye-labeled antibodies reactive with human IFN-γ (PE-anti-IFN-γ). Samples are analyzed by flow cytometry using standard techniques.

Alternatively, a filter immunoassay, the enzyme-linked immunospot assay (ELISPOT) assay, may be used to detect specific cytokines surrounding a T cell. In one embodiment, for example, a nitrocellulose-backed microtiter plate is coated with a purified cytokine-specific primary antibody, i.e., anti-IFN-γ, and the plate is blocked to avoid background due to nonspecific binding of other proteins. A sample of mononuclear blood cells containing cytokine-secreting cells is obtained from a subject treated with a composition of the invention, which sample is diluted onto the wells of the microtitre plate. A label, e.g., biotin-labeled, secondary anti-cytokine antibody, is added. The antibody-cytokine complex can then be detected, i.e. by enzyme-conjugated streptavidin—cytokine-secreting cells will appear as “spots” by visual, microscopic, or electronic detection methods.

4.4.5. Tetramer Assay

In another embodiment, the “tetramer staining” assay (Altman et al., 1996, Science 274: 94-96) may be used to identify antigen-specific T-cells. For example, in one embodiment, an MHC molecule containing a specific peptide antigen, such as a tumor-specific antigen, is multimerized to make soluble peptide tetramers and labeled, for example, by complexing to streptavidin. The MHC-peptide antigen complex is then mixed with a population of T cells obtained from a subject treated with a composition of the invention. Biotin is then used to stain T cells which express the antigen of interest, i.e., the tumor-specific antigen.

4.5. Recombinant Expression of hsps or α2M

In certain embodiments of the invention, the compositions and methods comprise recombinant hsps, alone or complexed to antigenic molecules, or hsp-antigenic molecule complexes prepared from cells that express enhanced levels of hsps through recombinant means. In other embodiments of the invention, the compositions and methods comprise recombinant α2M or α2M-antigenic molecule complexes comprising recombinant α2M. In this regard, any method known to the skilled artisan may be used for obtaining and manipulating recombinant hsp or α2M sequences. Described below are non-limiting examples of such methods.

4.5.1. hsp Sequences

Amino acid sequences and nucleotide sequences of many hsps are generally available in sequence databases, such as GenBank. Computer programs, such as Entrez, can be used to browse the database, and retrieve any amino acid sequence and genetic sequence data of interest by accession number. These databases can also be searched to identify sequences with various degrees of similarities to a query sequence using programs, such as FASTA and BLAST, which rank the similar sequences by alignment scores and statistics. Such nucleotide sequences of non-limiting examples of hsps that can be used for the compositions, methods, and for preparation of the hsp-antigenic molecule complexes of the invention are as follows: human hsp70, Genbank Accession No.M24743, Hunt et al., 1995, Proc. Natl. Acad. Sci. U.S.A., 82: 6455-6489; human hsp90, Genbank Accession No.X15183, Yamazaki et al., Nucl. Acids Res. 17: 7108; human gp96, Genbank Accession No.X15187, Maki et al., 1990, Proc. Natl. Acad. Sci. U.S.A. 87: 5658-5562; human BiP, Genbank Accession No.M19645, Ting et al., 1988, DNA 7: 275-286; human hsp27, Genbank Accession No.M24743, Hickey et al., 1986, Nucleic Acids Res. 14: 4127-45; mouse hsp70, Genbank Accession No.M35021, Hunt et al., 1990, Gene 87: 199-204; mouse gp96, Genbank Accession No.M16370, Srivastava et al., 1987, Proc. Natl. Acad. Sci. U.S.A. 85: 3807-3811; and mouse BiP, Genbank Accession No.U16277, Haas et al., 1988, Proc. Natl. Acad. Sci. U.S.A. 85: 2250-2254. Due to the degeneracy of the genetic code, the term “hsp gene”, as used herein, refers not only to the naturally occurring nucleotide sequence but also encompasses all the other degenerate DNA sequences that encode the hsp.

Once the nucleotide sequence encoding the hsp of choice has been identified, the nucleotide sequence, or a fragment thereof, can be obtained (e.g. from commercial sources or by PCR as described below) and cloned into an expression vector for recombinant expression. The expression vector can then be introduced into a host cell for propogation of the hsp. Methods for recombinant production of hsps are quite well known, as exemplified herein.

The DNA may be obtained by DNA amplification or molecular cloning directly from a tissue, cell culture, or cloned DNA (e.g., a DNA “library”) using standard molecular biology techniques (see e.g., Methods in Enzymology, 1987, volume 154, Academic Press; Sambrook et al., 1989, Molecular Cloning—A Laboratory Manual, 2nd Edition, Cold Spring Harbor Press, New York; and Current Protocols in Molecular Biology, Ausubel et al. (eds.), Greene Publishing Associates and Wiley Interscience, New York, each of which is incorporated herein by reference in its entirety). Clones derived from genomic DNA may contain regulatory and intron DNA regions in addition to coding regions; clones derived from cDNA will contain only exon sequences. Whatever the source, the hsp gene should be cloned into a suitable vector for propagation of the gene.

In a preferred embodiment, DNA can be amplified from genomic or cDNA by polymerase chain reaction (PCR) amplification using primers designed from the known sequence of a related or homologous hsp. PCR is used to amplify the desired sequence in DNA clone or a genomic or cDNA library, prior to selection. PCR can be carried out, e.g., by use of a thermal cycler and Taq polymerase (Gene Amp®). The polymerase chain reaction (PCR) is commonly used for obtaining genes or gene fragments of interest. For example, a nucleotide sequence encoding an hsp of any desired length can be generated using PCR primers that flank the nucleotide sequence encoding open reading frame. Alternatively, an hsp gene sequence can be cleaved at appropriate sites with restriction endonuclease(s) if such sites are available, releasing a fragment of DNA encoding the hsp gene. If convenient restriction sites are not available, they may be created in the appropriate positions by site-directed mutagenesis and/or DNA amplification methods known in the art (see, for example, Shankarappa et al., 1992, PCR Method Appl. 1: 277-278). The DNA fragment that encodes the hsp is then isolated, and ligated into an appropriate expression vector, care being taken to ensure that the proper translation reading frame is maintained.

In an alternative embodiment, for the molecular cloning of an hsp gene from genomic DNA, DNA fragments are generated to form a genomic library. Since some of the sequences encoding related hsps are available and can be purified and labeled, the cloned DNA fragments in the genomic DNA library may be screened by nucleic acid hybridization to a labeled probe (Benton and Davis, 1977, Science 196: 180; Grunstein and Hogness, 1975, Proc. Natl. Acad. Sci. U.S.A. 72: 3961). Those DNA fragments with substantial homology to the probe will hybridize. It is also possible to identify an appropriate fragment by restriction enzyme digestion(s) and comparison of fragment sizes with those expected according to a known restriction map.

Alternatives to isolating the hsp genomic DNA include, but are not limited to, chemically synthesizing the gene sequence itself from a known sequence or synthesizing a cDNA to the mRNA which encodes the hsp. For example, RNA for cDNA cloning of the hsp gene can be isolated from cells which express the hsp. A cDNA library may be generated by methods known in the art and screened by methods, such as those disclosed for screening a genomic DNA library. If an antibody to the hsp is available, the hsp may be identified by binding of a labeled antibody to the hsp-synthesizing clones.

Other specific embodiments for the cloning of a nucleotide sequence encoding an hsp, are presented as examples but not by way of limitation, as follows: In a specific embodiment, nucleotide sequences encoding an hsp can be identified and obtained by hybridization with a probe comprising a nucleotide sequence encoding hsp under conditions of low to medium stringency. By way of example and not limitation, procedures using such conditions of low stringency are as follows (see also Shilo and Weinberg, 1981, Proc. Natl. Acad. Sci. U.S.A. 78: 6789-6792). Filters containing DNA are pretreated for 6 h at 40° C. in a solution containing 35% formamide, 5×SSC, 50 mM Tris-HCl (pH7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 40° C., and then washed for 1.5 h at 55° C. in a solution containing 2×SSC, 25 mM Tris-HCl (pH7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and reexposed to film. Other conditions of low stringency which may be used are well known in the art (e.g., as employed for cross-species hybridizations).

Any technique for mutagenesis known in the art can be used to modify individual nucleotides in a DNA sequence, for purpose of making amino acid substitution(s) in the expressed peptide sequence, or for creating/deleting restriction sites to facilitate further manipulations. Such techniques include but are not limited to, chemical mutagenesis, in vitro site-directed mutagenesis (Hutchinson et al., 1978, J. Biol. Chem. 253: 6551), oligonucleotide-directed mutagenesis (Smith, 1985, Ann. Rev. Genet. 19: 423-463; Hill et al., 1987, Methods Enzymol. 155: 558-568), PCR-based overlap extension (Ho et al., 1989, Gene 77: 51-59), PCR-based megaprimer mutagenesis (Sarkar et al., 1990, Biotechniques 8: 404-407), etc. Modifications can be confirmed, e.g., by double-stranded dideoxynucleotide DNA sequencing.

In certain embodiments, a nucleic acid encoding a secretory form of the hsp of choice is used to prepare the compositions and/or practice the methods of the present invention. Such a nucleic acid can be constructed by, e.g., deleting the coding sequence for an ER retention signal, KDEL. Optionally, the KDEL coding sequence is replaced with a molecular tag, such as the Fc portion of murine IgG1, to facilitate the recognition and purification of the hsp. U.S. application Ser. No. 09/253,439, incorporated herein by reference, demonstrates that deletion of the ER retention signal of gp96 results in the secretion of gp96-Ig peptide-complexes from transfected tumor cells, and that fusion of the KDEL-deleted gp96 with murine IgG1 facilitated its detection by ELISA and FACS analysis, and its purification by affinity chromatography with the aid of Protein A.

4.5.2. α2M Sequences

α2M polypeptides may be produced by recombinant DNA techniques, synthetic methods, or by enzymatic or chemical cleavage of native α2M polypeptides. Described below are methods for producing such α2M polypeptides.

In various aspects, the invention relates to compositions comprising amino acid sequences of α2M, and fragments, derivatives, analogs, and variants thereof. Nucleic acids encoding α2M are provided, as well as nucleic acids complementary to and capable of hybridizing to such nucleic acids.

Any eukaryotic cell may serve as the nucleic acid source for obtaining the coding region of an α2M gene. Nucleic acid sequences encoding α2M can be isolated from vertebrate, mammalian, as well as primate sources, including humans.

Amino acid sequences and nucleotide sequences of naturally occurring α2M polypeptides are generally available in sequence databases, such as GenBank. Non-limiting examples of α2M sequences that can be used for preparation of the α2M polypeptides of the invention are as follows: Genbank Accession Nos. M11313, P01023, AAA51551; see Kan et al., 1985, Proc. Nat. Acad. Sci. U.S.A. 82: 2282-2286. Due to the degeneracy of the genetic code, the term “α2M gene”, as used herein, refers not only to the naturally occurring nucleotide sequence but also encompasses all the other degenerate DNA sequences that encode an α2M polypeptide. Computer programs, such as Entrez, can be used to browse the database, and retrieve any amino acid sequence and genetic sequence data of interest by accession number. These databases can also be searched to identify sequences with various degrees of similarities to a query sequence using programs, such as FASTA and BLAST, which rank the similar sequences by alignment scores and statistics. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al., 1997, Nucleic Acids Res.25:3389-3402. Alternatively, PSI-Blast can be used to perform an iterated search which detects distant relationships between molecules (Altschul et al., 1997, supra). When utilizing BLAST, Gapped BLAST, and PSI-Blast programs, the default parameters of the respective programs (e.g., XBLAST and NBLAST) can be used (see http://www.ncbi.nlm.nih.gov).

The DNA may be obtained by standard procedures known in the art by DNA amplification or molecular cloning directly from a tissue, cell culture, or cloned DNA (e.g., a DNA “library”). Clones derived from genomic DNA may contain regulatory and intron DNA regions in addition to coding regions; clones derived from cDNA will contain only exon sequences. Whatever the source, the α2M gene should be cloned into a suitable vector for propagation of the gene.

In a preferred embodiment, DNA can be amplified from genomic or cDNA by polymerase chain reaction (PCR) amplification using primers designed from the known sequence of a related or homologous α2M. PCR is used to amplify the desired sequence in DNA clone or a genomic or cDNA library, prior to selection. PCR can be carried out, e.g., by use of a thermal cycler and Taq polymerase (sold under the trademark GENE AMP). The DNA being amplified can include cDNA or genomic DNA from any species. Oligonucleotide primers representing known nucleic acid sequences of related HSPs can be used as primers in PCR. In a preferred aspect, the oligonucleotide primers represent at least part of the α2M gene that is highly conserved between α2M genes of different species. One can choose to synthesize several different degenerate primers, for use in the PCR reactions. It is also possible to vary the stringency of hybridization conditions used in priming the PCR reactions, to allow for greater or lesser degrees of nucleotide sequence similarity between the known α2M nucleotide sequence and the nucleic acid homolog being isolated. For cross species hybridization, low stringency conditions are preferred. For same species hybridization, moderately stringent conditions are preferred. After successful amplification, the sequence encoding an α2M may be cloned and sequenced. If the size of the coding region of the α2M gene being amplified is too large to be amplified in a single PCR, several PCR covering the entire gene, preferably with overlapping regions, may be carried out, and the products of the PCR ligated together to form the entire coding sequence. Alternatively, if a segment of an α2M gene is amplified, that segment may be cloned, and utilized as a probe to isolate a complete cDNA or genomic clone.

In another embodiment, for the molecular cloning of an α2M gene from genomic DNA, DNA fragments are generated to form a genomic library. Since some of the sequences encoding related α2Ms are available and can be purified and labeled, the cloned DNA fragments in the genomic DNA library may be screened by nucleic acid hybridization to the labeled probe (Benton and Davis, 1977, Science 196:180; Grunstein and Hogness, 1975, Proc. Natl. Acad. Sci. U.S.A. 72:3961). Those DNA fragments with substantial homology to the probe will hybridize. It is also possible to identify the appropriate fragment by restriction enzyme digestion(s) and comparison of fragment sizes with those expected according to a known restriction map if such is available.

Alternatives to isolating the α2M genomic DNA include, but are not limited to, chemically synthesizing the gene sequence itself from a known sequence or making cDNA to the mRNA which encodes α2M. For example, RNA for cDNA cloning of the α2M gene can be isolated from cells which express α2M. A cDNA library may be generated by methods known in the art and screened by methods, such as those disclosed for screening a genomic DNA library. If an antibody to α2M is available, α2M may be identified by binding of labeled antibody to the putatively α2M synthesizing clones.

Other specific embodiments for the cloning of a nucleotide sequence encoding an α2M, are presented as examples but not by way of limitation, as follows:

In a specific embodiment, nucleotide sequences encoding α2M proteins within a family can be identified and obtained by hybridization with a probe comprising nucleotide sequence encoding α2M under conditions of low to medium stringency.

By way of example and not limitation, procedures using such conditions of low stringency are as follows (see also Shilo and Weinberg, 1981, Proc. Natl. Acad. Sci. USA 78:6789-6792). Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and reexposed to film. Other conditions of low stringency which may be used are well known in the art (e.g., as employed for cross-species hybridizations).

An α2M gene fragment can be inserted into an appropriate cloning vector and introduced into host cells so that many copies of the gene sequence are generated. A large number of vector-host systems known in the art may be used such as, but not limited to, bacteriophages such as lambda derivatives, or plasmids such as pBR322 or pUC plasmid derivatives or the Bluescript vector (Stratagene).

Any technique for mutagenesis known in the art can be used to modify individual nucleotides in a DNA sequence, for purpose of making amino acid substitution(s) in the expressed peptide sequence, or for creating/deleting restriction sites to facilitate further manipulations. Such techniques include but are not limited to, chemical mutagenesis, in vitro site-directed mutagenesis (Hutchinson et al., 1978, J. Biol. Chem 253:6551), oligonucleotide-directed mutagenesis (Smith, 1985, Ann. Rev. Genet. 19:423-463; Hill et al., 1987, Methods Enzymol. 155:558-568), PCR-based overlap extension (Ho et al., 1989, Gene 77:51-59), PCR-based megaprimer mutagenesis (Sarkar et al., 1990, Biotechniques, 8:404-407), etc. Modifications can be confirmed by double stranded dideoxy DNA sequencing.

The polymerase chain reaction (PCR) is commonly used for obtaining genes or gene fragments of interest. For example, a nucleotide sequence encoding α2M polypeptide of any desired length can be generated using PCR primers that flank the nucleotide sequence encoding α2M, or the peptide-binding domain thereof. Alternatively, an α2M gene sequence can be cleaved at appropriate sites with restriction endonuclease(s) if such sites are available, releasing a fragment of DNA encoding α2M, or the peptidebinding domain thereof. If convenient restriction sites are not available, they may be created in the appropriate positions by site-directed mutagenesis and/or DNA amplification methods known in the art (see, for example, Shankarappa et al., 1992, PCR Method Appl. 1:277-278). The DNA fragment that encodes α2M, or the peptide-binding domain thereof, is then isolated, and ligated into an appropriate expression vector, care being taken to ensure that the proper translation reading frame is maintained.

Alpha (2) macroglobulin polypeptides may be expressed as fusion proteins to facilitate recovery and purification from the cells in which they are expressed. For example, an α2M polypeptide may contain a signal sequence leader peptide to direct its translocation across the ER membrane for secretion into culture medium. Further, an α2M polypeptide may contain an affinity label, such as a affinity label, fused to any portion of the α2M polypeptide not involved in binding antigenic peptide, such as for example, the carboxyl terminal. The affinity label can be used to facilitate purification of the protein, by binding to an affinity partner molecule.

Various methods for production of such fusion proteins are well known in the art. The manipulations which result in their production can occur at the gene or protein level, preferably at the gene level. For example, the cloned coding region of an α2M polypeptide may be modified by any of numerous recombinant DNA methods known in the art (Sambrook et al., 1990, Molecular Cloning, A Laboratory Manual, 2d ed., Cold Spring Harbor Laboratory, Cold Spring Harbor, N.Y.; Ausubel et al., in Chapter 8 of Current Protocols in Molecular Biology, Greene Publishing Associates and Wiley Interscience, New York). It will be apparent from the following discussion that substitutions, deletions, insertions, or any combination thereof are introduced or combined to arrive at a final nucleotide sequence encoding an α2M polypeptide.

In various embodiments, fusion proteins comprising the α2M polypeptide may be made using recombinant DNA techniques. For example, a recombinant gene encoding an α2M polypeptide may be constructed by introducing an α2M gene fragment in the proper reading frame into a vector containing the sequence of an affinity label, such that the α2M polypeptide is expressed as a peptide-tagged fusion protein. Affinity labels, which may be recognized by specific binding partners, may be used for affinity purification of the α2M polypeptide.

In a preferred embodiment, the affinity label is fused at its amino terminal to the carboxyl terminal of α2M. The precise site at which the fusion is made in the carboxyl terminal is not critical. The optimal site can be determined by routine experimentation.

A variety of affinity labels known in the art may be used, such as, but not limited to, the immunoglobulin constant regions, polyhistidine sequence (Petty, 1996, Metal-chelate affinity chromatography, in Current Protocols in Molecular Biology, Vol. 2, Ed. Ausubel et al., Greene Publish. Assoc. & Wiley Interscience), glutathione S-transferase (GST; Smith, 1993, Methods Mol. Cell Bio. 4:220-229), the E. coli maltose binding protein (Guan et al., 1987, Gene 67:21-30), and various cellulose binding domains (U.S. Pat. Nos. 5,496,934; 5,202,247; 5,137,819; Tomme et al., 1994, Protein Eng. 7:117-123), etc. Other affinity labels may impart fluorescent properties to an α2M polypeptide, e.g., portions of green fluorescent protein and the like. Other possible affinity labels are short amino acid sequences to which monoclonal antibodies are available, such as but not limited to the following well known examples, the FLAG epitope, the myc epitope at amino acids 408-439, the influenza virus hemagglutinin (HA) epitope. Other affinity labels are recognized by specific binding partners and thus facilitate isolation by affinity binding to the binding partner which can be immobilized onto a solid support. Some affinity labels may afford the α2M polypeptide novel structural properties, such as the ability to form multimers. Dimerization of an α2M polypeptide with a bound peptide may increase avidity of interaction between the α2M polypeptide and its partner in the course of antigen presentation. These affinity labels are usually derived from proteins that normally exist as homopolymers. Affinity labels such as the extracellular domains of CD8 (Shiue et al., 1988, J. Exp. Med. 168:1993-2005), or CD28 (Lee et al., 1990, J. Immunol. 145:344-352), or portions of the immunoglobulin molecule containing sites for interchain disulfide bonds, could lead to the formation of multimers. As will be appreciated by those skilled in the art, many methods can be used to obtain the coding region of the above-mentioned affinity labels, including but not limited to, DNA cloning, DNA amplification, and synthetic methods. Some of the affinity labels and reagents for their detection and isolation are available commercially.

A preferred affinity label is a non-variable portion of the immunoglobulin molecule. Typically, such portions comprise at least a functionally operative CH2 and CH3 domain of the constant region of an immunoglobulin heavy chain. Fusions are also made using the carboxyl terminus of the Fc portion of a constant domain, or a region immediately amino-terminal to the CH1 of the heavy or light chain. Suitable immunoglobulin-based affinity label may be obtained from IgG-1, -2, -3, or -4 subtypes, IgA, IgE, IgD, or IgM, but preferably IgG2. Preferably, a human immunoglobulin is used when the α2M polypeptide is intended for in vivo use for humans. Many DNA encoding immunoglobulin light or heavy chain constant regions is known or readily available from cDNA libraries. See, for example, Adams et al., Biochemistry, 1980, 19:2711-2719; Gough et al., 1980, Biochemistry, 19:2702-2710; Dolby et al., 1980, Proc. Natl. Acad. Sci. U.S.A., 77:6027-6031; Rice et al., 1982, Proc. Natl. Acad. Sci. U.S.A., 79:7862-7865; Falkner et al., 1982, Nature, 298:286-288; and Morrison et al., 1984, Ann. Rev. Immunol, 2:239-256. Because many immunological reagents and labeling systems are available for the detection of immunoglobulins, the α2M polypeptide-Ig fusion protein can readily be detected and quantified by a variety of immunological techniques known in the art, such as the use of enzyme-linked immunosorbent assay (ELISA), immunoprecipitation, fluorescence activated cell sorting (FACS), etc. Similarly, if the affinity label is an epitope with readily available antibodies, such reagents can be used with the techniques mentioned above to detect, quantitate, and isolate the α2M polypeptide containing the affinity label. In many instances, there is no need to develop specific antibodies to the α2M polypeptide.

A particularly preferred embodiment is a fusion of an α2M polypeptide to the hinge, the CH2 and CH3 domains of human immunoglobulin G-1 (IgG-1; see Bowen et al,1996, J. Immunol. 156:442-49). This hinge region contains three cysteine residues which are normally involved in disulfide bonding with other cysteines in the Ig molecule. Since none of the cysteines are required for the peptide to function as a tag, one or more of these cysteine residues may optionally be substituted by another amino acid residue, such as for example, serine.

Various leader sequences known in the art can be used for the efficient secretion of α2M polypeptide from bacterial and mammalian cells (von Heijne, 1985, J. Mol. Biol. 184:99-105). Leader peptides are selected based on the intended host cell, and may include bacterial, yeast, viral, animal, and mammalian sequences. For example, the herpes virus glycoprotein D leader peptide is suitable for use in a variety of mammalian cells. A preferred leader peptide for use in mammalian cells can be obtained from the V-J2-C region of the mouse immunoglobulin kappa chain (Bernard et al., 1981, Proc. Natl. Acad. Sci. 78:5812-5816). Preferred leader sequences for targeting α2M polypeptide expression in bacterial cells include, but are not limited to, the leader sequences of the E. coli proteins OmpA (Hobom et al., 1995, Dev. Biol. Stand. 84:255-262), Pho A (Oka et al., 1985, Proc. Natl. Acad. Sci 82:7212-16), OmpT (Johnson et al., 1996, Protein Expression 7:104-113), LamB and OmpF (Hoffinan & Wright, 1985, Proc. Natl. Acad. Sci. USA 82:5107-5111), β-lactamase (Kadonaga et al., 1984, J. Biol. Chem. 259:2149-54), enterotoxins (Morioka-Fujimoto et al., 1991, J. Biol. Chem. 266:1728-32), and the Staphylococcus aureus protein A (Abrahmsen et al., 1986, Nucleic Acids Res. 14:7487-7500), and the B. subtilis endoglucanase (Lo et al., Appl. Environ. Microbiol. 54:2287-2292), as well as artificial and synthetic signal sequences (MacIntyre et al., 1990, Mol. Gen. Genet. 221:466-74; Kaiser et al., 1987, Science, 235:312-317).

DNA sequences encoding a desired affinity label or leader peptide, which may be readily obtained from libraries, produced synthetically, or may be available from commercial suppliers, are suitable for the practice of this invention. Such methods are well known in the art.

4.5.3. Expression Systems

Nucleotide sequences encoding an hsp or α2M and/or an antigenic molecule or an hsp-antigenic molecule or α2M-antigenic molecule fusion can be inserted into an expression vector to produce an expression construct for propagation and expression in recombinant cells. An expression construct, as used herein, refers to a nucleotide sequence encoding an hsp, α2M, and/or antigenic molecule operably associated with one or more regulatory regions which allows expression of the hsp, α2M and/or antigenic molecule in an appropriate host cell. “Operably-associated” refers to an association in which the regulatory regions and the hsp, α2M and/or antigenic molecule polypeptide sequence to be expressed are joined and positioned in such a way as to permit transcription, and ultimately, translation of the hsp, α2M and/or antigenic molecule sequence. A variety of expression vectors may be used for the expression of hsps, α2M and/or antigenic molecules, including, but not limited to, plasmids, cosmids, phage, phagemids, or modified viruses. Examples include bacteriophages such as lambda derivatives, or plasmids such as pBR322 or pUC plasmid derivatives or the Bluescript vector (Stratagene). Typically, such expression vectors comprise a functional origin of replication for propagation of the vector in an appropriate host cell, one or more restriction endonuclease sites for insertion of the hsp gene sequence or sequence encoding an antigenic molecule, and one or more selection markers.

For expression of hsps, α2M and/or antigenic molecules in mammalian host cells, a variety of regulatory regions can be used, for example, the SV40 early and late promoters, the cytomegalovirus (CMV) immediate early promoter, and the Rous sarcoma virus long terminal repeat (RSV-LTR) promoter. Inducible promoters that may be useful in mammalian cells include but are not limited to those associated with the metallothionein II gene, mouse mammary tumor virus glucocorticoid responsive long terminal repeats (MMTV-LTR), the β-interferon gene, and the hsp70 gene (Williams et al., 1989, Cancer Res. 49: 2735-42; Taylor et al., 1990, Mol. Cell. Biol. 10: 165-75).

The following animal regulatory regions, which exhibit tissue specificity and have been utilized in transgenic animals, can also be used for the recombinant expression of hsps and/or antigenic molecules in cells of a particular tissue type: elastase I gene control region which is active in pancreatic acinar cells (Swift et al., 1984, Cell 38: 639-646; Omitz et al., 1986, Cold Spring Harbor Symp. Quant. Biol. 50: 399-409; MacDonald, 1987, Hepatology 7: 425-515); insulin gene control region which is active in pancreatic beta cells (Hanahan, 1985, Nature 315: 115-122), immunoglobulin gene control region which is active in lymphoid cells (Grosschedl et al., 1984, Cell 38: 647-658; Adames et al., 1985, Nature 318: 533-538; Alexander et al., 1987, Mol. Cell. Biol. 7: 1436-1444), mouse mammary tumor virus control region which is active in testicular, breast, lymphoid and mast cells (Leder et al., 1986, Cell 45: 485-495), albumin gene control region which is active in liver (Pinkert et al., 1987, Genes Dev. 1: 268-276), alpha-fetoprotein gene control region which is active in liver (Krumlauf et al., 1985, Mol. Cell. Biol. 5: 1639-1648; Hammer et al., 1987, Science 235: 53-58; alpha 1-antitrypsin gene control region which is active in the liver (Kelsey et al., 1987, Genes Dev. 1: 161-171), beta-globin gene control region which is active in myeloid cells (Mogram et al., 1985, Nature 315: 338-340; Kollias et al., 1986, Cell 46: 89-94; myelin basic protein gene control region which is active in oligodendrocyte cells in the brain (Readhead et al., 1987, Cell 48: 703-712); myosin light chain-2 gene control region which is active in skeletal muscle (Sani, 1985, Nature 314: 283-286), and gonadotropic releasing hormone gene control region which is active in the hypothalamus (Mason et al., 1986, Science 234: 1372-1378).

The efficiency of expression of the hsp, α2M or antigenic molecule in a host cell may be enhanced by the inclusion of appropriate transcription enhancer elements in the expression vector, such as those found in SV40 virus, Hepatitis B virus, cytomegalovirus, immunoglobulin genes, metallothionein, β-actin (see Bittner et al., 1987, Methods in Enzymol. 153: 516-544; Gorman, 1990, Curr. Op. in Biotechnol. 1: 36-47).

The expression vector may also contain sequences that permit maintenance and replication of the vector in more than one type of host cell, or integration of the vector into the host chromosome. Such sequences may include but are not limited to replication origins, autonomously replicating sequences (ARS), centromere DNA, and telomere DNA. It may also be advantageous to use shuttle vectors that can be replicated and maintained in at least two types of host cells.

In addition, the expression vector may contain selectable or screenable marker genes for initially isolating or identifying host cells that contain DNA encoding an hsp, α2M and/or antigenic molecule. For long term, high yield production of hsps, α2M and/or antigenic molecules, stable expression in mammalian cells is preferred. A number of selection systems may be used for mammalian cells, including, but not limited, to the Herpes simplex virus thymidine kinase (Wigler et al., 1977, Cell 11: 223), hypoxanthine-guanine phosphoribosyltransferase (Szybalski and Szybalski, 1962, Proc. Natl. Acad. Sci. U.S.A. 48: 2026), and adenine phosphoribosyltransferase (Lowy et al., 1980, Cell 22: 817) genes can be employed in tk−, hgprt− or aprt− cells, respectively. Also, antimetabolite resistance can be used as the basis of selection for dihydrofolate reductase (dhfr), which confers resistance to methotrexate (Wigler et al., 1980, Natl. Acad. Sci. U.S.A. 77: 3567; O'Hare et al., 1981, Proc. Natl. Acad. Sci. U.S.A. 78: 1527); gpt, which confers resistance to mycophenolic acid (Mulligan and Berg, 1981, Proc. Natl. Acad. Sci. U.S.A. 78: 2072); neomycin phosphotransferase (neo), which confers resistance to the aminoglycoside G-418 (Colberre-Garapin et al., 1981, J. Mol. Biol. 150: 1); and hygromycin phosphotransferase (hyg), which confers resistance to hygromycin (Santerre et al., 1984, Gene 30: 147). Other selectable markers, such as but not limited to histidinol and Zeocin™ can also be used.

In order to insert the hsp or α2M coding sequence or the coding sequence of an antigenic molecule into the cloning site of a vector, DNA sequences with regulatory functions, such as promoters, must be attached to the respective coding sequences. To do this, linkers or adapters providing the appropriate compatible restriction sites may be ligated to the ends of cDNA or synthetic DNA encoding an hsp, α2M or antigenic molecule, by techniques well known in the art (Wu et al., 1987, Methods Enzymol. 152: 343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA by use of PCR with primers containing the desired restriction enzyme site.

The expression construct comprising an hsp, α2M, and/or antigenic molecule-coding sequence operably associated with regulatory regions can be directly introduced into appropriate host cells for expression and production of the hsp, α2M, and/or antigenic molecule complexes of the invention without further cloning (see e.g., U.S. Pat. No. 5,580,859). The expression constructs may also contain DNA sequences that facilitate integration of the coding sequence into the genome of the host cell, e.g., via homologous recombination. In this instance, it is not necessary to employ an expression vector comprising a replication origin suitable for appropriate host cells in order to propagate and express the hsp, α2M and/or antigenic molecule in the host cells.

Expression constructs containing cloned hsp or α2M coding sequences or coding sequences for antigenic molecules can be introduced into the mammalian host cell by a variety of techniques known in the art, including but not limited to calcium phosphate mediated transfection (Wigler et al., 1977, Cell 11: 223-232), liposome-mediated transfection (Schaefer-Ridder et al., 1982, Science 215: 166-168), electroporation (Wolff et al., 1987, Proc. Natl. Acad. Sci. 84: 3344), and microinjection (Cappechi, 1980, Cell 22: 479-488).

For long-term, high-yield production of properly processed hsp-peptide complexes, stable expression in mammalian cells is preferred. Cell lines that stably express hsps or α2M and antigenic molecules to produce hsp-peptide complexes for incorporating into the compositions of the present invention may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while the hsp or α2M and antigenic molecule is expressed continuously.

Any of the cloning and expression vectors described herein may be synthesized and assembled from known DNA sequences by techniques well known in the art. The regulatory regions and enhancer elements can be of a variety of origins, both natural and synthetic. Some vectors and host cells may be obtained commercially. Non-limiting examples of useful vectors are described in Appendix 5 of Current Protocols in Molecular Biology, 1988, ed. Ausubel et al., Greene Publish. Assoc. & Wiley Interscience, which is incorporated herein by reference; and the catalogs of commercial suppliers such as Clontech Laboratories, Stratagene Inc., and Invitrogen, Inc.

Alternatively, number of viral-based expression systems may also be utilized with mammalian cells for recombinant expression of hsps, α2M and/or antigenic molecules. Vectors using DNA virus backbones have been derived from simian virus 40 (SV40) (Hamer et al., 1979, Cell 17: 725), adenovirus (Van Doren et al., 1984, Mol. Cell Biol. 4: 1653), adeno-associated virus (McLaughlin et al., 1988, J. Virol. 62: 1963), and bovine papillomas virus (Zinn et al., 1982, Proc. Natl. Acad. Sci. 79: 4897). In cases where an adenovirus is used as an expression vector, the donor DNA sequence may be ligated to an adenovirus transcription/translation control region, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing heterologous products in infected hosts (see, e.g., Logan and Shenk, 1984, Proc. Natl. Acad. Sci. U.S.A. 81: 3655-3659).

Bovine papillomavirus (BPV) can infect many higher vertebrates, including man, and its DNA replicates as an episome. A number of shuttle vectors have been developed for recombinant gene expression which exist as stable, multicopy (20-300 copies/cell) extrachromosomal elements in mammalian cells. Typically, these vectors contain a segment of BPV DNA (the entire genome or a 69% transforming fragment), a promoter with a broad host range, a polyadenylation signal, splice signals, a selectable marker, and “poisonless” plasmid sequences that allow the vector to be propagated in E. coli. Following construction and amplification in bacteria, the expression gene construct is transfected into cultured mammalian cells, for example, by the techniques of calcium phosphate coprecipitation or electroporation. For those host cells that do not manifest a transformed phenotype, selection of transformants is achieved by use of a dominant selectable marker, such as histidinol and G418 resistance. For example, BPV vectors such as pBCMGSNeo and pBCMGHis may be used to express hsps, α2M and/or antigenic molecules (Karasuyama et al., Eur. J. Immunol. 18: 97-104; Ohe et al., Human Gene Therapy 6: 325-33) which may then be transfected into a diverse range of cell types for hsp, α2M or antigenic molecule expression.

Alternatively, the vaccinia 7.5K promoter may be used (see, e.g., Mackett et al., 1982, Proc. Natl. Acad. Sci. U.S.A. 79: 7415-7419; Mackett et al., 1984, J. Virol. 49: 857-864; Panicali et al., 1982, Proc. Natl. Acad. Sci. U.S.A. 79: 4927-4931) In cases where a human host cell is used, vectors based on the Epstein-Barr virus (EBV) origin (OriP) and EBV nuclear antigen 1 (EBNA-1; a trans-acting replication factor) may be used. Such vectors can be used with a broad range of human host cells, e.g., EBO-pCD (Spickofsky et al., 1990, DNA Prot. Eng. Tech. 2: 14-18), pDR2 and λDR2 (available from Clontech Laboratories).

Recombinant hsp, α2M and/or antigenic molecule expression can also be achieved by a retrovirus-based expression system. In contrast to transfection, retroviruses can efficiently infect and transfer genes to a wide range of cell types including, for example, primary hematopoietic cells. In retroviruses such as Moloney murine leukemia virus, most of the viral gene sequences can be removed and replaced with an hsp, α2M and/or antigenic molecule coding sequence or a sequence encoding an antigenic molecule, while the missing viral functions can be supplied in trans. The host range for infection by a retroviral vector can also be manipulated by the choice of envelope used for vector packaging.

For example, a retroviral vector can comprise a 5′ long terminal repeat (LTR), a 3′ LTR, a packaging signal, a bacterial origin of replication, and a selectable marker. The ND-associated antigenic peptide DNA is inserted into a position between the 5′ LTR and 3′ LTR, such that transcription from the 5′ LTR promoter transcribes the cloned DNA. The 5′ LTR comprises a promoter, including but not limited to an LTR promoter, an R region, a U5 region and a primer binding site, in that order. Nucleotide sequences of these LTR elements are well known in the art. A heterologous promoter as well as multiple drug selection markers may also be included in the expression vector to facilitate selection of infected cells (see McLauchlin et al., 1990, Prog. Nucleic Acid Res. and Molec. Biol. 38: 91-135; Morgenstern et al., 1990, Nucleic Acid Res. 18: 3587-3596; Choulika et al., 1996, J. Virol 70: 1792-1798; Boesen et al., 1994, Biotherapy 6: 291-302; Salmons and Gunzberg, 1993, Human Gene Therapy 4: 129-141; and Grossman and Wilson, 1993, Curr. Opin. in Genetics and Devel. 3: 110-114).

The recombinant cells may be cultured under standard conditions of temperature, incubation time, optical density, and media composition. Alternatively, a cell may be cultured under conditions emulating the nutritional and physiological requirements of a cell in which the hsp, α2M or antigenic molecule is endogenously expressed. Modified culture conditions and media may be used to enhance production of hsp-antigenic molecule or α2M-antigenic molecule complexes. For example, recombinant cells may be grown under conditions that promote inducible hsp expression. Any technique known in the art may be applied to establish the optimal conditions for producing hsp-antigenic molecule or α2M-antigenic molecule complexes.

4.6. Therapeutic and Prophylactic Applications of the Compositions of the Invention

In accordance with the present invention, a composition of the invention, comprising an hsp or α2M, optionally an antigenic molecule, and a saponin, is administered to a human subject with cancer, an infectious disease, or an autoimmune disease. In one embodiment, “treatment” or “treating” refers to an amelioration of cancer, an infectious disease, a neurodegenerative or amyloid disease or an autoimmune disease, or at least one discernible symptom thereof. In another embodiment, “treatment” or “treating” refers to an amelioration of at least one measurable physical parameter associated with cancer, an infectious disease, a neurodegenerative or amyloid disease or an autoimmune disease, not necessarily discernible by the subject. In yet another embodiment, “treatment” or “treating” refers to inhibiting the progression of a cancer, an infectious disease, a neurodegenerative or amyloid disease, or an autoimmune disease, either physically, e.g., stabilization of a discernible symptom, physiologically, e.g., stabilization of a physical parameter, or both. In yet another embodiment, “treatment” or “treating” refers to delaying the onset of a cancer, a neurodegenerative or amyloid disease, or an autoimmune disease.

In certain embodiments, the compositions of the present invention are administered to a human subject as a preventative measure against such cancer, an infectious disease, a neurodegenerative or amyloid disease or autoimmune disease. As used herein, “prevention” or “preventing” refers to a reduction of the risk of acquiring a given cancer, infectious disease, neurodegenerative or amyloid disease, or autoimmune disease. In one mode of the embodiment, the compositions of the present invention are administered as a preventative measure to a human subject having a genetic predisposition to a cancer, infectious disease, neurodegenerative or amyloid disease or autoimmune disease. In another mode of the embodiment, the compositions of the present invention are administered as a preventative measure to a subject having a non-genetic predisposition to a cancer, or to a subject facing exposure to an agent of an infectious disease.

4.6.1. Target Infectious Diseases

Infectious diseases that can be treated or prevented by the methods of the present invention are caused by infectious agents including, but not limited to, viruses, bacteria, fungi protozoa and parasites.

Viral diseases that can be treated or prevented by the methods of the present invention include, but are not limited to, those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus, polio virus, human immunodeficiency virus type I (HIV-I), and human immunodeficiency virus type II (HIV-II).

Bacterial diseases that can be treated or prevented by the methods of the present invention are caused by bacteria including, but not limited to, mycobacteria, rickettsia, mycoplasma, neisseria and legionella.

Protozoal diseases that can be treated or prevented by the methods of the present invention are caused by protozoa including, but not limited to, leishmania, kokzidioa, and trypanosoma.

Parasitic diseases that can be treated or prevented by the methods of the present invention are caused by parasites including, but not limited to, chlamydia and rickettsia.

4.6.2. Target Autoimmune Diseases

Autoimmune diseases that can be treated by the methods of the present invention include, but are not limited to, insulin dependent diabetes mellitus (i.e., IDDM, or autoimmune diabetes), multiple sclerosis, systemic lupus erythematosus, Sjogren's syndrome, scleroderma, polymyositis, chronic active hepatitis, mixed connective tissue disease, primary biliary cirrhosis, pernicious anemia, autoimmune thyroiditis, idiopathic Addison's disease, vitiligo, gluten-sensitive enteropathy, Graves' disease, myasthenia gravis, autoimmune neutropenia, idiopathic thrombocytopenia purpura, rheumatoid arthritis, cirrhosis, pemphigus vulgaris, autoimmune infertility, Goodpasture's disease, bullous pemphigoid, discoid lupus, ulcerative colitis, and dense deposit disease. The diseases set forth above, as referred to herein, include those exhibited by animal models for such diseases, such as, for example non-obese diabetic (NOD) mice for IDDM and experimental autoimmune encephalomyelitis (EAE) mice for multiple sclerosis.

The methods of the present invention can be used to treat such autoimmune diseases by reducing or eliminating the immune response to the patient's own (self) tissue, or, alternatively, by reducing or eliminating a pre-existing autoimmune response directed at tissues or organs transplanted to replace self tissues or organs damaged by the autoimmune response.

4.6.3. Target Cancers

Cancers that can be treated or prevented by the methods of the present invention include, but are not limited to, human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenström's macroglobulinemia, and heavy chain disease. Specific examples of such cancers are described in the sections below.

In a specific embodiment the cancer is metastatic. In another specific embodiment, the patient having a cancer is immunosuppressed by reason of having undergone anti-cancer therapy (e.g., chemotherapy radiation) prior to administration of the compositions of the invention alone or in combination with sensitized APCs.

4.6.3.1 Colorectal Cancer Metastatic to the Liver

It has been estimated that approximately 226,600 Americans will be diagnosed with cancers of the digestive tract in 2000. Most notably, the colon will be the primary site for approximately 93,800 of these cases and the rectum the primary site for another approximately 36,400 cases. Further, it is predicted that approximately 47,700 will die of colon cancer and another 8,600 will die of rectal cancer (Cancer Facts & Figures 2000, American Cancer Society (ACS), Atlanta, Ga., 2000). 80 percent of patients who die of colon or rectal cancer have metastatic disease involving the liver. Most metastatic tumors of the liver are from gastrointestinal primaries. Unfortunately, the natural history of metastatic liver lesions carries a grave prognosis and systemic chemotherapy regimens have been unable to induce significant response rates or alter length of survival (Drebin et al., in Current Therapy In Oncology, ed. J. E. Niederhuber, B. C. Decker, Mosby, 1993, p.426).

Colorectal cancer initially spreads to regional lymph nodes and then through the portal venous circulation to the liver, which represents the most common visceral site of metastasis. The symptoms that lead patients with colorectal cancer to seek medical care vary with the anatomical location of the lesion. For example, lesions in the ascending colon frequently ulcerate, which leads to chronic blood loss in the stool.

Radical resection offers the greatest potential for cure in patients with invasive colorectal cancer. Before surgery, the CEA titer is determined. Radiation therapy and chemotherapy are used in patients with advanced colorectal cancer. Results with chemotherapeutic agents (e.g., 5-fluorouracil) are mixed and fewer than 25 percent of patients experience a greater than 50 percent reduction in tumor mass (Richards et al., 1986, J. Clin. Oncol. 4:565).

Patients with widespread metastasises have limited survival and systemic chemotherapy has little impact in this group of patients. In addition, systemically administered chemotherapy is often limited by the severity of toxicities associated with the various agents, such as severe diarrhea, mucositis and/or myelosuppression. Other techniques, including hepatic radiation, systemic chemotherapy, hepatic arterial ligation, tumor embolization and immunotherapy have all been explored, but, for the most part, have proven ineffectual in prolonging patient survival.

In a specific embodiment, the present invention provides compositions and methods for enhancing tumor specific immunity in individuals suffering from colorectal cancer metastasized to the liver, in order to inhibit the progression of the neoplastic disease. Preferred methods of treating these neoplastic diseases comprise administering a composition comprising an autologous preparation of hsp-antigenic molecule complexes and a saponin that is optionally covalently complexed to a tumor-antigenic moleculeic molecule, which elicits tumor-specific immunity against the tumor cells. Most specifically, the use of a composition of the invention in which the hsp is gp96, will result in nearly complete inhibition of liver cancer growth in cancer patients, without inducing toxicity and thus providing a dramatic therapeutic effect.

Accordingly, as an example of the method of the invention, gp96-antigenic molecule complexes are prepared from the cancer cells of a patient diagnosed with colorectal cancer, with or without liver metastases, combined with saponin fraction QS-21 and administered to the patient, via one of many different routes of administration, the preferred route being intradermally at different anatomical sites, e.g., left arm, right arm, left belly, right belly, left thigh, right thigh, etc. The site of injection will be varied for each weekly injection.

4.6.3.2 Hepatocellular Carcinoma

Hepatocellular carcinoma is generally a disease of the elderly in the United States. Although many factors may lead to hepatocellular carcinoma, the disease is usually limited to those persons with preexisting liver disease. Approximately 60 to 80 percent of patients in the United States with hepatocellular carcinoma have a cirrhotic liver and about four percent of individuals with a cirrhotic liver eventually develop hepatocellular carcinoma (Niederhuber (ed.), 1993, Current Therapy in Oncology, B. C. Decker, Mosby). The risk is highest in patients whose liver disease is caused by inherited hemochromatosis or hepatic B viral infection (Bradbear et al., 1985, J. Natl. Cancer Inst. 75:81; Beasley et al., 1981, Lancet 2:1129); hepatitis C virus infection has also emerged as a risk factor in the past decade (Colombo, 1999, Baillieres Best Pract Res Clin Gastroenterol 13(4):519-28). Other causes of cirrhosis that can lead to hepatocellular carcinoma include alcohol abuse and hepatic fibrosis caused by chronic administration of methotrexate. The most frequent symptoms of hepatocellular carcinoma are the development of a painful mass in the right upper quadrant or epigastrium, accompanied by weight loss. In patients with cirrhosis, the development of hepatocellular carcinoma is preceded by ascites, portal hypertension and relatively abrupt clinical deterioration. In most cases, abnormal values in standard liver function tests such as serum aminotransferase and alkaline phosphatase are observed.

CT scans of the liver are used to determine the anatomic distribution of hepatocellular carcinoma and also provide orientation for percutaneous needle biopsy. Approximately 70 percent of patients with hepatocellular carcinoma have an elevated serum alpha-fetoprotein concentration (McIntire et al., 1975, Cancer Res. 35:991) and its concentration correlates with the extent of the disease.

Radical resection offers the only hope for cure in patients with hepatocellular carcinoma. Such operative procedures are associated with five-year survival rates of 12 to 30 percent. Liver transplantation may improve survival of some younger individuals. However, most patients are not surgical candidates because of extensive cirrhosis multifocal tumor pattern or scarcity of compatible donor organs.

Chemotherapeutic agents have been administered either by intravenous route or through an intrahepatic arterial catheter. Such therapy has sometimes been combined with irradiation to the liver. Reductions in the size of measurable tumors of 50% or more have been reported in some patients treated with either systemic doxorubicin or 5-fluorouracil. However, chemotherapy often induces immunosuppression and rarely causes the tumor to disappear completely and the duration of response is short. The prognosis for patients with hepatocellular carcinoma is negatively correlated with cirrhosis and metastases to the lungs or bone. Median survival for patients is only four to six months. In a specific embodiment, the present invention provides compositions and methods for enhancing specific immunity in individuals suffering from hepatocellular carcinoma in order to inhibit the progression of the neoplastic disease and ultimately irradiate all preneoplastic and neoplastic cells. Thus the present invention also provides compositions comprising hsps or α2M, saponins, and hepatocellular carcinoma-antigenic molecules and methods for promoting immunity to the disease and for inhibiting cancer cell proliferation and metastasis. These compositions can be applied alone or in combination with each other or with biological response modifiers.

4.6.3.3 Breast Cancer

Another specific aspect of the invention relates to the treatment of breast cancer. The American Cancer Society estimated that in 2000, 184,200 American women will be diagnosed with breast cancer and 41,200 will succumb to the disease (Cancer Facts & Figures 2000, American Cancer Society (ACS), Atlanta, Georgia, 2000). This makes breast cancer the second major cause of cancer death in women, ranking just behind lung cancer. The treatment of breast cancer presently involves surgery, radiation, hormonal therapy and/or chemotherapy. Consideration of two breast cancer characteristics, hormone receptors and disease extent, has governed how hormonal therapies and standard-dose chemotherapy are sequenced to improve survival and maintain or improve quality of life. A wide range of multidrug regimens have been used as adjuvant therapy in breast cancer patients, including, but not limited to combinations of 2 cyclophosphamide, doxorubicin, vincristine methotrexate, 5-fluorouracil and/or leucovorin. In a specific embodiment, the present invention provides compositions comprising hsps or α2M, saponins, and breast cancer-antigenic molecules and methods for enhancing specific immunity to preneoplastic and neoplastic mammary cells in women. The present invention also provides compositions comprising hsps or α2M, saponins, and breast cancer-antigenic molecules and methods for preventing the development of neoplastic cells in women at enhanced risk for breast cancer, and for inhibiting cancer cell proliferation and metastasis. These compositions can be applied alone or in combination with each other or with biological response modifiers.

4.6.3.4 Autologous Antigenic Molecules

The specific immunogenicity of hsp or α2M-antigenic molecule complexes derives from the antigenic molecules therein. In a preferred embodiment of the invention, compositions of the invention comprising autologous antigenic molecules are employed in cancer immunotherapy. In one embodiment, the composition of the invention can comprise complexes of antigenic molecules and hsps purified from cancerous cells from the individual to whom they are to be administered. In other embodiments, the antigenic molecules are dissociated from the hsp-antigenic molecule complexes isolated from cancerous cells and incorporated into the compositions of the invention uncomplexed, covalently or non-covalently complexed to α2M, covalently complexed to a saponin such as QS-21, or covalently or non-covalently complexed to another hsp, for example a recombinant hsp. This approach offers the advantage of using antigenic molecules specific to cancers that are potentially antigenically distinct. Additionally, in autologous embodiments, cancer immunotherapy does not depend on the availability of cell lines or CTLs nor does it require definition of the antigenic epitopes of cancer cells.

4.6.4. Target Neurodegenerative or Amyloid Diseases

Neurodegenerative diseases that can be treated or prevent by the methods of the present invention include, but are not limited to, Alzheimer's Disease, age-related loss of cognitive function, senile dementia, Parkinson's disease, amyotrophic lateral sclerosis, Wilson's Disease, cerebral palsy, progressive supranuclear palsy, Guam disease, Lewy body dementia, prion diseases, spongiform encephalopathies, Creutzfeldt-Jakob disease, polyglutamine diseases, Huntington's disease, myotonic dystrophy, Freidrich's ataxia, ataxia, Gilles de la Tourette's syndrome, seizure disorders, epilepsy, chronic seizure disorder, stroke, brain trauma, spinal cord trauma, AIDS dementia, alcoholism, autism, retinal ischemia, glaucoma, autonomic function disorder, hypertension, neuropsychiatric disorder, schizophrenia, or schizoaffective disorder.

Amyloid diseases that can be treated or prevent by the methods of the present invention include but are not limited to are diseases characterized by the extracellular deposition of protein and/or peptide fibrils which form amyloid deposits or plaques, including but not limited to type II diabetes and amyloidoses associated with chronic inflammatory or infectious disease states and malignant neoplasms, e.g., myeloma. Certain amyloid disease such as Alzheimer's disease and prion diseases, e.g. Creutzfeldt Jacob disease, are neurodegenerative diseases.

4.7. Formation of Covalent Complexes

In certain embodiments of the present invention, antigenic molecules are covalently complexed to hsps, α2M and/or saponin. The covalent complexing of antigenic molecules to hsps, α2M and/or saponin is done prior to administration, before or after the compositions of the invention are formulated. Wherein covalent complexing of an endogenous hsp-peptide complex is desired, the complex is preferably cross-linked after purification from cells or tissues. In one embodiment, antigenic molecules are covalently coupled to hsps, α2M and/or saponin by chemical crosslinking. Chemical crosslinking methods are well known in the art. For example, in a preferred embodiment, glutaraldehyde crosslinking may be used. Glutaradehyde crosslinking has been used for formation of covalent complexes of peptides and hsps (see Barrios et al., 1992, Eur. J. Immunol. 22: 1365-1372). Preferably, 1-2 mg of complex is crosslinked in the presence of 0.002% glutaraldehyde for 2 hours. Glutaraldehyde is removed by dialysis against phosphate buffered saline (PBS) overnight (Lussow et al., 1991, Eur. J. Immunol. 21: 2297-2302).

In another embodiment, the antigenic molecules and hsps, α2M and/or saponin are crosslinked by ultraviolet (UV) crosslinking.

For α2M-antigenic molecule complexes, covalent complexes can be made by preparing the complexes according to the method described by Grøn and Pizzo, 1998, Biochemistry, 37: 6009-6014.

4.8. Combination with Adoptive Immunotherapy

Adoptive immunotherapy refers to a therapeutic approach for treating cancer or infectious diseases in which immune cells are administered to a host with the aim that the cells mediate either directly or indirectly specific immunity to tumor cells and/or antigenic components thereof or regression of the tumor or treatment of infectious diseases, as the case may be (see U.S. application Ser. No. 08/527,546, filed Sep. 13, 1995, now U.S. Pat. No. 5,985,270 issued Nov. 16, 1999, which is incorporated by reference herein in its entirety). As an optional step, in accordance with the methods described herein, APC are sensitized with hsps or α2M complexed with antigenic (or immunogenic) molecules and used in adoptive immunotherapy. See Binder et al., 2000, Nature Immunology 1(2), 151-154.

In a specific embodiment, therapy by administration of the claimed compositions comprising an hsp or α2M, an antigenic molecule, and a saponin, using any desired route of administration, may optionally be combined with adoptive immunotherapy using APC sensitized with hsp- or α2M-antigenic molecule complexes. As described in Section 4.10 herein, the hsp- or α2M-peptide complex-sensitized APC can be administered alone, in combination claimed compositions, or before or after administration of the claimed compositions. Furthermore, the mode of administration can be varied, including but not limited to, e.g., mucosally or intramuscularly, although intravenously, intradermally or subcutaneously is preferred.

4.8.1. Obtaining Antigen-Presenting Cells

The APCs, including but not limited to macrophages, dendritic cells and B-cells, are preferably obtained by production in vitro from stem and progenitor cells from human peripheral blood or bone marrow as described by Inaba et al., 1992, J. Exp. Med. 176:1693-1702.

APCs can be obtained by any of various methods known in the art. In a preferred aspect human macrophages are used, obtained from human blood cells. By way of example but not limitation, macrophages and dendritic cells can be obtained as follows:

Mononuclear cells are isolated from peripheral blood of a patient (preferably the patient to be treated), by Ficoll-Hypaque gradient centrifugation and are seeded on tissue culture dishes which are pre-coated with the patient's own serum or with other AB+human serum. The cells are incubated at 37° C. for 1 hour, then non-adherent cells are removed by pipetting. To the adherent cells left in the dish, is added cold (4° C.) 1 mM EDTA in phosphate-buffered saline and the dishes are left at room temperature for 15 minutes. The cells are harvested, washed with RPMI buffer and suspended in RPMI buffer. Increased numbers of macrophages may be obtained by incubating at 37° C. with macrophage-colony stimulating factor (M-CSF); increased numbers of dendritic cells may be obtained by incubating with granulocyte-macrophage-colony stimulating factor (GM-CSF) as described in detail by Inaba et al., 1992, J. Exp. Med. 176:1693-1702.

4.8.2. Sensitization of Antigen Presenting Cells with hsp- or α2M-Antigenic Molecule Complexes

APCs are sensitized with complexes of hsp or α2M and antigenic molecules, preferably by incubating the cells in vitro with the complexes. Sensitization is preferably achieved by incubating the APCs in vitro with the hsp- or α2M-antigenic molecule complexes at 37° C. for 15 minutes to 24 hours. By way of example but not limitation, 4×10⁷ macrophages can be incubated with 10 microgram gp96-peptide complexes per ml or 100 microgram hsp90-peptide complexes per ml at 37° C. for 15 minutes-24 hours in 1 ml plain RPMI medium. The cells are washed three times and resuspended in a preferably sterile physiological medium, at a convenient concentration (e.g., 1×10⁷/ml) for injection in a patient. Preferably, the patient into which the sensitized APCs are injected is the patient from which the APCs were originally isolated (autologous embodiment).

Optionally, the ability of sensitized APCs to stimulate, for example, the antigen-specific, class I-restricted cytotoxic T-lymphocytes (CTL) can be monitored by their ability to stimulate CTLs to release tumor necrosis factor, and by their ability to act as targets of such CTLs.

4.8.3. Reinfusion of Sensitized APCs

In one embodiment, APCs sensitized with complexes of hsp-antigenic molecule complexes or α2M-antigenic molecule complexes are reinfused into the patient systemically, preferably intravenously, by conventional clinical procedures. Patients generally receive from about 10⁶ to about 10¹² sensitized APCs, depending on the condition of the patient. In some regimens, patients may optionally receive in addition a suitable dosage of a biological response modifier including but not limited to the cytokines IFN-α, IFN-γ, IL-2, IL-4, IL-6, TNF or other cytokine growth factor.

4.9. Passive Immunotherapy

The hsp or α2M/antigenic molecule/saponin compositions of the invention can also be used for passive immunotherapy against cancers and infectious diseases. Passive immunity is the short-term protection of a host, achieved by the administration of pre-formed antibody directed against a heterologous organism. For example, compositions of the invention comprising antigenic molecules that display the antigenicity of an antigen of an infectious organism may be used to elicit an immune response in a subject, the sera removed from the subject and used for treatment or prevention of a disease caused the infectious organism in another subject.

4.10. Formulation, Administration and Kits

The hsp or α2M/antigenic molecule/saponin compositions of the present invention may be formulated into pharmaceutical preparations for administration to mammals, preferably humans, for treatment or prevention of cancer, autoimmune diseases or infectious diseases. Compositions comprising a compound of the invention formulated in a compatible pharmaceutical carrier may be prepared, packaged, and labeled for treatment of the indicated tumor(s), infectious disease or autoimmune disease.

Drug solubility and the site of absorption are factors which should be considered when choosing the route of administration of a therapeutic agent. In an embodiment of the invention, the present compositions may be administered using any desired route of administration, and preferably intradermally or subcutaneously. Advantages of intradermal or subcutaneous administration include use of lower doses and rapid absorption, respectively. As an alternative to intradermal or subcutaneous administration, the compositions of the invention can be administered mucosally. Mucosal routes of administration include, but are not limited to, oral, rectal and nasal administration. Preparations for mucosal administrations are suitable in various formulations as described below.

Water-soluble compositions of the invention may be formulated in an appropriate buffer, for example, phosphate buffered saline or other physiologically compatible solutions, preferably sterile. Alternatively, if the complex has poor solubility in aqueous solvents, then it may be formulated with a non-ionic surfactant such as Tween, or polyethylene glycol. The compositions of the invention may be formulated from a saponin powder or from a saponin stock aqueous solution by simple admixture with the hsp or α2M and antigenic molecules. The hsp, α2M or antigenic molecule may also be in lyophilized form, particularly when recombinant forms of these molecules are employed. Thus, the compounds and their physiologically acceptable solvates may be formulated for administration by inhalation or insufflation (either through the mouth or the nose) or oral, buccal, parenteral, or rectal administration or, in the case of tumors, directly injected into a solid tumor.

For oral administration, the pharmaceutical preparation may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art.

Preparations for oral administration may be suitably formulated to give controlled release of the active compound.

For buccal administration, the compositions may take the form of tablets or lozenges formulated in conventional manner.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example, subcutaneously or intramuscularly) or by intramuscular injection. Thus, for example, the compositions may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

The invention also provides kits for carrying out the therapeutic regimens of the invention. Such kits comprise in one or more containers therapeutically or prophylactically effective amounts of the hsp or α2M, uncomplexed or covalently or noncovalently complexed to an antigenic molecule, and a saponin. Kits directed to the treatment or prevention of cancer or infectious disease comprise an antigenic molecule, uncomplexed or complexed to hsp or α2M; and a saponin, uncomplexed or complexed to an antigenic molecule. Each kit component is preferably purified and in pharmaceutically acceptable form. Kits directed to the treatment or prevention of cancer or infectious disease optionally further comprise in a separate container, APCs, which may be sensitized. If the APCs are not sensitized, the kit may further comprise a purified antigenic molecule for sensitizing the APCs. The APCs are preferably purified. Each kit component may be lyophilized or dessicated (i.e., in powder form), or may be in the form of a pharmaceutically acceptable solution, e.g., in combination with sterile saline, dextrose solution, or buffered solution, or other pharmaceutically acceptable sterile fluid. Wherein one or more of the kit components is lyophilized or dessicated, the kit optionally further comprises in a container a pharmaceutically acceptable solution (e.g., saline, dextrose solution, etc.), preferably sterile, to reconstitute the material to form a solution for injection purposes.

In another embodiment, a kit of the invention further comprises a needle or syringe, preferably packaged in sterile form, for injecting the composition, and/or a packaged alcohol pad. Instructions are optionally included for administration of the compositions of the invention by a clinician or by the patient.

4.11. Monitoring of Effects During Cancer Prevention and Immunotherapy with the Compositions of the Invention

The effect of immunotherapy with the hsp or α2M/antigenic molecule/saponin compositions of the present invention on development and progression of neoplastic diseases can be monitored by any methods known to one skilled in the art, including but not limited to measuring: a) delayed hypersensitivity as an assessment of cellular immunity; b) activity of cytolytic T-lymphocytes in vitro; c) levels of tumor antigenic molecules, e.g., carcinoembryonic (CEA) antigens; d) changes in the morphology of tumors using techniques such as a computed tomographic (CT) scan; and e) changes in levels of putative biomarkers of risk for a particular cancer in individuals at high risk, and f) changes in the morphology of tumors using a sonogram.

4.11.1. Delaved Hypersensitivity Skin Test

Delayed hypersensitivity skin tests are of great value in the overall immunocompetence and cellular immunity to an antigen. Inability to react to a battery of common skin antigens is termed anergy (Sato et al., 1995, Clin. Immunol. Pathol. 74:35-43).

Proper technique of skin testing requires that the antigens be stored sterile at 4° C., protected from light and reconstituted shorted before use. A 25- or 27-gauge needle ensures intradermal, rather than subcutaneous, administration of antigen. Twenty-four and 48 hours after intradermal administration of the antigen, the largest dimensions of both erythema and induration are measured with a ruler. Hypoactivity to any given antigen or group of antigens is confirmed by testing with higher concentrations of antigen or, in ambiguous circumstances, by a repeat test with an intermediate test.

4.11.2. Activity of Cytolytic T-lymphocytes in vitro

8×10⁶ peripheral blood derived T lymphocytes isolated by the Ficoll-Hypaque centrifugation gradient technique, are restimulated with 4×10⁴ mitomycin C treated tumor cells in 3 ml RPMI medium containing 10% fetal calf serum. In some experiments, 33% secondary mixed lymphocyte culture supernatant or IL-2, is included in the culture medium as a source of T cell growth factors.

In order to measure the primary response of cytolytic T-lymphocytes after immunization, T cells are cultured without the stimulator tumor cells. In other experiments, T cells are restimulated with antigenically distinct cells. After six days, the cultures are tested for cytotoxicity in a 4 hour ⁵¹Cr-release assay. The spontaneous ⁵¹Cr-release of the targets should reach a level less than 20%. For the anti-MHC class I blocking activity, a tenfold concentrated supematant of W6/32 hybridoma is added to the test at a final concentration of 12.5% (Heike et al., J. Immunotherapy 15:165-174).

4.11.3. Levels of Tumor Antigenic Molecules

Although it may not be possible to detect unique tumor antigens on all tumors, many tumors display antigens that distinguish them from normal cells. The monoclonal antibody reagents have permitted the isolation and biochemical characterization of the antigens and have been invaluable diagnostically for distinction of transformed from nontransformed cells and for definition of the cell lineage of transformed cells. The best-characterized human tumor-associated antigens are the oncofetal antigens. These antigens are expressed during embryogenesis, but are absent or very difficult to detect in normal adult tissue. The prototype antigen is carcinoembryonic antigen (CEA), a glycoprotein found on fetal gut and human colon cancer cells, but not on normal adult colon cells. Since CEA is shed from colon carcinoma cells and found in the serum, it was originally thought that the presence of this antigen in the serum could be used to screen patients for colon cancer. However, patients with other tumors, such as pancreatic and breast cancer, also have elevated serum levels of CEA. Therefore, monitoring the fall and rise of CEA levels in cancer patients undergoing therapy has proven useful for predicting tumor progression and responses to treatment.

Several other oncofetal antigens have been useful for diagnosing and monitoring human tumors, e.g., alpha-fetoprotein, an alpha-globulin normally secreted by fetal liver and yolk sac cells, is found in the serum of patients with liver and germinal cell tumors and can be used as a marker of disease status.

4.11.4. Computed Tomographic (CT) Scan

CT remains the choice of techniques for the accurate staging of cancers. CT has proved more sensitive and specific than any other imaging techniques for the detection of metastases.

4.11.5. Measurement of Putative Biomarkers

The levels of a putative biomarker for risk of a specific cancer are measured to monitor the effect of hsp bound to peptide complexes. For example, in individuals at enhanced risk for prostate cancer, serum prostate-specific antigen molecule (PSA) is measured by the procedure described by Brawer et al., 1992, J. Urol. 147:841-845, and Catalona et al., 1993, JAMA 270:948-958; or in individuals at risk for colorectal cancer, CEA is measured by techniques well known in the art; and in individuals at enhanced risk for breast cancer, 16-α-hydroxylation of estradiol is measured by the procedure described by Schneider et al., 1982, Proc. Natl. Acad. Sci. USA 79:3047-3051.

4.11.6. Sonogram

A sonogram remains an alternative choice of technique for the accurate staging of cancers.

4.12. Combination Therapy for Cancer Treatment

The present hsp or α2M/antigenic molecule/saponin compositions can be administered together with treatment with irradiation or one or more chemotherapeutic agents. For irridiation treatment, the irradiation can be gamma rays or X-rays. For a general overview of radiation therapy, see Hellman, Chapter 12: Principles of Radiation Therapy Cancer, in: Principles and Practice of Oncology, DeVita et al., eds., 2nd. Ed., J. B. Lippencott Company, Philadelphia. Useful chemotherapeutic agents include methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel. In a specific embodiment, a composition of the invention is administered concurrently with radiation therapy or one or more chemotherapeutic agents. In another specific embodiment, chemotherapy or radiation therapy is administered prior or subsequent to administration of a present composition, preferably at least an hour, five hours, 12 hours, a day, a week, a month, more preferably several months (e.g., up to three months), subsequent to administration of a composition of the invention.

4.13. Dosages

In the dosage regimens recited below, an hsp or α2M composition, with or without complexed covalently or non-covalently bound antigenic molecules, is administered together with a saponin. Preferred routes of administration are either intradermal or subcutaneous, wherein the sites of administration are varied sequentially. For example, the dosage regimens may be given once weekly for a period of about 4 to 6 weeks, and the mode of administration may be varied with each administration. Further, each site of administration may be varied sequentially. Thus, by way of example and not limitation, the first injection can be given, either intradermally or subcutaneously, on the left arm, the second on the right arm, the third on the left belly, the fourth on the right belly, the fifth on the left thigh, the sixth on the right thigh, etc. The same site can be repeated after a gap of one or more injections. Also, split injections can be given. Thus, for example, half the dose can be given in one site and the other half in another site on the same day.

After 4-6 weeks, further injections are preferably given at two-week intervals over a period of time of one month. Later injections can be given monthly. The frequency of later injections can be modified depending upon the patient's clinical progress and responsiveness to the immunotherapy. Alternatively, the mode of administration is sequentially varied, e.g., weekly administrations are given in sequence intradermally or subcutaneously.

hsps or α2M and hsp or α2M -antigenic molecule complexes together with a saponin may be administered to mammalian subjects, e.g., primates, dogs, cats, mice, rats, horses, cows, pigs, etc., and preferably humans.

An amount of saponin from about 0.1 μg to about 1000 μg is preferred in a hsp or α2M/antigenic molecule/saponin composition of the invention. The saponin component of a composition of the invention can be anywhere within this range; e.g., 1, 2, 3, 5, 10, 15, 20, 25, 30, 40, 50, 60, 70, 80, 90, 100, 110, 125, 150, 200, 250, 300 or 500 μg saponin may be used. The most preferred saponin dose is generally about 100 μg.

It will be understood that, where reference is made to an antigenic molecule as a component of a composition, an antigenic peptide or full-length protein may be used (e.g. having more than 50 amino acid residues). The amount of an antigenic molecule, when administered as a complex, is determined by the amount which is bound covalently or non-covalently to an hsp or α2M. The amount of an antigenic molecule, when administered as free antigenic molecule together in a composition with an hsp or α2M and a saponin, is administered in the range of from 10 to 1500 μg, with from 25 to 100 μg being preferred; e.g., 10, 30, 50, 75, 100, 300, 500, 1000 or 1500 μg may be used.

In certain embodiments, a dose of the composition of the invention may comprise an amount of hsp or α2M/antigenic molecule/saponin that in the absence of one or more other components of the composition is not therapeutic amount, but is therapeutic when the components are combined. Accordingly, an immunogenic composition of the invention comprises can sub-immunogenic amounts of its individual components, i.e., comprises, for example, an hsp-antigenic molecule in an amount that is in itself non-immunogenic or sub-therapeutic, but is immunogenic or therapeutic when combined with a saponin according to the disclosed methods. Methods of assaying the immunogenicity of the individual components or subcombinations of the compositions of the invention are described in Section 4.4, supra.

4.13.1. Autoimmune Diseases

For the prevention or treatment of an autoimmune disease, the preferred dosages of the hsps or α2M in the compositions of the invention are generally about ten-fold lower than those disclosed in U.S. Pat. No. 6,007,821, which is incorporated herein by reference. For example, 0.1 μg to 1 mg of an hsp or α2M may be used, with 0.5-100 μg preferred and 1-10 μg most preferred. The hsp or α2M component of a composition can be anywhere within the 0.1 μg to 1 mg range; e.g., 0.2, 0.3, 0.5, 1, 3, 10, 30, 100, 300, 500 or 1000 μg may be used. Preferred routes of administration are intradermal and subcutaneous, with lower dosages preferred for the former and 5 to 10 fold higher dosages preferred for the latter.

Additional dosages of the hsps or α2M in the compositions of the invention range from about 0.05 μg to about 5000 μg, preferably from about 0.5 μg to about 1500 μg. In mammals, a range of from about 5 μg to about 500 μg, either intradermally or subcutaneously, is preferred, with from about 5 μg to about 200 μg subcutaneously and from about 0.5 μg to about 100 μg intradermally being most preferred.

4.13.2. Cancer and Infectious Diseases

For the prevention or treatment of cancer or infectious diseases, the preferred dosages of the the hsps or α2M components of the compositions of the invention are generally similar to those disclosed in U.S. Pat. No. 6,017,540, which is incorporated herein by reference. For example, 0.01 μg to 1 mg hsps or α2M may be used, with 1-10 μg preferred. The non-saponin component can be anywhere within the 0.01 μg to 1 mg range; e.g., 0.02, 0.05, 0.1, 0.2, 0.5, 1, 2, 5, 10, 20, 50, 100, 200, 500 or 1000 μg hsps or α2M may be used. Preferred routes of administration are intradermal and subcutaneous, with lower dosages preferred for the former and 5 to 10 fold higher dosages preferred for the latter.

4.13.3. Neurodegenerative and Amyloid Diseases

For the prevention or treatment of neurodegenerative and amyloid diseases, the preferred dosages of the hsps or α2M components of the compositions of the invention (optionally complexed to neurodegenerative disease- or amyloid disease-associated antigenic molecules) are generally similar to those disclosed in co-pending U.S. application Ser. Nos. 09/489,215 and 09/489,216, each of which is incorporated by reference herein in its entirety, although the dosages may be up to 10-fold less than the dosages disclosed in the foregoing U.S. applications. Generally, the dosage is in the range of about 0.5 μg to about 5000 μg for a human patient, the preferred human dosage being the same as used in a 25 g mouse. In specific embodiments, the non-saponin component can be anywhere within the 0.01 μg to 1 mg range; e.g., 0.02, 0.05, 0.1, 0.2, 0.5, 1, 2, 5, 10, 20, 50, 100, 200, 500 or 1000 μg hsps or α2M, preferably complexed to a neurodegenerative disease- or amyloid disease-associated antigenic molecule, may be used.

Compositions of the invention comprising the doses recited above are preferably given once weekly for a period of about 4-6 weeks, and the mode or site of administration is preferably varied with each administration.

In a specific embodiment, the compositions of the present invention for the prevention or treatment of a neurodegenerative disease are administered into the cerebrospinal fluid (CSF) of a patient by means of injection. In certain modes of the embodiment, administration is in or near the lesioned area of the nervous system.

In a preferred example, intradermal administrations are given, with each site of administration varied sequentially. Thus, by way of example and not limitation, the first injection may be given subcutaneously on the left arm, the second on the right arm, the third on the left belly, the fourth on the right belly, the fifth on the left thigh, the sixth on the right thigh, etc. The same site may be repeated after a gap of one or more injections. Also, split injections may be given. Thus, for example, half the dose may be given in one site and the other half on an other site on the same day.

Alternatively, the mode of administration is sequentially varied, e.g., weekly injections are given in sequence intradermally, intramuscularly, intravenously or intraperitoneally.

After 4-6 weeks, further injections are preferably given at two-week intervals over a period of time of one month. Later injections may be given monthly. The pace of later injections may be modified, depending upon the patient's clinical progress and responsiveness to the immunotherapy.

4.14. Methods of Making Pharmaceutical Compositions Comprising an hsp or α2M, an Antigenic Molecule and a Saponin

The present invention encompasses methods of making pharmaceutical compositions comprising an hsp or α2M, an antigenic molecule that displays the antigenicity of an antigen associated cancer, infectious disease, neurodegenerative or amyloid disease or autoimmune disease, and a saponin adjuvant, comprising combining the saponin, the antigenic molecule and the hsp or α2M under conditions that produce a pharmaceutical composition.

In certain embodiments, saponin is combined with the antigenic molecule and then the resulting mixture combined with the hsp or α2M. The saponin can be free of complexed molecules or complexed to another antigenic molecule. In one embodiment, the saponin and the antigenic molecule are subjected to conditions that promote formation of a covalent saponin-antigenic molecule complex prior to combining the mixture with the hsp or α2M.

In an embodiment where the saponin is combined with the antigenic molecule prior to combining the mixture with the hsp or α2M, the hsp or α2M is not complexed to any other molecule. In an alternative embodiment, the hsp or α2M is complexed to another antigenic molecule. The hsp or α2M can be covalently or non-covalently complexed to the second antigenic molecule. In yet another embodiment, the hsp or α2M is in the form of a fusion protein comprising the hsp or α2M and a second antigenic molecule.

Alternatively, the hsp or α2M can be combined with the antigenic molecule and then the resulting mixture can be combined with the saponin. In one embodiment, the antigenic molecule is combined with hsp or α2M under conditions that promote formation of a complex comprising the antigenic molecule and the hsp or α2M. Where the hsp or α2M and the antigenic molecule are combined prior to combining the mixture with the saponin, the saponin is optionally covalently attached to another antigenic molecule.

In an alternative embodiment, the hsp or α2M, the saponin, and the antigenic molecule are combined simultaneously. Optionally, the resulting mixture is subjected to conditions that promote covalent binding between the antigenic molecule and the hsp or α2M and/or between the antigenic molecule and the saponin.

For pharmaceutical compositions prepared by mixing an hsp- or α2M-antigenic molecule complex with a saponin, the hsp- or α2M-antigenic molecule complex can be prepared in vitro or purified from a cell or tissue, as described in Section 4.2, supra. The cell or tissue is optionally transfected with a nucleic acid encoding the hsp or α2M.

### 5. EXAMPLE

Administration of an hsp or α2M, Antigenic Molecule, and Saponin in Two UV-Induced Mouse Carcinoma Models

Two UV-induced carcinomas are available in C3H/HeN mice for testing the compositions and methods of the invention: (i) the highly-immunogenic 6138 carcinoma; and (ii) the less immunogenic 6139SJ carcinoma (Ward et al., 1989, J. Exp. Med. 170:217).

For example, an hsp such as gp96 is prepared from 6138 and 6139SJ carcinomas, or α2M is prepared from 6138 and 6139SJ carcinomas transfected with an α2M expression construct, by the procedures described in Section 4 above. The hsp or α2M, and the antigenic molecules derived therefrom, are then administered with saponin fraction QS-21 as an adjuvant.

5.1. Prevention Modality Using gp96

Materials and Methods. The ability of hsp or α2M, antigenic molecule, saponin compositions to prevent development of UV-induced carcinoma is tested as follows. Groups of female C₃H/HeN mice (obtained from the National Cancer Institute, Frederick, Md.), weighing approximately 25 g each, are used in the experiments set forth below.

Groups of mice are given twice at a ten day interval, either (i) phosphate buffer saline (PBS), (ii) 0.1, 1, 10, 25, 50, or 100 μg/mouse of, for example, gp96-peptide complexes derived from UV6138 carcinomas, or (iii) 0.1, 1, 10, 25, 50, or 100 μg/mouse of gp96-peptide complexes derived from UV3169SJ carcinoma. For each dosage of gp96-peptide complex, 0, 1.6, 10, 20, 50, or 100 μg of saponin fraction QS-21 (reconstituted in PBS from lyophilized powder) is mixed with the gp96-peptide complexes and coadministered. Control sets of mice receive the dosage series of QS-21 alone.

Mice are challenged with 10⁷ cells from either the UV6138 carcinoma or the UV6139SJ carcinoma 15 days after the second injection. Tumors are measured at two day intervals. Since the UV6138 tumor is a regressor tumor, mice are irradiated at 400 rad 10 days after the second injection with PBS or gp96 in order to permit growth of the tumor. The UV6139SJ challenged mice are not irradiated.

Results. It has previously been shown that administration of gp96 isolated from the UV6138 carcinoma rendered the mice immune to the UV6138 challenge but not the UV6139SJ challenge (see U.S. Pat. No. 5,837,251). Conversely, administration of gp96 isolated from the UV6139SJ conferred resistance to the UV6139SJ cells but not to the UV6138 cells. The resistance conferred by the gp96 derived from the UV6138 against the UV6138 cells was much greater (6 out of 7 mice) than the resistance conferred by the gp96 derived from the UV6139 against the UV6139SJ cells (2 out of 4 mice) (U.S. Pat. No. 5,837,251). These results indicate that administration of gp96 preparations derived from the two UV-induced carcinomas immunized syngeneic mice from the respective cancer cell type and that the resistance rendered was greater and more uniform against the more immunogenic carcinoma cells.

Coadministration of one or more saponins along with gp96-peptide complexes will elicit the desired immune response using reduced levels of the gp96-peptide complexes as compared to gp96-peptide complexes administered alone (i.e. in the absence of saponin). Accordingly, the invention provides the advantage of permitting reduction of the amount of an hsp or α2M-peptide complex required to elicit a desired immune response for prevention or treatment of cancer or infectious disease.

5.2. Treatment Modality Using gp96

Materials and Methods. The ability of gp96/antigenic molecule/saponin compositions to mediate therapy of pre-existing cancers is tested as follows. Six groups of mice are injected intradermally with 10⁷ cells of the UV6139SJ carcinoma. The mice are kept under observation until the tumors become visible and palpable at day four. Thereafter, mice in a first group receive no treatment. Mice in a second group receive, every other day for a total of five injections, 6, 1, 0.6, or 0.1 μg/mouse of gp96-peptide complex derived from UV6139SJ carcinoma cells. Mice in a third group receive, in a similar manner, a total of five injections of gp96-peptide complex derived from normal liver. Mice in a fourth group receive, in a similar manner, a total of five injections of gp96-peptide complex derived from the UV6139SJ carcinoma cells, mixed with 20 or 100 μg of QS-21. The mice in the fifth group receive, in a similar manner, a total of five injections of gp96-peptide complex derived from normal liver mixed with 20 or 100 μg QS-21. Finally, the mice in the sixth group receive 20 or 100 μg QS-21 alone.

Results. In prior experiments, tumor growth, monitored as the average of two perpendicular diameter measurements, was significantly retarded in mice treated with tumor-derived gp96 but not in mice treated with the liver-derived gp96 or in untreated mice (see U.S. Pat. No. 5,837,251). These results indicated a therapeutic effect of gp96 peptide complexes in the UV6139SJ carcinoma model. All mice eventually succumbed to tumor growth. A scrutiny of the kinetics of tumor growth in treated and control mice shows that administration of tumor-derived gp96-peptide complex had an immediate inhibitory effect on tumor growth and that the effect appears to diminish after treatment with gp96-peptide complex is terminated.

Coadministration of QS-21 with gp96-peptide complex derived from UV6139SJ carcinoma cells will result in a retardation of tumor growth when using smaller amounts of gp96-peptide complex as compared to the group receiving gp96-peptide complex from UV6139SJ carcinoma cells without QS-21. The administration of QS-21 alone, or gp96-peptide complex from normal liver plus QS-21, will not result in any significant difference as compared to the PBS control, or gp96-peptide complex from normal liver, respectively.

5.3. Analogous Experiments Using α2M or Other hsps

Use of α2M or other hsps in place of gp96 as described above will also result in both prevention of new tumors and retarded growth of established tumors using reduced amounts of the hsp or α2M when saponin adjuvant is present. Additionally, recombinantly-expressed antigenic peptide -α2M or -hsp fusion proteins can also be employed to elicit immunosuppression of new tumors and retarded growth of established tumors using reduced amounts of the hsp or α2M fusion proteins.

Accordingly, in analogous experiments, a saponin adjuvant such as QS-21 is added to the aforementioned preparations and administered. These α2M or hsp±QS-21 compositions are used in experiments that parallel those described above using gp96 compositions. Coadministration of QS-21 along with hsp- or α2M-complexes will result in a significant decrease in the amount of hsp- or α2M-complex required to elicit the desired immunosuppression of new tumors and retarded growth of established tumors.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Various publications are cited herein, the disclosures of each of which are incorporated by reference herein in their entireties.

What is claimed is:

1. A pharmaceutical composition comprising a purified heat shock protein (hsp) and a saponin.

2. The pharmaceutical composition of claim 1, further comprising a first antigenic molecule.

3. The pharmaceutical composition of claim 2, wherein the first antigenic molecule is bound to the hsp to form an hsp-antigenic molecule complex.

4. The pharmaceutical composition of claim 3, wherein the first antigenic molecule is non-covalently bound to the hsp.

5. A pharmaceutical composition comprising a purified alpha(2)macroglobulin (α2M) and a saponin.

6. The pharmaceutical composition of claim 5, further comprising a first antigenic molecule.

7. The pharmaceutical composition of claim 6, wherein the first antigenic molecule is bound to the α2M to form an α2M-antigenic molecule complex.

8. The pharmaceutical composition of claim 7, wherein the first antigenic molecule is non-covalently bound to the α2M.

9. The pharmaceutical composition of claim 2, wherein the first antigenic molecule is not bound to the saponin or to the hsp.

10. The pharmaceutical composition of claim 9, further comprising a second antigenic molecule.

11. The pharmaceutical composition of claim 10, wherein the second antigenic molecule is bound to the hsp.

12. The pharmaceutical composition of claim 11, wherein the second antigenic molecule is covalently bound to the hsp to form an hsp-second antigenic molecule complex.

13. The pharmaceutical composition of claim 11, wherein the second antigenic molecule is non-covalently bound to the hsp.

14. The pharmaceutical composition of claim 10, further comprising a third antigenic molecule.

15. The pharmaceutical composition of claim 14, wherein the third antigenic molecule is covalently bound to the saponin to form a saponin-third antigenic molecule complex.

16. The pharmaceutical composition of claim 10, wherein the second antigenic molecule is covalently bound to the saponin.

17. The pharmaceutical composition of claim 2, wherein the first antigenic molecule is bound to the hsp to form an hsp-first antigenic molecule complex.

18. The pharmaceutical composition of claim 17, wherein the first antigenic molecule is covalently bound to the hsp.

19. The pharmaceutical composition of claim 17, wherein the first antigenic molecule is non-covalently bound to the hsp.

20. The pharmaceutical composition of claim 17, further comprising a second antigenic molecule covalently bound to the saponin to form a saponin-second antigenic molecule complex.

21. The pharmaceutical composition of claim 2, wherein the first antigenic molecule is covalently bound to the saponin to form a saponin-first antigenic molecule complex.

22. The pharmaceutical composition of claim 2, wherein the hsp is hsp70, hsp90, gp96, calreticulin, hsp110, grp170, PDI, or a mixture of two or more of the foregoing.

23. The pharmaceutical composition of claim 2, wherein the amount of saponin is at least 1 microgram.

24. The pharmaceutical composition of claim 23, wherein the amount of saponin is 10 to 20 micrograms.

25. The pharmaceutical composition of claim 23, wherein the amount of saponin is 20 to 100 micrograms.

26. The pharmaceutical composition of claim 23, wherein the amount of saponin is 100 to 500 micrograms.

27. The pharmaceutical composition of claim 2, wherein the amount of hsp is at least 0.1 microgram.

28. The pharmaceutical composition of claim 27, wherein the amount of hsp is at least 1 microgram.

29. The pharmaceutical composition of claim 27, wherein the amount of hsp is no greater than 10 micrograms.

30. The pharmaceutical composition of claim 1 or 5, wherein the saponin is QS-7, QS-21, QS-21-V1, or QS-21-V2.

31. The pharmaceutical composition of claim 30, wherein the saponin is QS-21.

32. The pharmaceutical composition of claim 1, wherein the hsp is recombinant.

33. The pharmaceutical composition of claim 2 or 6, wherein the first antigenic molecule displays antigenicity of a tumor-associated antigen.

34. The pharmaceutical composition of claim 33, wherein the tumor is a fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, polycythemia vera, lymphoma, multiple myeloma, Waldenström's macroglobulinemia, or heavy chain disease

35. The pharmaceutical composition of claim 2 or 6, wherein the first antigenic molecule displays antigenicity of an antigen of an agent of infectious disease.

36. The pharmaceutical composition of claim 35, wherein the infectious disease is a viral disease.

37. The pharmaceutical composition of claim 36, wherein the virus is hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus, polio virus, human immunodeficiency virus type I (HIV-I), and human immunodeficiency virus type II (HIV-II).

38. The pharmaceutical composition of claim 35, wherein the infectious disease is a bacterial disease.

39. The pharmaceutical composition of claim 38, wherein the bacterial is mycobacteria rickettsia, mycoplasma, neisseria and legionella.

40. The pharmaceutical composition of claim 35, wherein the infectious disease is a protozoal disease.

41. The pharmaceutical composition of claim 40, wherein the protozoa is leishmania, kokzidioa, or trypanosoma.

42. The pharmaceutical composition of claim 2 or 6, wherein the first antigenic molecule displays antigenicity of an antigen associated with a neurodegenerative disorder.

43. The pharmaceutical composition of claim 42, wherein the first antigenic molecule is β-amyloid or a fragment thereof, an oligomeric Aβ complex or a fragment thereof, an ApoE4-Aβ complex or a fragment thereof, tau protein or a fragment thereof, a mutant amyloid precurser protein or a fragment thereof, a mutant of presenillin or a fragment thereof, α-synuclein or a fragment thereof, or a prion protein or a fragment thereof.

44. The pharmaceutical composition of claim 42, wherein the neurodegenerative disorder is Alzheimer's Disease, age-related loss of cognitive function, senile dementia, Parkinson's disease, amyotrophic lateral sclerosis, Wilson's Disease, cerebral palsy, progressive supranuclear palsy, Guam disease, Lewy body dementia, a prion disease, a spongiform encephalopathy, Creutzfeldt-Jakob disease, a polyglutamine disease, Huntington's disease, myotonic dystrophy, Freidrich's ataxia, ataxia, Gilles de la Tourette's syndrome, seizure disorders, epilepsy, chronic seizure disorder, stroke, brain trauma, spinal cord trauma, AIDS dementia, alcoholism, autism, retinal ischemia, glaucoma, autonomic function disorder, hypertension, neuropsychiatric disorder, schizophrenia, or schizoaffective disorder.

45. The pharmaceutical composition of claim 11, wherein the hsp-second antigenic molecule complex is purified from a cancerous cell.

46. The pharmaceutical composition of claim 11, wherein the hsp-second antigenic molecule complex is purified from a cell infected with an agent of infectious disease.

47. The pharmaceutical composition of claim 17, wherein the hsp-first antigenic molecule complex is purified from a cancerous cell.

48. The pharmaceutical composition of claim 17, wherein the hsp-first antigenic molecule complex is purified from a cell infected with an agent of infectious disease.

49. The pharmaceutical composition of claim 11, wherein the hsp-second antigenic molecule complex is prepared in vitro.

50. The pharmaceutical composition of claim 17, wherein the hsp-first antigenic molecule complex is prepared in vitro.

51. The pharmaceutical composition of claim 2, wherein the hsp is part of a fusion protein comprising the first antigenic molecule.

52. The pharmaceutical composition of claim 10, wherein the hsp is part of a fusion protein comprising the second antigenic molecule.

53. The pharmaceutical composition of claim 52, wherein the first antigenic molecule is not bound to the saponin or to the α2M.

54. The pharmaceutical composition of claim 53, further comprising a second antigenic molecule.

55. The pharmaceutical composition of claim 54, wherein the second antigenic molecule is bound to the α2M.

56. The pharmaceutical composition of claim 55, wherein the second antigenic molecule is covalently bound to the α2M to form an α2M-second antigenic molecule complex.

57. The pharmaceutical composition of claim 55, wherein the second antigenic molecule is non-covalently bound to the α2M.

58. The pharmaceutical composition of claim 54, further comprising a third antigenic molecule.

59. The pharmaceutical composition of claim 58, wherein the third antigenic molecule is covalently bound to the saponin to form a saponin-third antigenic molecule complex.

60. The pharmaceutical composition of claim 54, wherein the second antigenic molecule is covalently bound to the saponin.

61. The pharmaceutical composition of claim 52, wherein the first antigenic molecule is bound to the α2M to form an α2M-first antigenic molecule complex.

62. The pharmaceutical composition of claim 61, wherein the first antigenic molecule is covalently bound to the α2M.

63. The pharmaceutical composition of claim 61, wherein the first antigenic molecule is non-covalently bound to the α2M.

64. The pharmaceutical composition of claim 61, further comprising a second antigenic molecule covalently bound to the saponin to form a saponin-second antigenic molecule complex.

65. The pharmaceutical composition of claim 52, wherein the first antigenic molecule is covalently bound to the saponin to form a saponin-first antigenic molecule complex.

66. The pharmaceutical composition of claim 52, wherein the amount of saponin is at least 1 microgram.

67. The pharmaceutical composition of claim 66, wherein the amount of saponin is 10 to 20 micrograms.

68. The pharmaceutical composition of claim 66, wherein the amount of saponin is 20 to 100 micrograms.

69. The pharmaceutical composition of claim 66, wherein the amount of saponin is 100 to 500 micrograms.

70. The pharmaceutical composition of claim 52, wherein the amount of hsp is at least 0.1 microgram.

71. The pharmaceutical composition of claim 70, wherein the amount of hsp is at least 1 microgram.

72. The pharmaceutical composition of claim 70, wherein the amount of hsp is no greater than 10 micrograms.

73. The pharmaceutical composition of claim 52, wherein the saponin is QS-21.

74. The pharmaceutical composition of claim 52, wherein the α2M is recombinant.

75. The pharmaceutical composition of claim 52, wherein the first antigenic molecule displays antigenicity of a tumor-associated antigen.

76. The pharmaceutical composition of claim 52, wherein the first antigenic molecule displays antigenicity of an antigen of an agent of infectious disease.

77. The pharmaceutical composition of claim 52, wherein the α2M is part of a fusion protein comprising the first antigenic molecule.

78. The pharmaceutical composition of claim 54, wherein the α2M is part of a fusion protein comprising the second antigenic molecule.

79. A method of eliciting an immune response against cancer or an agent of infectious disease in an individual comprising administering to the individual an amount of a pharmaceutical composition comprising a purified hsp, a first antigenic molecule that displays antigenicity of an antigen of said type of cancer or of an antigen of said agent of infectious disease, and a saponin, which amount is effective to elicit an immune response in the individual.

80. The method according to claim 79 in which the individual is a mammal.

81. The method according to claim 80 in which the mammal is a human.

82. The method according to claim 79, further comprising administering to the individual an effective amount of a biological response modifier selected from the group consisting of interferon-α, interferon-γ, interleukin-2, interleukin-4, interleukin-6, and tumor necrosis factor.

83. The method according to claim 79 in which the pharmaceutical composition is administered at weekly intervals.

84. The method according to claim 79 in which the pharmaceutical composition is administered intramuscularly, subcutaneously, intraperitoneally, intravenously, intradermally or mucosally.

85. A method of eliciting an immune response against cancer or an agent of infectious disease in an individual comprising administering to the individual an amount of a pharmaceutical composition comprising a purified α2M, a first antigenic molecule that displays antigenicity of an antigen of said type of cancer or of an antigen of said agent of infectious disease, and a saponin, which amount is effective to elicit an immune response in the individual.

86. A method of treating or preventing cancer in an individual in whom such treatment or prevention is desired, comprising administering to the individual an amount of a pharmaceutical composition comprising a purified hsp, a first antigenic molecule that displays antigenicity of an antigen of said type of cancer, and a saponin, which amount is effective to treat or prevent cancer in the individual.

87. A method of treating or preventing cancer in an individual in whom such treatment or prevention is desired, comprising administering to the individual an amount of a pharmaceutical composition comprising a purified α2M, a first antigenic molecule that displays antigenicity of an antigen of said type of cancer, and a saponin, which amount is effective to treat or prevent cancer in the individual.

88. A method of treating or preventing an infectious disease in an individual in whom such treatment or prevention is desired, comprising administering to the individual an amount of a pharmaceutical composition comprising a purified hsp, a first antigenic molecule that displays antigenicity of an agent of said infectious disease, and a saponin, which amount is effective to treat or prevent the infectious disease in the individual.

89. A method of treating or preventing a neurodegenerative or amyloid disease in an individual in whom such treatment or prevention is desired, comprising administering to the individual an amount of a pharmaceutical composition comprising a purified α2M, a first antigenic molecule that displays antigenicity of antigen associated with said neurodegenerative or amyloid disease, and a saponin, which amount is effective to treat or prevent cancer in the individual.

90. A method of treating or preventing a neurodegenerative or amyloid disease in an individual in whom such treatment or prevention is desired, comprising administering to the individual an amount of a pharmaceutical composition comprising a purified hsp, a first antigenic molecule that displays antigenicity of antigen associated with said neurodegenerative or amyloid disease, and a saponin, which amount is effective to treat or prevent cancer in the individual.

91. A method of treating or preventing an infectious disease in an individual in whom such treatment or prevention is desired, comprising administering to the individual an amount of a pharmaceutical composition comprising a purified α2M, a first antigenic molecule that displays antigenicity of an agent of said infectious disease, and a saponin, which amount is effective to treat or prevent the infectious disease in the individual.

92. A method of treating or preventing an autoimmune disease in an individual in whom such treatment or prevention is desired, comprising administering to the individual an amount of a pharmaceutical composition comprising a purified hsp and a saponin, which amount is effective to treat or prevent the autoimmune disease in the individual.

93. The method of claim 92, wherein the pharmaceutical composition further comprises an antigenic molecule.

94. A method of treating or preventing an autoimmune disease in an individual in whom such treatment or prevention is desired, comprising administering to the individual an amount of a pharmaceutical composition comprising a purified α2M and a saponin, which amount is effective to treat or prevent the autoimmune disease in the individual.

95. The method of claim 94, wherein the pharmaceutical composition further comprises an antigenic molecule.

96. A method of making a pharmaceutical composition comprising an hsp or α2M, a first antigenic molecule that displays the antigenicity of an antigen associated cancer, infectious disease, neurodegenerative disease or autoimmune disease, and a saponin adjuvant, said method comprising:
combining the saponin, the first antigenic molecule and the hsp or α2M under conditions that produce a pharmaceutical composition.

97. The method of claim 96, wherein the saponin is combined with the first antigenic molecule and then with the hsp or α2M.

98. The method of claim 97, wherein the saponin is combined with the first antigenic molecule under conditions that do not promote to covalent binding between the saponin and the first antigenic molecule.

99. The method of claim 97, wherein the saponin is combined with the first antigenic molecule under conditions that promote covalent binding between the saponin and the first antigenic molecule.

100. The method of claim 97, wherein the hsp or α2M is not complexed to any other molecule.

101. The method of claim 97, wherein the hsp or α2M is complexed to a second antigenic molecule.

102. The method of claim 101, wherein the hsp or α2M is covalently complexed to the second antigenic molecule.

103. The method of claim 101, wherein the hsp or α2M is non-covalently complexed to the second antigenic molecule.

104. The method of claim 97, wherein the hsp or α2M is in the form of a fusion protein comprising the hsp or α2M and a second antigenic molecule.

105. The method of claim 96, wherein the hsp or α2M is combined the first antigenic molecule and then with the saponin.

106. The method of claim 105, wherein the first antigenic molecule is combined with hsp or α2M under conditions that promote formation of a complex comprising the first antigenic molecule and the hsp or α2M.

107. The method of claim 105, wherein the saponin is covalently attached to a second antigenic molecule.

108. The method of claim 96, wherein the hsp or α2M, the saponin, and the first antigenic molecule are combined simultaneously.

109. The method of claim 108, further comprising subjecting the hsp or α2M, a saponin, and the first antigenic molecule to conditions that promote covalent binding between the first antigenic molecule and the hsp or α2M or between the first antigenic molecule and the saponin.

110. The method of claim 96, wherein the hsp or α2M is in the form of a complex with the first antigenic molecule.

111. The method of claim 110, wherein the complex is purified from a cell or tissue.

112. The method of claim 111, wherein the cell is a cancerous cell.

113. The method of claim 111, wherein the cell expresses a tumor antigen.

114. The method of claim 111, wherein the cell is infected with an infectious agent.

115. The method of claim 111, wherein the cell expresses an antigen of an infectious agent.

116. The method of claim 111, wherein the cells is transfected with a nucleic acid encoding the hsp or α2M.

117. The method of claim 96, wherein the hsp or α2M is covalently bound to the first antigenic molecule.

118. The method of claim 96, wherein the hsp or α2M is in the form of a fusion protein comprising the hsp or α2M and the first antigenic molecule.

119. The method of claim 96, wherein the saponin is covalently bound to the first antigenic molecule.
